(19) United States
(12) Patent Application Publication
(10) Pub. No.: US 2024/0390434 A1
(43) Pub. Date: Nov. 28, 2024

(54) PROBIOTIC ENHANCERS AND USES THEREOF

(71) Applicant: Blis Technologies Limited, Dunedin (NZ)

(72) Inventors: Nicola Jones, Dunedin (NZ); John David Hale, Dunedin (NZ); John Tagg, Dunedin (NZ); Rohit Jain, Dunedin (NZ); Liam Karl Harold, Dunedin (NZ)

(73) Assignee: Blis Technologies Limited, Dunedin (NZ)

(21) Appl. No.: 18/697,281

(22) PCT Filed: Sep. 30, 2022
(86) PCT No.: PCT/IB2022/059321

§ 371 Date: Mar. 29, 2024

(30) Foreign Application Priority Data
Sep. 30, 2021 (AU) 2021903132

(51) Int. Cl. A61K 35/744; A61K 8/60; A61K 8/99; A61K 35/00; A61K 47/26; A61P 31/04; A61Q 11/00

(52) U.S. Cl. CPC A61K 35/744; A61K 8/60; A61K 8/99; A61K 47/26; A61P 31/04; A61Q 11/00; A61K 2035/115

(57) ABSTRACT

Described herein is a method of improving the inhibitory profile of Streptococcus salivarius comprising formulating the S. salivarius in a composition comprising an effective amount of a supplemental saccharide. Also described herein are compositions comprising Streptococcus salivarius and supplemental saccharide, methods of inhibiting microorganisms using such compositions, therapeutic formulations comprising such compositions, and methods of treating or preventing diseases using such compositions and therapeutic formulations.

### FIELD OF THE INVENTION

The present invention broadly relates to methods for enhancing the effectiveness of Streptococcus salivarius probiotics, and to compositions useful in such methods.

### BACKGROUND TO THE INVENTION

Probiotics are live microorganisms that may provide various health benefits when consumed by, or administered to a subject. For example, Lactobacillus spp., and Bifidobacterium spp. are well known for use in maintaining or improving gut health.

A growing probiotics market has led to an increased focus on how to enhance the effectiveness of probiotic strains. To date, much of the focus has been on how prebiotics can alter the gut microbiome, or increase the effectiveness of probiotics useful for gut health. As a result of that work, various prebiotics which induce the growth or activity of gut probiotics have been identified. Such prebiotics include nondigestible fibre compounds, resistant starches, arabinogalactans, and oligosaccharides such as inulin and galacto-oligosaccharides.

Prebiotics were originally described as a “non-digestible food ingredient that beneficially affects the host by selectively stimulating the growth and/or activity of one or a limited number of bacteria in the colon, and thus improves human health”. By this definition, only a limited number of carbohydrates fit the criteria and they were considered for the promotion of bacteria residing in the colon. Since this description was conceived prebiotics have been defined as “a selectively fermented ingredient that results in specific changes in the composition and/or activity of the gastrointestinal microbiota, thus conferring benefit(s) upon host health”. The currently accepted definition excludes microbiota exclusively present in the oral cavity, however.

WO2016172658 describes use of a microbiome regulator such as a sugar or sugar alcohol for improving the growth and/or colonisation of beneficial bacteria in the large or small intestine. WO2004074496 describes the use of galacto-oligosaccharides for increasing beneficial bacteria in the gastrointestinal tract. US20200030366 describes methods for treating or preventing colonisation by gastrointestinal pathogenic microorganisms, comprising administering dietary fibre such as inulin. The compositions may also comprise prebiotics which include a range of sugars/saccharides such as sucrose, maltose, lactose, fructose, galactose, glucose, raffinose, mannose, ribose, and trehalose.

More recently, probiotics for oral application or orally targeted probiotics have been identified. S. salivarius probiotics are known for use against various oral pathogens. For example, S. salivarius strain K12 (“K12”) is documented for use in preventing or treating ear, nose and throat infections such as those caused by Streptococcus pyogenes (see for example WO2001027143 Blis Technologies Ltd). K12 is also known for use in the treatment of halitosis caused by anaerobic bacteria (see for example WO2005007178 Blis Technologies Ltd). S. salivarius strain Mia (herein “M18”) is known for use in the treatment of dental caries caused by Streptococcus mutans (see for example WO2003070919 Blis Technologies Ltd).

US20190336428 describes selectively increasing the growth of beneficial bacteria in the oral cavity using a saccharide selected from D-turanose, D-melezitose, D-lactitol, myo-inositol, and N-acetyl-D-mannosamine. WO2012065811 describes nutritional compositions for children that may comprise probiotics and prebiotics. US20160166501 describes oral compositions comprising Lactobacillus helveticus for use in oral hygiene. The compositions may also comprise other probiotic strain and excipients. WO2007144334 describes a composition for treating otitis media comprising a probiotic Lactobacillus strain and a bacterial strain capable of exerting bacteriostatic effects such as S. salivarius K12. The composition may be in the form of an infant formula comprising lactose.

US20190343899 describes a hard candy or toffee composition comprising prebiotics, prepared at high temperatures. WO2017129639 describes an infant formula comprising oligosaccharides.

Simple sugars are known to be used as prebiotics to promote the growth of bacteria by being consumed as an energy source. As bacteria utilise these sugars, they may produce organic acids as a by-product. These acids can have a weak non-selective inhibitory effect on the growth of other bacteria. Further, if used in the oral cavity, the production of acidic by-products can lower the environmental pH and cause the erosion of tooth enamel which can progress to tooth decay. A more acidic environment also promotes the growth of harmful dental pathogens such as S. mutans.

There is a need to identify alternatives to common sugars to promote antimicrobial activity in probiotics without significant influence on pH. There is also a need to identify alternatives to common sugars which enhance colonisation efficacy of probiotics to impart health benefits. There is also a need for compositions that can enhance the activity of probiotics in areas other than the gastrointestinal tract. It is an object of the present invention to go some way to meeting any one or more of these needs; and/or to at least provide the public with a useful choice.

Other objects of the invention may become apparent from the following description which is given by way of example only.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the invention provides a method of improving the inhibitory profile of Streptococcus salivarius comprising formulating the S. salivarius in a composition comprising an effective amount of a supplemental saccharide, wherein the Streptococcus salivarius is Streptococcus salivarius M18, Streptococcus salivarius K12, or a combination thereof, and wherein the supplemental saccharide is galactose, or raffinose, or a combination thereof.

In a second aspect, the invention provides a method for upregulating one or more genes in Streptococcus salivarius, comprising formulating the S. salivarius in a composition comprising an effective amount of a supplemental saccharide,
wherein the Streptococcus salivarius is Streptococcus salivarius M18, Streptococcus salivarius K12, or a combination thereof, and
wherein the supplemental saccharide is galactose, or raffinose, or a combination thereof.

In a third aspect, the invention provides a method of inhibiting a skin, dental, oral, mucosal and/or ENT microorganism, the method comprising contacting the microorganism with a composition comprising Streptococcus salivarius and an effective amount of a saccharide,
wherein the Streptococcus salivarius is Streptococcus salivarius M18, Streptococcus salivarius K12, or a combination thereof, and
wherein the supplemental saccharide is galactose or raffinose or a combination thereof.

In a fourth aspect, the invention provides a method for increasing production of one or more of a lantiobiotic peptide, bacteriocin or urease by Streptococcus salivarius, comprising formulating the S. salivarius in a composition comprising an effective amount of a supplemental saccharide,
wherein the Streptococcus salivarius is Streptococcus salivarius M18, Streptococcus salivarius K12, or a combination thereof, and
wherein the supplemental saccharide is galactose, or raffinose, or a combination thereof, and the production is increased relative to a composition lacking the supplemental saccharide.

In a fifth aspect, the invention provides a composition comprising Streptococcus salivarius and an effective amount of a supplemental saccharide for use in improving the inhibitory profile of the Streptococcus salivarius,
wherein the Streptococcus salivarius is Streptococcus salivarius M18, Streptococcus salivarius K12, or a combination thereof, and
wherein the supplemental saccharide is galactose or raffinose or a combination thereof.

In a sixth aspect, the invention provides a composition comprising Streptococcus salivarius K12, and raffinose in an amount of 2 to 3% by weight.

In a seventh aspect, the invention provides a composition comprising Streptococcus salivarius K12, and galactose in an amount of 0.25 to 0.75% by weight.

In an eighth aspect, the invention provides a composition comprising Streptococcus salivarius M18, and raffinose in an amount of 2 to 3% by weight.

In a ninth aspect, the invention provides a composition comprising Streptococcus salivarius M18, and galactose in an amount of 0.25 to 0.75% by weight.

In a tenth aspect, the invention provides a composition comprising Streptococcus salivarius K12, Streptococcus salivarius M18, and raffinose in an amount of 2 to 3% by weight.

In an eleventh aspect, the invention provides a composition comprising Streptococcus salivarius K12, Streptococcus salivarius M18, and galactose in an amount of 0.25 to 0.75% by weight.

In a twelfth aspect, the invention provides a composition comprising Streptococcus salivarius K12, Streptococcus salivarius M18, raffinose in an amount of 2 to 3% by weight, and galactose in an amount of 0.25 to 0.75% by weight.

In a thirteenth aspect, the invention provides a composition comprising Streptococcus salivarius K12, raffinose in an amount of 1.2 to 2.2% by weight, and galactose in an amount of 0.7 to 1.7% by weight.

In a fourteenth aspect, the invention provides a composition comprising Streptococcus salivarius M18, raffinose in an amount of 1.2 to 2.2% by weight, and galactose in an amount of 0.7 to 1.7% by weight.

In a fifteenth aspect, the invention provides a composition comprising Streptococcus salivarius K12, Streptococcus salivarius M18, raffinose in an amount of 1.2 to 2.2% by weight, and galactose in an amount of 0.7 to 1.7% by weight.

In a sixteenth aspect, the invention provides a therapeutic formulation comprising the composition of any one of the fifth to fifteenth aspects.

In a seventeenth aspect, the invention provides a method of treating or preventing a disease or disorder comprising administering to subject in need thereof a composition of any one of the fifth to fifteenth aspects, or a therapeutic formulation of the sixteenth aspect.

In an eighteenth aspect, the invention provides a method of inhibiting a microorganism sensitive to Blis-producing S. salivarius, the method comprising administering to subject in need thereof a composition of any one of the fo fifth to fifteenth aspects, or a therapeutic formulation of the sixteenth aspect.

In a nineteenth aspect, the invention relates to use of Streptococcus salivarius and a supplemental saccharide in the manufacture of a medicament for:
(a) the treatment or prevention of a disease or disorder, or
(b) the inhibition of a microorganism sensitive to Blis-producing S. salivarius,
   wherein the Streptococcus salivarius is Streptococcus salivarius M18, Streptococcus salivarius K12, or a combination thereof, and
   wherein the supplemental saccharide is galactose or raffinose or a combination thereof.

In a twentieth aspect, the invention provides a composition comprising Streptococcus salivarius and an effective amount of a supplemental saccharide for use in:
(a) the treatment or prevention of a disease or disorder, or
(b) the inhibition of a microorganism sensitive to Blis-producing S. salivarius,
   wherein the Streptococcus salivarius is Streptococcus salivarius M18, Streptococcus salivarius K12, or a combination thereof, and
   wherein the supplemental saccharide is galactose or raffinose or a combination thereof.

In a twenty-first aspect, the invention provides a method of manufacturing a composition comprising Streptococcus salivarius and an effective amount of a supplemental saccharide, the method comprising:
(a) combining Streptococcus salivarius with supplemental saccharide, and
(b) mixing to produce a homogeneous blend:
   wherein the Streptococcus salivarius is Streptococcus salivarius M18, Streptococcus salivarius K12, or a combination thereof, and
   wherein the supplemental saccharide is galactose or raffinose or a combination thereof.

In a twenty-second aspect, the invention relates to use of a composition manufactured by the method of the twenty-first aspect for the treatment or prevention of a disease or disorder, or for the inhibition of a microorganism sensitive to Blis-producing S. salivarius.

The following embodiments and preferences may relate alone or in any combination of any two or more to any of the above aspects.

In various embodiments, the upregulated gene(s) encodes for a lantibiotic peptide or bacteriocin.

In various embodiments, the upregulated gene(s) encodes for a Class I lantibiotic peptide or Class II bacteriocin.

In various embodiments, the lantibiotic peptide is salA, salB, sal9 or a combination thereof.

In various embodiments, the lantibiotic peptide is salA, salB, or a combination thereof.

In various embodiments, the bacteriocin is salQ.

In various embodiments, the upregulated gene(s) encodes for a subunit of a urease protein.

In various embodiments, the urease is ureC.

In various embodiments, at least one of the upregulated gene(s) comprises or consists of a polynucleotide sequence with at least 70% sequence identity to any one of SEQ ID NOs 15-22, or at least one of the upregulated gene(s) comprises or consists of a polynucleotide sequence that encodes a polypeptide with at least 70% sequence identity to any one of SEQ ID NOs 23-30.

In various embodiments, at least one of the upregulated gene(s) comprises or consists of a polynucleotide sequence with at least 70% identity to any one of SEQ ID NOs 15-22, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOs 15-22.

In various embodiments, at least one of the upregulated gene(s) comprises or consists of a polynucleotide sequence that encodes a polypeptide with at least 70% identity to any one of SEQ ID NOs 23-30, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOs 23-30.

In various embodiments:
a. at least one of the upregulated gene(s) is a salA gene or variant thereof comprising or consisting of a polynucleotide sequence with at least 70% identity to SEQ ID NO 15 or 19, or encoding a polypeptide with at least 70% identity to SEQ ID NO 23 or 27;
b. at least one of the upregulated gene(s) is a salB gene or variant thereof comprising or consisting of a polynucleotide sequence with at least 70% identity to SEQ ID NO 16, or encoding a polypeptide with at least 70% identity to SEQ ID NO 24;
c. at least one of the upregulated gene(s) is a salQ gene or variant thereof comprising or consisting of a polynucleotide sequence with at least 70% identity to SEQ ID NO 17 or 21, or encoding a polypeptide with at least 70% identity to SEQ ID NO 25 or 29;
d. at least one of the upregulated gene(s) is a sal9 gene or variant thereof comprising or consisting of a polynucleotide sequence with at least 70% identity to SEQ ID NO 20, or encoding a polypeptide with at least 70% identity to SEQ ID NO 28; and/or
e. at least one of the upregulated gene(s) is a ureC gene or variant thereof comprising or consisting of a polynucleotide sequence with at least 70% identity to SEQ ID NO 18 or 22, or encoding a polypeptide with at least 70% identity to SEQ ID NO 26 or 30.

In various embodiments, the lantibiotic peptide or bacteriocin is a Class I or Class II lantibiotic peptide or bacteriocin.

In various embodiments, the lantibiotic peptide is salA, salB, sal9 or a combination thereof.

In various embodiments, the lantibiotic peptide is salA, salB, or a combination thereof.

In various embodiments, wherein the bacteriocin is salQ.

In various embodiments, the method increases production of a polypeptide with at least 70% sequence identity to any one of SEQ ID NOs 23-30.

In various embodiments, at the polypeptide has at least 75% identity to any one of SEQ ID NOs 23-30, preferably at least 80%, 85%, 90%, 95%, or 99% identity to any one of SEQ ID NOs 23-30.

In various embodiments, the method increases the inhibitory profile of S. salivarius against skin, dental, oral, mucosal and/or ENTR microorganisms.

In various embodiments, the skin, oral, dental, mucosal, and/or ENTR microorganism is selected from Staphylococcus aureus spp., Staphylococcus intermedius spp., Staphylococcus saprophyticus spp., Moraxella catarrhalis spp., Haemophilus influenzae spp., Streptococcus pyogenes spp., Pseudomonas aeruginosa spp., Streptococcus mutans spp., Streptococcus pneumoniae spp., Cutibacterium acnes, Candida albicans spp. Streptococcus sobrinus spp., Corynebacterium spp., Fusobacterium nucleatum spp., Aggregatibacter actinomycetemcomitans spp., Porphyromonas gingivalis spp., Tannerella forsythia spp., Treponema denticola spp., P. intermedia spp., Prevotella spp., Actinomyces viscosus spp., Streptococcus equismillis spp., Streptococcus dygalactiae spp., Streptococcus sanguis spp., Staphylococcus cohnii spp., B. intermedius spp., Atopobium parvulum spp., Eubacterium saburreum spp., Eubacterium sulci spp., Parvimonas micra spp., Solobacterium moorei spp., Streptococcus agalactiae spp., C. minutissimus spp., Propionibacterium propionicus spp., Streptococcus agalactiae spp., Streptococcus dysgalactiae spp., Staphylococcus simulans spp., Staphylococcus xylosus spp., Tinea pedis infection causing fungi, S. salivarius spp. other than K12 or M18, Lactococcus lactis spp., Staphylococcus epidermidis spp., Streptococcus constellatus spp., Klebsiella pneumoniae spp., Acinetobacter baumanii spp. or any combination of any two or more thereof.

In various embodiments, the microorganism is selected from S. aureus A222, S. aureus 20, S. aureus 14, S. aureus 19, S. aureus A504, S. saprophyticus ATCC 15305, M. catarrhalis TW1, M. catarrhalis TW2, H. influenzae TW5, S. pyogenes M76, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, P. aeruginosa 12, S. mutans OMZ175, S. pneumoniae D39, L. lactis T-21, S. epidermidis 11, S. constellatus T-29, S. salivarius 6, S. salivarius 193, S. salivarius 20P3, or any combination of any two or more thereof.

In various embodiments, the composition comprises at least about 0.1% by weight of S. salivarius.

In various embodiments, the composition comprises from about 0.1 to about 20% by weight of S. salivarius.

In various embodiments, the composition comprises at least about 1×10³ cfu/g of S. salivarius.

In various embodiments, the composition comprises from about 1×10³ to about 1×10¹³ cfu/g of S. salivarius.

In various embodiments, the composition comprises less than about 20% by weight supplemental saccharide.

In various embodiments, the composition comprises from about 0.1 to about 20% by weight supplemental saccharide.

In various embodiments, the composition is formulated for oral, dental, nasal, mucosal, topical, or pulmonary administration.

In various embodiments, the composition is formulated in a slow-release composition.

In various embodiments, the composition is formulated into a powder, lozenge, nasal spray, nasal gel, nasal drop, oral drop, oral gel, oral spray, inhalable, topical composition, chewable, melt, film, gummy, toothpaste, tooth-gel, varnish, mousse, mouthwash, food product (e.g. yoghurt), cream, gel spray, deodorant, serum, lotion, balm, moisturiser, pessary, or suppository.

In various embodiments, the microorganism is a Streptococcus or Staphylococcus bacteria selected from S. aureus spp., S. saprophyticus spp., S. mutans spp., S. pyogenes spp., S. pneumoniae spp.; and the S. salivarius strain is K12.

In various embodiments, the Streptococcus or Staphylococcus bacteria is selected from S. aureus A222, S. saprophyticus ATCC 15305, S. mutans OMZ175, S. constellatus T-29, S. pyogenes 71-698, and S. pneumoniae D39; and the S. salivarius strain is K12.

In various embodiments, the supplemental saccharide is raffinose and is present in the composition in an amount of 0.5 to 15%, or 1 to 12%, or 1.5 to 10%, or 2 to 7%, or 2.5 to 5% by weight.

In various embodiments, the supplemental saccharide is galactose and is present in the composition in an amount of 0.5 to 15%, or 1 to 12%, or 1.5 to 10%, or 2 to 7%, or 2.5 to 5% by weight.

In various embodiments, the bacteria are selected from S. pyogenes spp., and S. pneumoniae spp.; and the S. salivarius strain is M18.

In various embodiments, the bacteria are selected from S. pyogenes 71-698, and S. pneumoniae D39; and the S. salivarius strain is M18.

In various embodiments, the bacteria are selected from S. constellatus, S. mutans, and S. saprophyticus; and the S. salivarius strain is M18.

In various embodiments, the bacteria are selected from S. constellatus T29, S. mutans OMZ175, and S. saprophyticus ATCC 15305; and the S. salivarius strain is M18.

In various embodiments, the supplemental saccharide is raffinose and is present in the composition in an amount of 0.25 to 10%, or 0.5 to 8%, or 0.75 to 7%, or 1 to 6%, or 1.25 to 5% by weight.

In various embodiments, the supplemental saccharide is galactose and is present in the composition in an amount of 0.25 to 10%, or 0.5 to 8%, or 0.75 to 7%, or 1 to 6%, or 1.25 to 5% by weight.

In various embodiments, the composition comprises one or more of: galactose in an amount of 0.1 to 1%, or 0.2 to 0.8, or 0.25 to 0.75, or at 0.5% by weight, and raffinose in an amount of 0.5 to 5%, or 1 to 4, or 2 to 3, or 2.5% by weight.

In various embodiments, the composition comprises one or more of: galactose in an amount of about 1.7% by weight; and raffinose in an amount of about 1.25% by weight.

In various embodiments, the composition comprises one or more of: galactose in an amount of about 0.5% by weight; and raffinose in an amount of about 2.5% by weight.

In various embodiments, the composition comprises raffinose in an amount of 2.0, 2.1, 2.2, 2.3, 2.4, 2.5, 2.6, 2.7, 2.8, 2.9, or 3.0% by weight.

In various embodiments, the composition comprises galactose in an amount of 0.1, 0.2, 0.3, 0.4, 0.5, 0.6, 0.7, 0.8, 0.9, 1.0% by weight.

In various embodiments, the composition comprises raffinose in an amount of 1.2, 1.3, 1.4, 1.5, 1.6, 1.7, 1.8, 1.9, 2.0, 2.1, or 2.2% by weight.

In various embodiments, the composition comprises galactose in an amount of 0.7, 0.8, 0.9, 1.0, 1.1, 1.2, 1.25, 1.3, 1.4, 1.5, 1.6 or 1.7% by weight.

In various embodiments, the composition further comprises one or more of a carrier; a tableting aid, including a binder or a lubricant; and a flavouring agent.

In various embodiments, the therapeutic formulation is formulated for oral, dental, nasal, mucosal, topical, or pulmonary administration.

In various embodiments, the therapeutic formulation is a slow-release composition.

In various embodiments, the therapeutic formulation is a powder, lozenge, nasal spray, nasal gel, nasal drop, oral drop, oral gel, oral spray, inhalable, topical composition, chewable, melt, film, gummy, toothpaste, tooth-gel, varnish, mousse, mouthwash, food product (e.g. yoghurt), cream, gel, spray, deodorant, serum, lotion, balm, moisturiser, pessary, or suppository.

In various embodiments, the therapeutic formulation is a powder.

In various embodiments, the therapeutic formulation is a lozenge.

In various embodiments, the disease or disorder is caused by an oral, dental, mucosal, skin, or ENTR pathogen.

In various embodiments, the disease or disorder is caused by a pathogenic Streptococcus or Staphylococcus bacteria.

In various embodiments, the pathogenic Streptococcus or Staphylococcus bacteria is selected from S. aureus spp., S. saprophyticus spp., S. mutans spp., S. pyogenes spp., and S. pneumoniae spp.

In various embodiments, the disease or disorder is selected from otitis media, sore throat, tooth decay, acute pharyngitis, tonsillitis, pneumonia, chronic obstructive pulmonary disease (COPD), periodontal disease, gingivitis, halitosis, dental caries, sepsis, meningitis, candidiasis (oral thrush), vaginitis, body odour, acne, actinomycosis, psoriasis, erythrasma, cellulitis, impetigo, atopic dermatitis, bacteraemia, athlete's foot, soft tissue infections, erythema, nosocomial, erythema, SARS-CoV, influenza A, influenza B, and RSV or any combination of any two or more thereof.

In various embodiments, the microorganism sensitive to Blis-producing S. salivarius is selected from S. salivarius spp., S. epidermidis spp., S. constellatus spp., and L. lactis spp.

In various embodiments, the microorganism sensitive to Blis-producing S. salivarius is selected from S. pyogenes spp., F. nucleatum spp., and P. gingivalis spp.

In various embodiments, the subject is a human. In some embodiments, the human is a child.

In various embodiments, the composition is a cosmetic.

In various embodiments, the composition is a dietary supplement.

In various embodiments, the composition is a natural health product.

In various embodiments, the composition is a complementary medicine.

In various embodiments, the composition is a lozenge, and the method for manufacturing the composition further comprises a step of pressing the homogeneous blend to produce the lozenge.

In various embodiments, the inhibitory profile of the S. salivarius in the composition is improved relative to a composition lacking the supplemental saccharide.

In various embodiments, the composition is for use in:
(a) the treatment or prevention of a disease or disorder, or
(b) the inhibition of a microorganism sensitive to Blis-producing S. salivarius.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The present invention will be described with reference to the accompanying figures, in which:

FIG. 1 shows the change in the size of ENTR pathogen ZOI size (mm) following a deferred antagonism assay conducted with K12 as the producer organism cultured in the presence of various concentrations of galactose. The control condition was set to (normalised) 0 mm, any deviation from 0 represents the change in the ZOI size (mm).

FIG. 2 shows the change in the size of ENTR pathogen ZOI size (mm) following a deferred antagonism assay conducted with K12 as the producer organism cultured in the presence of various concentrations of raffinose. The control condition was set to 0 mm, any deviation from 0 represents the change in the ZOI size (mm).

FIG. 3 shows the change in the size of ENTR pathogen ZOI size (mm) following a deferred antagonism assay conducted with M18 as the producer organism cultured in the presence of various concentrations of galactose. The control condition was set to 0 mm, any deviation from 0 represents the change in the ZOI size (mm).

FIG. 4 shows the change in the size of ENTR pathogen ZOI size (mm) following a deferred antagonism assay conducted with M18 as the producer organism cultured in the presence of various concentrations of raffinose. The control condition was set to 0 mm, any deviation from 0 represents the change in the ZOI size (mm).

FIG. 5 shows the comparison of stimulation on K12 inhibitory effect using raffinose vs saccharides (equimolar) against skin pathogen S. aureus A222. Raffinose vs (a) Trimix (mixture of equimolar concentrations of the three saccharides galactose, glucose and fructose present in raffinose); (b) individual saccharides; (c) comparison of stimulation on M18 inhibitory effect using raffinose vs saccharides (equimolar) against skin pathogen S. aureus A222. Raffinose vs individual saccharides.

FIG. 6 shows the comparison of stimulation on K12 inhibitory effect using raffinose vs saccharides (equimolar) against dental pathogen S. mutans OMZ175. Raffinose vs (a) Trimix (mixture of equimolar concentrations of the three saccharides); (b) individual saccharides; (c) comparison of stimulation on M18 inhibitory effect using raffinose vs saccharides (equimolar) against dental pathogen S. mutans OMZ175. Raffinose vs individual saccharides.

FIG. 7 shows the comparison of stimulation on K12 inhibitory effect using raffinose vs saccharides (equimolar) against Lower respiratory tract pathogen S. pneumoniae D39. Raffinose vs (a) Trimix (mixture of equimolar concentrations of the three saccharides); (b) individual saccharides; (c) comparison of stimulation on M18 inhibitory effect using raffinose vs saccharides (equimolar) against Lower respiratory tract pathogen S. pneumoniae D39. Raffinose vs individual saccharides.

FIG. 8 shows the comparison of stimulation on K12 inhibitory effect using raffinose vs saccharide (equal percentage weight) against Skin pathogen S. aureus A222. Raffinose vs (a) Trimix (mixture of equal weight percentage concentrations of the three saccharides); (b) individual saccharides; (c) comparison of stimulation on M18 inhibitory effect using raffinose vs saccharides (equal weight percentage) against skin pathogen S. aureus A222. Raffinose vs individual saccharides.

i 16] FIG. 9 shows the comparison of stimulation on K12 inhibitory effect using raffinose vs saccharides (equal percentage weight) against Dental pathogen S. mutans OMZ175. Raffinose vs (a) Trimix (mixture of equal weight percentage concentrations of the three saccharides); (b) individual saccharides; (c) comparison of stimulation on M18 inhibitory effect using raffinose vs saccharides (equal weight percentage) against Dental pathogen S. mutans OMZ175. Raffinose vs individual saccharides.

FIG. 10 shows the comparison of stimulation on K12 inhibitory effect using raffinose vs saccharides (equal percentage weight) against ENTR pathogen S. pyogenes 71-968. Raffinose vs (a) Trimix (mixture of equal weight percentage concentrations of the three saccharides); (b) individual saccharides; (c) comparison of stimulation on M18 inhibitory effect using raffinose vs saccharides (equal weight percentage) against ENTR pathogen S. pyogenes 71-968. Raffinose vs individual saccharides.

FIG. 11 shows the comparison of stimulation on K12 inhibitory effect using raffinose vs saccharides (equal percentage weight) against microorganism sensitive to Blis-producing S. salivarius; S. constellatus T29. Raffinose vs (a) Trimix (mixture of equal weight percentage concentrations of the three saccharides); (b) individual saccharides; (c) comparison of stimulation on M18 inhibitory effect using raffinose vs saccharides (equal weight percentage) against microorganism sensitive to Blis-producing S. salivarius; S. constellatus T29. Raffinose vs individual saccharides.

FIG. 12 shows the comparison of stimulation on K12 inhibitory effect using raffinose vs saccharides (equal percentage weight) against lower respiratory tract pathogen S. pneumoniae D39. Raffinose vs (a) Trimix (mixture of equal weight percentage concentrations of the three saccharides); (b) individual saccharides; (c) comparison of stimulation on M18 inhibitory effect using raffinose vs saccharides (equal weight percentage) against lower respiratory tract pathogen S. pneumoniae D39. Raffinose vs individual saccharides.

FIG. 13 shows the comparison of stimulation on K12 inhibitory effect using raffinose vs saccharides (equal percentage weight) against ENTR pathogens S. pyogenes 71-698, dental pathogen S. mutans OMZ175, S. pneumoniae D39, skin pathogens S. saprophyticus ATCC 15305 and S. aureus A222 and microorganism sensitive to Blis-producing S. salivarius; S. constellatus T29. Raffinose vs Trimix (mixture of equal weight percentage concentrations of the three saccharides) and individual saccharides. Raffinose vs (a) Trimix (mixture of equal weight percentage concentrations of the three saccharides); (b) individual saccharides; (c) comparison of stimulation on M18 inhibitory effect using raffinose vs saccharides (equal weight percentage) against range of pathogens and microorganism sensitive to Blis-producing S. salivarius. Raffinose vs individual saccharides.

FIG. 14 shows the comparison of stimulation on K12 inhibitory effect using raffinose vs saccharides (equal percentage weight) against microorganism sensitive to Blis-producing S. salivarius; S. salivarius #6, S. salivarius 193, S. salivarius 20P3. Raffinose vs Trimix (mixture of equal weight percentage concentrations of the three saccharides); (b) individual saccharides.

FIG. 15 shows the comparison of stimulation on M18 inhibitory effect using raffinose vs saccharides (equal percentage weight) against microorganism sensitive to Blis-producing S. salivarius; S. salivarius #6, S. salivarius 193, S. salivarius 20P3. Raffinose vs Trimix (mixture of equal weight percentage concentrations of the three saccharides) and individual saccharides.

FIG. 16 shows the comparison of stimulation on K12 inhibitory effect using raffinose vs saccharides (equal percentage weight) against several oral and ENTR pathogens S. pyogenes 71-698, S. pyogenes FF22, S. pyogenes 71-679, S. pyogenes W-1, S. pyogenes M17, S. pyogenes M57, S. pyogenes EMM92, S. pyogenes M66 and S. pyogenes M74. Raffinose vs Trimix (mixture of equal weight percentage concentrations of the three saccharides) and individual saccharides.

FIG. 17 shows the comparison of stimulation on M18 inhibitory effect using raffinose vs saccharides (equal percentage weight) against several oral and ENTR pathogens S. pyogenes 71-698, S. pyogenes FF22, S. pyogenes 71-679, S. pyogenes W-1, S. pyogenes M17, S. pyogenes M57, S. pyogenes EMM92, S. pyogenes M66 and S. pyogenes M74. Raffinose vs Trimix (mixture of equal weight percentage concentrations of the three saccharides) and individual saccharides.

FIG. 18 shows the comparison of stimulation on K12 inhibitory effect using raffinose vs saccharides (equal percentage weight) against several lower respiratory tract pathogens S. dysgalactiae Bris 2, S. dysgalactiae T277, S. pneumoniae D39, S. pneumoniae RX1, S. pneumoniae PK8. Raffinose vs Trimix (mixture of equal weight percentage concentrations of the three saccharides) and individual saccharides.

FIG. 19 shows the comparison of stimulation on M18 inhibitory effect using raffinose vs saccharides (equal percentage weight) against several ENTR/lower respiratory tract pathogens S. dysgalactiae Bris 2, S. dysgalactiae T277, S. pneumoniae D39, S. pneumoniae RX1, S. pneumoniae PK8. Raffinose vs Trimix (mixture of equal weight percentage concentrations of the three saccharides) and individual saccharides.

FIG. 20 shows the comparison of stimulation on K12 inhibitory effect using raffinose vs saccharides (equal percentage weight) against several dental pathogens S. mutans OMZ175, S. mutans ATCC 10449, S. mutans D10, S. mutans UA159, S. mutans FW75, A. viscosus T14, S. sanguis K11, S. sobrinus OMZ176. Raffinose vs Trimix (mixture of equal weight percentage concentrations of the three saccharides) and individual saccharides.

FIG. 21 shows the comparison of stimulation on M18 inhibitory effect using raffinose vs saccharides (equal percentage weight) against several dental pathogens S. mutans OMZ175, S. mutans ATCC 10449, S. mutans D10, S. mutans UA159, S. mutans FW75, A. viscosus T14, S. sanguis K11, S. sobrinus OMZ176. Raffinose vs Trimix (mixture of equal weight percentage concentrations of the three saccharides) and individual saccharides.

FIG. 22 shows the comparison of stimulation on K12 inhibitory effect using raffinose vs saccharides (equal percentage weight) against several skin pathogens S. cohnii, S. simulans, S. aureus A222, S. aureus 20, S. aureus 19, S. aureus 14. Raffinose vs Trimix (mixture of equal weight percentage concentrations of the three saccharides) and individual saccharides.

FIG. 23 shows the comparison of stimulation on M18 inhibitory effect using raffinose vs saccharides (equal percentage weight) against several skin pathogens S. cohnii, S. simulans, S. aureus A222, S. aureus 20, S. aureus 19, S. aureus 14. Raffinose vs Trimix (mixture of equal weight percentage concentrations of the three saccharides) and individual saccharides.

FIG. 24 shows the comparison of stimulation on dairy free K12 inhibitory effect using raffinose vs saccharides (equal percentage weight) against ENTR pathogens S. pyogenes 71-S. pneumoniae D39698, dental pathogen S. mutans OMZ175, skin pathogens S. saprophyticus ATCC 15305 and S. aureus A222 and microorganism sensitive to Blis-producing S. salivarius; S. constellatus T29. Raffinose vs Trimix (mixture of equal weight percentage concentrations of the three saccharides) and individual saccharides.

FIG. 25 shows the comparison of stimulation on dairy free M18 inhibitory effect using raffinose vs saccharides (equal percentage weight) against ENTR pathogens S. pyogenes 71-698, S. pneumoniae D39, dental pathogen S. mutans OMZ175, skin pathogens S. saprophyticus ATCC 15305 and S. aureus A222 and microorganism sensitive to Blis-producing S. salivarius; S. constellatus T29. Raffinose vs Trimix (mixture of equal weight percentage concentrations of the three saccharides) and individual saccharides.

FIG. 26 shows the comparison of stimulation of growth of K12 in M17 broth. Raffinose, galactose, Trimix (mixture of equal weight percentage concentrations of the three saccharides) and individual saccharides.

FIG. 27 shows the comparison of stimulation of growth of K12 in M17 broth and inhibitory effect using effect using raffinose vs saccharides (equal percentage weight) against oral and ENTR pathogens S. pyogenes 71-698, S. pyogenes FF22, S. pyogenes 71-679, S. pyogenes W-1, S. pyogenes M17, S. pyogenes M57, S. pyogenes EMM92, S. pyogenes M66 and S. pyogenes M74. Raffinose vs Trimix (mixture of equal weight percentage concentrations of the three saccharides) and individual saccharides.

FIG. 28 shows the comparison of stimulation of growth of K12 in M17 broth and inhibitory effect using effect using raffinose vs saccharides (equal percentage weight) against ENTR pathogens S. pyogenes 71-698, S. pyogenes M74, S. pyogenes M66, S. pneumoniae D39, dental pathogen S. mutans OMZ175, skin pathogens S. saprophyticus ATCC 15305 and S. aureus A222 and microorganism sensitive to Blis-producing S. salivarius; S. constellatus T-29. Raffinose vs Trimix (mixture of equal weight percentage concentrations of the three saccharides) and individual saccharides glucose, fructose and galactose.

FIG. 28A shows the comparison of stimulation of growth of M18 in M17 broth. Raffinose, Galactose, Trimix (mixture of equal weight percentage concentrations of the three saccharides) and individual saccharides.

FIG. 28B shows the comparison of stimulation of growth of M18 in M17 broth and inhibitory effect using effect using raffinose vs saccharides (equal percentage weight) against ENTR pathogens S. pyogenes 71-698, S. pneumoniae D39, dental pathogen S. mutans OMZ175, skin pathogen S. saprophyticus ATCC 15305 and microorganism sensitive to Blis-producing S. salivarius; S. constellatus T-29. Raffinose vs Trimix (mixture of equal weight percentage concentrations of the three saccharides) and individual saccharides glucose, fructose and galactose.

FIG. 29 shows magnified photos of K12 and M18 mucoid morphology when grown in CABCa agar supplemented with raffinose compared to CABCa control.

FIG. 30 shows photos of K12 and M18 (dairy or dairy free) change to mucoid morphology when grown in CABCa agar supplemented with raffinose compared to CABCa control.

FIG. 31 shows photos of K12 and M18 producer streaks and glass slides showing bacterial growth. Dotted black ovals visually shows that a higher amount of mucous is produced in CABCa agar supplemented with raffinose compared to CABCa supplemented with Trimix (mixture of equal weight percentage concentrations of the three saccharides) and CABCa control.

FIG. 32 shows the effect of 2.5% w/w raffinose on the antimicrobial activity of different S. salivarius strains against ENTR pathogens S. pyogenes 71-698, S. pneumoniae D39, dental pathogens S. sobrinus OMZ176, S. mutans OMZ175, skin pathogens S. saprophyticus ATCC 15305 and S. aureus A222 and microorganism sensitive to Blis-producing S. salivarius; S. constellatus T29.

FIG. 33 shows the effect of 0.5% w/w galactose on the antimicrobial activity of different S. salivarius strains against ENTR pathogens S. pyogenes 71-698, S. pneumoniae D39, dental pathogens S. sobrinus OMZ176, S. mutans OMZ175, skin pathogens S. saprophyticus ATCC 15305 and S. aureus A222 and microorganism sensitive to Blis-producing S. salivarius; S. constellatus T29.

FIG. 34 shows the effect of raffinose, galactose and their combination on the antimicrobial activity of S. salivarius K12 against different gram-negative strains F. nucleatum ATCC 25586, F. nucleatum FH2, F. nucleatum FH3, P. gingivalis ATCC 33277, P. gingivalis W50, P. intermedia ATCC 23611, pathogens implicated in Halitosis.

FIG. 35 shows the effect of raffinose, galactose and their combination on the antimicrobial activity of S. salivarius M18 against different gram-negative strains F. nucleatum ATCC 25586, F. nucleatum FH2, F. nucleatum FH3, P. gingivalis ATCC 33277, P. gingivalis W50, P. intermedia ATCC 23611, pathogens implicated in Halitosis.

FIG. 36 shows effect of raffinose, galactose and their combination on the antimicrobial activity of S. salivarius K12 freeze dried raw ingredient against ENTR pathogens S. pyogenes 71-698, S. pneumoniae D39, dental pathogens S. sobrinus OMZ176, S. mutans OMZ175, skin pathogens S. saprophyticus ATCC 15305 and S. aureus A222 and microorganism sensitive to Blis-producing S. salivarius; S. constellatus T29.

FIG. 37 shows the effect of raffinose, galactose and their combination on the antimicrobial activity of S. salivarius M18 freeze dried raw ingredient against ENTR pathogens S. pyogenes 71-698, S. pneumoniae D39, M. catarrhalis TW1, dental pathogens S. sobrinus OMZ176, S. mutans OMZ175, skin pathogens S. saprophyticus ATCC 15305 and S. aureus A222 and microorganism sensitive to Blis-producing S. salivarius; S. constellatus T29.

FIG. 38 shows the effect of raffinose, galactose and their combination on the antimicrobial activity of a combination of S. salivarius K12 and S. salivarius M18 freeze dried raw ingredient against ENTR pathogens S. pyogenes 71-698, S. pneumoniae D39, S. agalactiae ATCC 12386, M catarrhalis TW1, dental pathogens S. sobrinus OMZ176, S. mutans OMZ175, skin pathogens S. saprophyticus ATCC 15305 and S. aureus A222 and microorganisms sensitive to Blis-producing S. salivarius; S. constellatus T29 and L. lactis ATCC19435.

FIG. 39 shows the effect of supplemental saccharide on the inhibitory activity of S. salivarius K12 against pathogens S. pyogenes 71-698, S. pyogenes 71-679, S. pyogenes H13, S. pyogenes K26, S. pyogenes H29, S. pyogenes WS02, S. mutans OMZ175, S. mutans FW75, S. sobrinus ATCC 27351, S. sanguis K11, S. agalactiae ATCC 12386, A. viscosus T14, A. viscosus ATCC 15987, S. constellatus T29, S. pyogenes K7, S. pneumoniae D39, S. pneumoniae PK8, S. sobrinus OMZ176, S. dysgalactiae T-148, S. dysgalactiae Bris 2, S. dysgalactiae T277, C. auris ATCC 51966, S. aureus 19, in a commercial powder formulation (Split plate method).

FIG. 40 shows a comparison of antimicrobial activity of five compositions: S. salivarius K12-containing commercial powder dosage form (Daily Defense Junior) combined with 0.5% w/w galactose; Daily Defense Junior combined with 2.5% w/w raffinose; Daily Defence Junior combined with 0.5% w/w galactose and 2.5% w/w raffinose; a commercial infant formula combined with S. salivarius K12; whole milk powder combined with S. salivarius K12.

FIG. 41 shows the relative expression of salA in S. salivarius K12 with the following saccharides added to the solid medium (CABCa agar); 0.5% w/w galactose; 2.5% w/w raffinose; 0.5% w/w galactose in combination with 2.5% w/w raffinose; 0.5% w/w sucrose; 2.5% w/w sucrose. Data points are means (n=3±SD). Data points are means (n=3±SD).

FIG. 42 shows the relative expression of salB in S. salivarius K12 when different combinations of sugars are added to the solid medium (CABCa agar); 0.5% w/w Galactose; 2.5% w/w raffinose; 0.5% w/w galactose in combination with 2.5% w/w raffinose; 0.5% w/w sucrose; 2.5% w/w sucrose. Data points are means (n=3±SD).

FIG. 43 shows the relative expression of salQ in S. salivarius K12 when different combinations of supplemental saccharides are added to the solid medium (CABCa agar); 0.5% w/w Galactose; 2.5% w/w raffinose; 0.5% w/w galactose in combination with 2.5% w/w raffinose; 0.5% w/w sucrose; 2.5% w/w sucrose. Data points are means (n=3±SD).

FIG. 44 shows the relative expression of Urease (ureC) in S. salivarius K12 when different combinations of supplemental saccharides are added to the solid medium (CABCa agar); 0.5% w/w galactose; 2.5% w/w raffinose; 0.5% w/w Galactose in combination with 2.5% w/w raffinose; 0.5% w/w sucrose; 2.5% w/w sucrose. Data points are means (n=3±SD).

FIG. 45 shows the average percentage of S. salivarius K12 (of total S. salivarius) in saliva samples obtained from the groups of participants using lozenges comprising S. salivarius K12 without supplemental saccharides (G1), with galactose (G2), raffinose (G3) and raffinose and galactose (G4) at 1 hours, 8 hours and 24 hours after dissolving first lozenge in the mouth, and at 48 hours after a 7-day period of daily dissolving a lozenge in the mouth.

### DETAILED DESCRIPTION OF THE INVENTION

The role of saccharides, and in particular digestible saccharides such as mono-, di- and tri-saccharides for stimulating growth and/or activity of oral bacteria has not been fully explored. The inventors sought to better understand the role of such saccharides on oral probiotic performance.

The metabolism of certain simple sugars, such as glucose, can act to supress the activation/transcription of certain genes including bacteriocin genes in some bacteria. This effect is commonly known as catabolite repression (see: Görke & Stülke, Nature Reviews Microbiology (2008) 6, 613-624). While assessing whether other sugars had a similar repressive effect, the inventors surprisingly found that the supplementation of the growth medium with certain other saccharides (such as lactose) had the opposite effect, enhancing the inhibitory spectrum of some strains of bacterial species, including but not limited to Streptococcus salivarius.

### Definitions

The following definitions are presented to better define the present invention and as a guide for those of ordinary skill in the art in the practice of the present invention. Unless otherwise specified, all technical and scientific terms used herein are to be understood as having the same meanings as is understood by one of ordinary skill in the relevant art to which this disclosure pertains.

The general chemical and biological terms used, for example, in the formulae herein have their usual meanings.

Examples of definitions of common terms in microbiology, molecular biology and biochemistry can be found in Methods for General and Molecular Microbiology, 3^{rd} Edition, C. A. Reddy, et al. (eds.), ASM Press, (2008); Encyclopaedia of Microbiology, 2nd ed., Joshua Lederburg, (ed.). Academic Press, (2000); Microbiology By Cliffs Notes, I. Edward Alcamo, Wiley, (1996); Dictionary of Microbiology and Molecular Biology, Singleton et al. (2d ed.) (1994); Biology of Microorganisms 11 hed. Brock et al., Pearson Prentice Hall, (2006); Biodiversity of Fungi: Inventory and Monitoring Methods, Mueller et al. Academic Press, (2004); Genes IX, Benjamin Lewin, Jones & Bartlett Publishing, (2007); The Encyclopaedia of Molecular Biology, Kendrew et al. (eds.), Blackwell Science Ltd., (1994); and Molecular Biology and Biotechnology: a Comprehensive Desk Reference, Robert A. Meyers (ed.), VCH Publishers, Inc., (1995). These sources also provide standard microbiological, molecular biology and biochemistry protocols and procedures that can be used to perform the present invention.

The term “comprising” as used in this specification and claims means “consisting at least in part of”. When interpreting each statement in this specification and claims that includes the term “comprising”, features other than that or those prefaced by the term may also be present. Related terms such as “comprise”, “comprised” and “comprises” are to be interpreted in the same manner.

As used herein the term “and/or” means “and” or “or”, or both.

As used herein “(s)” following a noun means the plural and/or singular forms of the noun.

The term “subject” as used herein refers to an animal, including humans, and non-human animals such as dogs, pigs, cats, horses, sheep, cows, chickens, fish, and other domestic and farm animals.

The term “treatment” as used herein refers to a subject undergoing prophylactic or therapeutic treatment. Prophylactic treatment includes treatment to prevent or reduce the likelihood or severity of infection, or to inhibit or control a microbial population. Treatment can be to inhibit or reduce a microbial population in a subject. Treatment can also be provided to an infected subject to decrease the severity of, or to reduce or eliminate an infection or its associated symptoms. The subject to be treated may be at any age, e.g., infant, childhood, adolescence, adulthood, or elderly. Where a “patient,” “individual,” or “host” is referred to it is synonymous with the term “subject”.

As used herein, a “supplemental saccharide” is a saccharide that improves the inhibitory profile and/or mucoid properties of a bacterial species, such as S. salivarius.

As used herein “improving the inhibitory profile of Streptococcus salivarius” means enhancing the potency of inhibition, and/or increasing the spectrum of inhibitory activity of the S. salivarius compared to the S. salivarius in the absence of an effective amount of a supplemental saccharide. The improvement is observed whether the supplemental saccharide is added to a composition comprising other saccharides, or a composition which is saccharide free.

As used herein the term “upregulating a gene or genes” means increasing expression levels of the transcribed RNA encoded by the gene resulting in increased peptide, bacteriocin or protein. The related term “a method of upregulating a gene” means a method of increasing expression levels of the transcribed RNA encoded by the gene resulting in increased peptide, bacteriocin or protein. In embodiments, the method results in increase of expression levels of the transcribed RNA encoded by the gene resulting in increased expression of said peptide, bacteriocin or protein, and/or increase of production of the said peptide, bacteriocin or protein.

As used herein “improving the mucoid properties” of an S. salivarius species means bacterial colonies of the S. salivarius are more mucoid (sticky mucus-like) making the probiotic more capable of adhering to substrates and thus allowing for enhanced colonisation.

An “ENTR” microorganism is an ear, nose, throat, or respiratory tract microorganism and includes pathogens that infect the ear (aural), nose (nasal), throat, upper respiratory tract, and lower respiratory tract, and non-pathogenic microorganisms that colonise the ear (aural), nose (nasal), throat, upper respiratory tract, and lower respiratory tract. The term “lower respiratory tract” or “LRT” as used herein means the trachea, bronchi, and lungs. The term is contrasted with the “upper respiratory tract” or “URT” which means nose, nasal passages, and nasopharynx.

A “salivaricin” is a bacteriocin-like inhibitory substance (BLIS) produced by a S. salivarius strain. Accordingly, a Blis-producing S. salivarius is an S. salivarius which produces a salivaricin.

The term “microbe” or “microorganism” as used herein refers to a bacteria, fungus, virus or a combination thereof. Similarly, the term “microbial” population, infection, disease or condition as used herein includes bacterial, viral and fungal populations, infections, diseases, or conditions. A microorganism may be pathogenic, or non-pathogenic (e.g. commensal).

The term an “effective amount” as used herein means an amount effective to inhibit, reduce or control a microbial population, or to protect against, delay, reduce, stabilise, improve or treat a microbial infection in and/or on a patient. It also refers to an amount sufficient to provide a beneficial effect to a patient. Such beneficial effects may include detectable: increase in IFN-γ, reduction in NF-kB-mediated cytokine response in lungs, inhibition of microbial replication, and/or decrease in microbial load. The effect should be sufficient to provide a medically significant decrease in the likelihood of a bacterial, fungal or viral infection, or a medically or statistically significant decrease in the rate, extent, severity, or length of a microbial infection, or associated symptoms, or secondary infections. Reduction in the survival, growth and/or proliferation of the microorganism is contemplated.

In one embodiment the term “statistically significant” as used herein refers to the likelihood that a result or relationship is caused by something other than random chance. A result may be found to be statistically significant using statistical hypothesis testing as known and used in the art. Statistical hypothesis testing provides a “P-value” as known in the art, which represents the probability that the measured result is due to random chance alone. It is believed to be generally accepted in the art that levels of significance of 5% (0.05) or lower are considered to be statistically significant.

The phrase “inhibiting the growth of a microorganism sensitive to a composition of the invention” and like terms refer to the growth inhibition of at least one or more species of microorganism sensitive to a BLIS-producing streptococcal strain, e.g., S. salivarius. Inhibition of bacterial growth can be determined by a variety of methods including inhibition of colony forming units (CFU) of a targeted bacterial strain as described in WO01/27143. Inhibition of viral growth may be determined by a variety of methods but can include the Virus Yield Reduction Assay (VYR), observation of cytopathic effect or virucidal assays. Inhibition of fungal growth can be determined e.g. by adaption for fungi of the deferred antagonism method (Tagg and Bannister 1979; Med Microbiology 12:397) as described in the examples. Inhibition of germ tube formation provides another measure of anti-fungal activity (e.g. Reynolds and Braude (1956) Clin Res Proc 4:40).

The term “contacting” as used herein refers to both direct and indirect contact between the microorganism and a Blis composition. Indirect contact comprises exposure of the microorganism in its environment, particularly native environment, to a Blis composition.

A “composition” or “Blis composition” as used herein refers to a composition or formulation comprising S. salivarius K12, M18 or combination thereof, and may include BLIS-containing naturally-released extracellular products thereof such as salivaricins or other antimicrobial agents; and optionally a carrier, diluent or excipient. In some embodiments, the composition can be a cosmetic, a dietary supplement, a natural health product, or a complementary medicine. A dietary supplement (also known as a food supplement) is a foodstuff that is intended to supplement the normal diet and contains concentrated nutrients, that is vitamins or minerals, or other substances with a nutritional or physiological effect. A natural health product includes probiotics, herbal remedies, vitamins and minerals, homeopathic medicines, traditional medicines, and amino acids and essential fatty acids. A complementary medicine is a medicine which has been evaluated for safety and quality, and may have been evaluated for efficacy.

The unit “cfu/g” means colony-forming units per gram.

As used herein, the term “variant” refers to gene, polynucleotide, or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues.

In certain embodiments, variants of the polynucleotides and polypeptides disclosed herein possess biological activities that are the same or similar to those of the polynucleotides or polypeptides disclosed herein. The term “variant” with reference to polynucleotides and polypeptides encompasses all forms of polynucleotides and polypeptides as defined herein.

### Method of the Invention

In a first aspect, the invention provides a method of improving the inhibitory profile of S. salivarius comprising formulating the S. salivarius in a composition comprising an effective amount of a supplemental saccharide, wherein the Streptococcus salivarius is S. salivarius M18, S. salivarius K12, or a combination thereof, and wherein the supplemental saccharide is galactose, or raffinose, or a combination thereof.

Potency of inhibition is the extent to which an inhibited microorganism is inhibited. Potency of inhibition may be measured by determining the size of the zone of inhibition (ZOI) compared to a control sample on an agar plate, as described in Tagg J R, Bannister LV. “Fingerprinting” beta-haemolytic streptococci by their production of and sensitivity to bacteriocine-like inhibitors. J Med Microbiol. 1979 November; 12(4):397-411. A more potent inhibitory effect will be evident by a larger zone of inhibition. Another method of determining potency of inhibition is a liquid assay, such as is described in Enhanced Production, Purification, Characterization and Mechanism of Action of Salivaricin 9 Lantibiotic Produced by Streptococcus salivarius NU10, PLoS One (2013) 8(10): e77751. For viruses, potency can be measured through Selectivity Index (SI) that measures the ratio of the toxic concentration of a sample against its effective bioactive concentration in a cell line experiment. Greater potency would be indicated with a higher SI index compared to a control. Other methods of determining potency of inhibition will be apparent to a skilled worker.

S. salivarius strains useful herein can be characterised at least in part by deferred antagonism assay (P-typing—inhibition of indicator strains), or determining which salivaricins the strain produces. This is described in Tagg and Bannister (1979) J. Med. Microbiol. 12:397. K12 display a P-type 777 (see WO2001027143) and M18 display a P-type 677 on Blood agar with calcium carbonate, and a P-type 777 on Trypticase soy-yeast extract-calcium carbonate agar (see WO2003070919).

The spectrum of inhibitory activity is the number of inhibited microorganisms. In various embodiments, the S. salivarius of the invention inhibits a microorganism or species that is not inhibited by the S. salivarius in the absence of an effective amount of a supplemental saccharide. The applicant has unexpectedly found that supplemental saccharides can not only impact growth and inhibitory activity of S. salivarius probiotics but can also increase the range of microorganisms against which the probiotic is active. This is believed to be the first time that a change in the spectrum of activity for such probiotics has been reported. As described above, inhibitory activity may be measured by determining the size of the zone of inhibition compared to a control sample on an agar plate. A species that is not inhibited will have a zone of inhibition of zero or an SI index of less than 4.9.

In one aspect, the invention provides a composition comprising Streptococcus salivarius and an effective amount of a supplemental saccharide for use in improving the inhibitory profile of the S. salivarius, wherein the S. salivarius is S. salivarius M18, S. salivarius K12, or a combination thereof, and wherein the supplemental saccharide is galactose or raffinose or a combination thereof.

Described herein is a method of inhibiting a non-pathogenic bacteria, the method comprising contacting the bacteria with a composition comprising S. salivarius and an effective amount of a supplemental saccharide, wherein the S. salivarius is S. salivarius M18, S. salivarius K12, or a combination thereof, and wherein the supplemental saccharide is galactose or raffinose or a combination thereof. In various embodiments, the bacteria are selected from L. lactis, S. epidermidis, and S. constellatus.

As discussed in the background, simple sugars are known to promote the growth of bacteria by being consumed as an energy source and produce organic acid by-products, which may have a weak non-selective inhibitory effect on the growth of other bacteria. Surprisingly, the inventors have demonstrated that the improved inhibitory profile of S. salivarius is not solely due to pH effects of the supplementary saccharide (see Examples 4 and 6). Without being bound by theory, the inventors believe the improved inhibitory profile is due to enhanced activity of bacteriocin(s) or other encoded antimicrobial(s) (e.g. non-ribosomal peptide-synthetase (NRPS)). Additionally, the inventors have shown that raffinose unexpectedly influences the morphology of cells resulting in larger sized and more mucoid (sticky mucus-like) colonies making the probiotic more capable of adhering to substrates and thus allowing for enhanced colonisation (see Example 11).

Streptococcus salivarius

S. salivarius is a gram-positive bacterium that predominantly colonises the human oral cavity and are the dominant commensal species. They are highly investigated for use as probiotic bacteria. Two S. salivarius strains have been commercialised by Blis Technologies Ltd with trade names BLIS M18™ and BLIS K12™ for oral and dental health.

A range of S. salivarius strains used in the methods of the invention are known in the art. S. salivarius K12 was deposited with Deutche Sammlung von Mikroorganismen und Zellkulturen GmbH, Mascheroder Weg 1 b, D-38124, Braunschweig, Germany on 8 Oct. 1999, and assigned Accession Nos. DSM 13084. S. salivarius M18 was deposited at Deutsche Sammlung von Mikroorganismen und Zellkulturen GmbH, Mascheroder Weg 1 b, D-38124, Braunschweig, Germany on 12 Dec. 2001, and assigned Accession No. DSM 14685. S. salivarius M18 is described in WO20030709191, incorporated herein by reference.

Bacteriocins are antagonistic substances which have evolved as a conserved biological tool capable of conferring a competitive advantage to the productive organism. Bacteriocins are ribosomally-synthesised antimicrobial peptides produced by bacteria to inhibit the growth of closely related species. A diverse range of bacteriocins have been identified and categorised into four main classes, which are further divided into sub-classes. Bacteriocins classes are determined by parameters such as the productive species, the mechanism action, the spectrum of activity, and the degree of post-translational modification.

BLIS K12™ produces bacteriocins belonging to Class I and Class II: salivaricin A (SEQ ID NOs 15 and 23), salivaricin B (SEQ ID NOs 16 and 24) and salivaricin Q (SEQ ID NOs 17 and 25) (salA, salB and salQ respectively). BLIS M18™ produces bacteriocins belonging to Class I and Class II. salivaricin A (SEQ ID NOs 19 and 27), salivaricin 9 (SEQ ID NOs 20 and 28) and salivaricin Q (SEQ ID NOs 21 and 29) (salA, sal9 and salQ respectively). SalA and SalB are post-translationally modified peptide molecules which display high target specificity for gram-positive, group-A streptococci (i.e. Streptococcus pyogenes). SalA functions by binding to target membrane structures and forming pores in the structure to disrupt the structural integrity of the membrane ultimately leading to cell death. SalB functions by inhibiting target species' enzymatic processes which leads to the prevention of key cellular processes resulting in cell death. A bacteriocin with greater than 90% identity to salQ has also been characterized as a “blpU-like cassette” (Santagati et al., Front. Biosci. (Schol Ed) 2018, 10(2), 238-247.) BLIS K12™ and BLIS M18™ also produce distinct versions of salivaricin MPS which is a Class III bacteriocin. Class III bacteriocins are large, unmodified, heat-labile peptide molecules which target conserved structures expressed by gram-positive streptococci and other bacterial species. BLIS K12™ also produces the antimicrobial NRPS.

The urease protein is important for modulating pH homeostasis for bacteria and when bacteria produce it in the oral cavity it has the effect of modulating the pH of mouth and saliva to less acidic and more towards neutral pH which is of benefit to dental health. Ure C is the alpha subunit of the urease protein encoded by the ureC gene. While the functional protein comprises more subunits, expression of the protein can be inferred based on the ureC gene for BLIS K12™ (SEQ ID NO 18) and BLIS M18™ (SEQ ID NO 22).

In various embodiments, compositions useful in the invention comprise at least 0.1 wt %. of S. salivarius.

In various embodiments, the composition comprises from about 0.1 to about 20 wt % of S. salivarius, for example from about 0.1 to about 18 wt %, about 0.1 to about 16 wt %, about 0.1 to about 15 wt %, about 0.1 to about 14 wt %, about 0.1 to about 12 wt %, about 0.1 to about 10 wt %, about 0.1 to about 9 wt %, about 0.1 to about 8 wt %, about 0.1 to about 7 wt %, about 0.1 to about 6 wt %, about 0.1 to about 5 wt %, about 0.5 to about 18 wt %, about 0.5 to about 16 wt %, about 0.5 to about 15 wt %, about 0.5 to about 14 wt %, about 0.5 to about 12 wt %, about 0.5 to about 10 wt %, about 0.5 to about 9 wt %, about 0.5 to about 8 wt %, about 0.5 to about 7 wt %, about 0.5 to about 6 wt %, about 0.5 to about 5 wt %. about 1 to about 18 wt %, about 1 to about 16 wt %, about 1 to about 15 wt %, about 1 to about 14 wt %, about 1 to about 12 wt %, about 1 to about 10 wt %, about 1 to about 9 wt %, about 1 to about 8 wt %, about 1 to about 7 wt %, about 1 to about 6 wt %, or about 1 to about 5 wt % of S. salivarius.

In various embodiments, the composition comprises the S. salivarius probiotic cells in from about 1×10³ to about 1×10¹³ cfu/g. In various embodiments, the composition comprises S. salivarius cells in an amount of about 1×10⁴ to about 1×10¹², about 1×10⁵ to about 1×10¹², about 1×10⁶ to about 1×10¹², about 1×10⁷ to about 1×10¹² about 1×10⁸ to about 1×10¹², about 1×10⁴ to about 1×10¹⁰, about 1×10⁵ to about 1×10¹⁰, about 1×10⁶ to about 1×10¹⁰, about 1×10⁷ to about 1×10¹⁰, about 1×10⁸ to about 1×10¹⁰, about 1×10⁴ to about 1×10⁹, about 1×10⁵ to about 1×10⁹, about 1×10⁶ to about 1×10⁹, about 1×10⁷ to about 1×10⁹ cfu/g. In various embodiments, the product or composition comprises S. salivarius cells in an amount of about 1×10⁹ cfu/g.

In various embodiments, where multiple strains of S. salivarius are present, the composition comprises at least 0.1 wt % of each strain of S. salivarius.

In various embodiments, where multiple strains of S. salivarius are present, the composition comprises from about 0.1 to about 20 wt % of each strain of S. salivarius, for example from about 0.1 to about 18 wt %, about 0.1 to about 16 wt %, about 0.1 to about 15 wt %, about 0.1 to about 14 wt %, about 0.1 to about 12 wt %, about 0.1 to about 10 wt %, about 0.1 to about 9 wt %, about 0.1 to about 8 wt %, about 0.1 to about 7 wt %, about 0.1 to about 6 wt %, about 0.1 to about 5 wt %, about 0.5 to about 18 wt %, about 0.5 to about 16 wt %, about 0.5 to about 15 wt %, about 0.5 to about 14 wt %, about 0.5 to about 12 wt %, about 0.5 to about 10 wt %, about 0.5 to about 9 wt %, about 0.5 to about 8 wt %, about 0.5 to about 7 wt %, about 0.5 to about 6 wt %, about 0.5 to about 5 wt %, about 1 to about 18 wt %, about 1 to about 16 wt %, about 1 to about 15 wt %, about 1 to about 14 wt %, about 1 to about 12 wt %, about 1 to about 10 wt %, about 1 to about 9 wt %, about 1 to about 8 wt %, about 1 to about 7 wt %, about 1 to about 6 wt %, or about 1 to about 5 wt % of each strain of S. salivarius.

In various embodiments, where multiple strains of S. salivarius are present, the composition comprises from about 1×10³ to about 1×10¹³ cfu/g of each strain of S. salivarius, for example from about 1×10⁴ to about 1×10¹², about 1×10⁵ to about 1×10¹², about 1×10⁶ to about 1×10¹², about 1×10⁷ to about 1×10¹², about 1×10⁸ to about 1×10¹², about 1×10⁴ to about 1×10¹⁰, about 1×10⁵ to about 1×10¹⁰, about 1×10⁶ to about 1×10¹⁰, about 1×10⁷ to about 1×10¹⁰, about 1×10⁸ to about 1×10¹⁰, about 1×10⁴ to about 1×10⁹, about 1×10⁵ to about 1×10⁹, about 1×10⁶ to about 1×10⁹, about 1×10⁷ to about 1×10⁹ cfu/g of each strain of S. salivarius.

### Supplemental Saccharide

Saccharides considered for testing included the monosaccharides glucose, fructose and galactose; the disaccharides sucrose and lactose; and the trisaccharide raffinose.

Lactose is primarily a dairy sugar and is not a preferred ingredient for people on dairy-free diets or people with lactose intolerance. Lactose was therefore excluded from consideration.

As discussed above, sucrose and glucose are cariogenic and generally regarded as having a suppressive effect on antagonistic behavior (see, for example, Reinhold & Titgemeyer, Fritz. (2002). FEMS Microbiology Letters. 209. 141-8; Jankovic & Bruckner, J Mol Microbiol Biotechnol. 2007;12(1-2):114-20). These saccharides were therefore also excluded from consideration.

In various embodiments, the supplemental saccharide is galactose, or raffinose, or a combination thereof. In various embodiments, the supplemental saccharide is galactose. In various embodiments, the supplemental saccharide is raffinose. In various embodiments, the supplemental saccharide is a mixture of galactose and raffinose.

D-Galactose is a monosaccharide sugar. Raffinose is a trisaccharide comprising galactose, glucose, and fructose monomer units.

In various embodiments, the composition comprises less than 20% by weight of each supplemental saccharide. In various embodiments, the composition comprises less than 20% by weight of galactose. In various embodiments, the composition comprises less than 20% by weight of raffinose. In various embodiments, the composition comprises less than 20% by weight of each of galactose and raffinose.

In various embodiments, the composition comprises 0.01-20% by weight of each supplemental saccharide, for example from about 0.01 to about 18%, or about 0.01 to about 15%, or about 0.01 to about 12%, or about 0.01 to about 10%, or about 0.01 to about 9%, or about 0.01 to about 8%, or about 0.01 to about 7%, or about 0.01 to about 6%, or about 0.01 to about 5%, or about 0.01 to about 4%, or about 0.1 to about 20%, or about 0.1 to about 18%, or about 0.1 to about 15%, or about 0.1 to about 12%, or about 0.1 to about 10%, or about 0.1 to about 9%, or about 0.1 to about 8%, or about 0.1 to about 7%, or about 0.1 to about 6%, or about 0.1 to about 5%, or about 0.1 to about 4%, or about 0.25 to about 20%, or about 0.25 to about 18%, or about 0.25 to about 15%, or about 0.25 to about 12%, or about 0.25 to about 10%, or about 0.25 to about 9%, or about 0.25 to about 8%, or about 0.25 to about 7%, or about 0.25 to about 6%, or about 0.25 to about 5%, or about 0.25 to about 4%, or about 0.5 to about 20%, or about 0.5 to about 18%, or about 0.5 to about 15%, or about 0.5 to about 12%, or about 0.5 to about 10%, or about 0.5 to about 9%, or about 0.5 to about 8%, or about 0.5 to about 7%, or about 0.5 to about 6%, or about 0.5 to about 5%, or about 0.5 to about 4%, or about 1 to about 20%, or about 1 to about 18%, or about 1 to about 15%, or about 1 to about 12%, or about 1 to about 10%, or about 1 to about 9%, or about 1 to about 8%, or about 1 to about 7%, or about 1 to about 6%, or about 1 to about 5%, or about 1 to about 4%, or about 1.25 to about 20%, or about 1.25 to about 18%, or about 1.25 to about 15%, or about 1.25 to about 12%, or about 1.25 to about 10%, or about 1.25 to about 9%, or about 1.25 to about 8%, or about 1.25 to about 7%, or about 1.25 to about 6%, or about 1.25 to about 5%, or about 1.25 to about 4%, or about 2 to about 20%, or about 2 to about 18%, or about 2 to about 15%, or about 2 to about 12%, or about 2 to about 10%, or about 2 to about 9%, or about 2 to about 8%, or about 2 to about 7%, or about 2 to about 6%, or about 2 to about 5%, or about 2 to about 4%, or about 2.5 to about 20%, or about 2.5 to about 18%, or about 2.5 to about 15%, or about 2.5 to about 12%, or about 2.5 to about 10%, or about 2.5 to about 9%, or about 2.5 to about 8%, or about 2.5 to about 7%, or about 2.5 to about 6%, or about 2.5 to about 5%, or about 2.5 to about 4% by weight of each supplemental saccharide.

In various embodiments, the composition comprises 0.01-20% by weight of galactose, for example from about 0.01 to about 18%, or about 0.01 to about 15%, or about 0.01 to about 12%, or about 0.01 to about 10%, or about 0.01 to about 9%, or about 0.01 to about 8%, or about 0.01 to about 7%, or about 0.01 to about 6%, or about 0.01 to about 5%, or about 0.01 to about 4%, or about 0.1 to about 20%, or about 0.1 to about 18%, or about 0.1 to about 15%, or about 0.1 to about 12%, or about 0.1 to about 10%, or about 0.1 to about 9%, or about 0.1 to about 8%, or about 0.1 to about 7%, or about 0.1 to about 6%, or about 0.1 to about 5%, or about 0.1 to about 4%, or about 0.25 to about 20%, or about 0.25 to about 18%, or about 0.25 to about 15%, or about 0.25 to about 12%, or about 0.25 to about 10%, or about 0.25 to about 9%, or about 0.25 to about 8%, or about 0.25 to about 7%, or about 0.25 to about 6%, or about 0.25 to about 5%, or about 0.25 to about 4%, or about 0.5 to about 20%, or about 0.5 to about 18%, or about 0.5 to about 15%, or about 0.5 to about 12%, or about 0.5 to about 10%, or about 0.5 to about 9%, or about 0.5 to about 8%, or about 0.5 to about 7%, or about 0.5 to about 6%, or about 0.5 to about 5%, or about 0.5 to about 4%, or about 1 to about 20%, or about 1 to about 18%, or about 1 to about 15%, or about 1 to about 12%, or about 1 to about 10%, or about 1 to about 9%, or about 1 to about 8%, or about 1 to about 7%, or about 1 to about 6%, or about 1 to about 5%, or about 1 to about 4%, or about 1.25 to about 20%, or about 1.25 to about 18%, or about 1.25 to about 15%, or about 1.25 to about 12%, or about 1.25 to about 10%, or about 1.25 to about 9%, or about 1.25 to about 8%, or about 1.25 to about 7%, or about 1.25 to about 6%, or about 1.25 to about 5%, or about 1.25 to about 4%, or about 2 to about 20%, or about 2 to about 18%, or about 2 to about 15%, or about 2 to about 12%, or about 2 to about 10%, or about 2 to about 9%, or about 2 to about 8%, or about 2 to about 7%, or about 2 to about 6%, or about 2 to about 5%, or about 2 to about 4%, or about 2.5 to about 20%, or about 2.5 to about 18%, or about 2.5 to about 15%, or about 2.5 to about 12%, or about 2.5 to about 10%, or about 2.5 to about 9%, or about 2.5 to about 8%, or about 2.5 to about 7%, or about 2.5 to about 6%, or about 2.5 to about 5%, or about 2.5 to about 4% by weight of galactose.

In various embodiments, the composition comprises 0.01-20% by weight of raffinose, for example from about 0.01 to about 18%, or about 0.01 to about 15%, or about 0.01 to about 12%, or about 0.01 to about 10%, or about 0.01 to about 9%, or about 0.01 to about 8%, or about 0.01 to about 7%, or about 0.01 to about 6%, or about 0.01 to about 5%, or about 0.01 to about 4%, or about 0.1 to about 20%, or about 0.1 to about 18%, or about 0.1 to about 15%, or about 0.1 to about 12%, or about 0.1 to about 10%, or about 0.1 to about 9%, or about 0.1 to about 8%, or about 0.1 to about 7%, or about 0.1 to about 6%, or about 0.1 to about 5%, or about 0.1 to about 4%, or about 0.25 to about 20%, or about 0.25 to about 18%, or about 0.25 to about 15%, or about 0.25 to about 12%, or about 0.25 to about 10%, or about 0.25 to about 9%, or about 0.25 to about 8%, or about 0.25 to about 7%, or about 0.25 to about 6%, or about 0.25 to about 5%, or about 0.25 to about 4%, or about 0.5 to about 20%, or about 0.5 to about 18%, or about 0.5 to about 15%, or about 0.5 to about 12%, or about 0.5 to about 10%, or about 0.5 to about 9%, or about 0.5 to about 8%, or about 0.5 to about 7%, or about 0.5 to about 6%, or about 0.5 to about 5%, or about 0.5 to about 4%, or about 1 to about 20%, or about 1 to about 18%, or about 1 to about 15%, or about 1 to about 12%, or about 1 to about 10%, or about 1 to about 9%, or about 1 to about 8%, or about 1 to about 7%, or about 1 to about 6%, or about 1 to about 5%, or about 1 to about 4%, or about 1.25 to about 20%, or about 1.25 to about 18%, or about 1.25 to about 15%, or about 1.25 to about 12%, or about 1.25 to about 10%, or about 1.25 to about 9%, or about 1.25 to about 8%, or about 1.25 to about 7%, or about 1.25 to about 6%, or about 1.25 to about 5%, or about 1.25 to about 4%, or about 2 to about 20%, or about 2 to about 18%, or about 2 to about 15%, or about 2 to about 12%, or about 2 to about 10%, or about 2 to about 9%, or about 2 to about 8%, or about 2 to about 7%, or about 2 to about 6%, or about 2 to about 5%, or about 2 to about 4%, or about 2.5 to about 20%, or about 2.5 to about 18%, or about 2.5 to about 15%, or about 2.5 to about 12%, or about 2.5 to about 10%, or about 2.5 to about 9%, or about 2.5 to about 8%, or about 2.5 to about 7%, or about 2.5 to about 6%, or about 2.5 to about 5%, or about 2.5 to about 4% by weight of raffinose.

In various embodiments, where a mixture of galactose and raffinose is present, the composition comprises 0.01-20% by weight of each of galactose and raffinose, for example from about 0.01 to about 18%, or about 0.01 to about 15%, or about 0.01 to about 12%, or about 0.01 to about 10%, or about 0.01 to about 9%, or about 0.01 to about 8%, or about 0.01 to about 7%, or about 0.01 to about 6%, or about 0.01 to about 5%, or about 0.01 to about 4%, or about 0.1 to about 20%, or about 0.1 to about 18%, or about 0.1 to about 15%, or about 0.1 to about 12%, or about 0.1 to about 10%, or about 0.1 to about 9%, or about 0.1 to about 8%, or about 0.1 to about 7%, or about 0.1 to about 6%, or about 0.1 to about 5%, or about 0.1 to about 4%, or about 0.25 to about 20%, or about 0.25 to about 18%, or about 0.25 to about 15%, or about 0.25 to about 12%, or about 0.25 to about 10%, or about 0.25 to about 9%, or about 0.25 to about 8%, or about 0.25 to about 7%, or about 0.25 to about 6%, or about 0.25 to about 5%, or about 0.25 to about 4%, or about 0.5 to about 20%, or about 0.5 to about 18%, or about 0.5 to about 15%, or about 0.5 to about 12%, or about 0.5 to about 10%, or about 0.5 to about 9%, or about 0.5 to about 8%, or about 0.5 to about 7%, or about 0.5 to about 6%, or about 0.5 to about 5%, or about 0.5 to about 4%, or about 1 to about 20%, or about 1 to about 18%, or about 1 to about 15%, or about 1 to about 12%, or about 1 to about 10%, or about 1 to about 9%, or about 1 to about 8%, or about 1 to about 7%, or about 1 to about 6%, or about 1 to about 5%, or about 1 to about 4%, or about 1.25 to about 20%, or about 1.25 to about 18%, or about 1.25 to about 15%, or about 1.25 to about 12%, or about 1.25 to about 10%, or about 1.25 to about 9%, or about 1.25 to about 8%, or about 1.25 to about 7%, or about 1.25 to about 6%, or about 1.25 to about 5%, or about 1.25 to about 4%, or about 2 to about 20%, or about 2 to about 18%, or about 2 to about 15%, or about 2 to about 12%, or about 2 to about 10%, or about 2 to about 9%, or about 2 to about 8%, or about 2 to about 7%, or about 2 to about 6%, or about 2 to about 5%, or about 2 to about 4%, or about 2.5 to about 20%, or about 2.5 to about 18%, or about 2.5 to about 15%, or about 2.5 to about 12%, or about 2.5 to about 10%, or about 2.5 to about 9%, or about 2.5 to about 8%, or about 2.5 to about 7%, or about 2.5 to about 6%, or about 2.5 to about 5%, or about 2.5 to about 4% of each of galactose and raffinose.

### Upregulated Genes

In one aspect, the invention provides a method for upregulating one or more genes in Streptococcus salivarius, comprising formulating the S. salivarius in a composition comprising an effective amount of a supplemental saccharide,
wherein the Streptococcus salivarius is Streptococcus salivarius M18, Streptococcus salivarius K12, or a combination thereof, and
wherein the supplemental saccharide is galactose, or raffinose, or a combination thereof.

In some embodiments, the upregulated gene(s) may comprise or consist of a polynucleotide sequence that encodes a lantibiotic peptide or a bacteriocin, such as a Class I or Class II lantibiotic peptide or bacteiocin. For example, as described above, BLIS K12™ produces the bacteriocins salivaricin A, salivaricin B, and salivaricin Q, encoded by salA, salB and salQ respectively (SEQ ID NOs 15, 16, and 17 respectively). The peptide sequences of these bacteriocins are presented in SEQ ID NOs 23, 24, and 25, respectively.

As a further example, as described above, BLIS M18™ produces the bacteriocins salivaricin A, salivaricin 9, and salivaricin Q, encoded by salA, sal9 and salQ respectively (SEQ ID NOs 19, 20, and 21 respectively). The peptide sequences of these bacteriocins are presented in SEQ ID NOs 27, 28, and 29 respectively.

In some embodiments, the upregulated gene(s) may comprise or consist of a polynucleotide sequence that encodes a subunit of a urease protein. In some embodiments, the upregulated gene is ureC, for example, the BLIS K12™ ureC (SEQ ID NO 18) or the BLIS M18™ ureC (SEQ ID NO 22).

In some embodiments, at least one of the upregulated gene(s) comprises or consists of a polynucleotide sequence with at least 70% sequence identity to any one of SEQ ID NOs 15-22, or at least one of the upregulated gene(s) comprises or consists of a polynucleotide sequence that encodes a polypeptide with at least 70% sequence identity to any one of SEQ ID NOs 23-30.

In some embodiments, at least one of the upregulated gene(s) is a salA gene, a salB gene, a salQ gene, a sal9 gene, and/or a ureC gene, or a variant of any of these.

These genes and variants thereof can be readily identified by techniques known in the art. For example, these genes may be identified by sequence similarity to known salA, salB, salQ, sal9, or ureC genes, or to genes encoding salA, salB, salQ, sal9, or ureC proteins, using sequence alignment tools. Alternatively, genes encoding proteins may be identified by structural similarity to known salA, salB, salQ, sal9, or ureC proteins using structural alignment.

For example, these genes may be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, MD 20894 USA. The use of the BLAST family of algorithms, including BLASTN, BLASTP, and BLASTX, is described in the publication of Altschul et al., Nucleic Acids Res. 25: 3389-3402, 1997.

Multiple sequence alignments of a group of related sequences can be carried out with CLUSTALW (Thompson, J.D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680) CLUSTAL Omega (Sievers et al., (2011). Molecular Systems Biology 7:539, https://www.ebi.ac.uk/Tools/msa/clustalo/) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

Another example of a protein domain model database is Pfam (Sonnhammer et al., 1997, A comprehensive database of protein families based on seed alignments, Proteins, 28: 405-420; Finn et al., 2010, The Pfam protein families database', Nucl. Acids Res., 38: D211-D222). “Pfam” refers to a large collection of protein domains and protein families maintained by the Pfam Consortium and available at several sponsored world wide web sites, including: pfam.xfam.org/ (European Bioinformatics Institute (EMBL-EBI). The latest release of Pfam is Pfam 35.0 (November 2021). Pfam domains and families are identified using multiple sequence alignments and hidden Markov models (HMMs). Pfam-A family or domain assignments, are high quality assignments generated by a curated seed alignment using representative members of a protein family and profile hidden Markov models based on the seed alignment. (Unless otherwise specified, matches of a queried protein to a Pfam domain or family are Pfam-A matches.) All identified sequences belonging to the family are then used to automatically generate a full alignment for the family (Sonnhammer (1998) Nucleic Acids Research 26, 320-322; Bateman (2000) Nucleic Acids Research 26, 263-266; Bateman (2004) Nucleic Acids Research 32, Database Issue, D138-D141; Finn (2006) Nucleic Acids Research Database Issue 34, D247-251; Finn (2010) Nucleic Acids Research Database Issue 38, D21 1-222). By accessing the Pfam database, for example, using the above-referenced website, protein sequences can be queried against the HMMs using HMMER homology search software {e.g., HMMER2, HMMER3, or a higher version, hmmer.org). Significant matches that identify a queried protein as being in a pfam family (or as having a particular Pfam domain) are those in which the bit score is greater than or equal to the gathering threshold for the Pfam domain. Expectation values (e values) can also be used as a criterion for inclusion of a queried protein in a Pfam or for determining whether a queried protein has a particular Pfam domain, where low e values (much less than 1.0, for example less than 0.1, or less than or equal to 0.01) represent low probabilities that a match is due to chance.

Some specific examples of known genes from S. salivarius are provided herein according to the following table.

| Strain | Gene name | DNA sequence | Protein sequence |
|---|---|---|---|
| K12 | SalA | SEQ ID NO 15 | SEQ ID NO 23 |
| K12 | SalB | SEQ ID NO 16 | SEQ ID NO 24 |
| K12 | SalQ | SEQ ID NO 17 | SEQ ID NO 25 |
| K12 | UreC | SEQ ID NO 18 | SEQ ID NO 26 |
| M18 | SalA | SEQ ID NO 19 | SEQ ID NO 27 |
| M18 | Sal9 | SEQ ID NO 20 | SEQ ID NO 28 |
| M18 | SalQ | SEQ ID NO 21 | SEQ ID NO 29 |
| M18 | UreC | SEQ ID NO 22 | SEQ ID NO 30 |

In some embodiments, at least one of the upregulated gene(s) comprises or consists of a polynucleotide sequence with at least 70% identity to any one of SEQ ID Nos 15-22, preferably at least 71%, 72%, 73%, 74%, 75%, 76%, 77% 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

In some embodiments, at least one of the upregulated gene(s) comprises or consists of a polynucleotide sequence that encodes a polypeptide with at least 70% identity to any one of SEQ ID Nos 23-30, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

In some embodiments, at least one of the upregulated gene(s) is a salA gene that comprises or consists of a polynucleotide sequence with at least 70% identity to SEQ ID NO 15 or 19, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity; or that encodes a polypeptide with at least 70% identity to SEQ ID NO 23 or 27, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

In some embodiments, at least one of the upregulated gene(s) is a salB gene that comprises or consists of a polynucleotide sequence with at least 70% identity to SEQ ID NO 16, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity; or that encodes a polypeptide with at least 70% identity to SEQ ID NO 24, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

In some embodiments, at least one of the upregulated gene(s) is a salQ gene that comprises or consists of a polynucleotide sequence with at least 70% identity to SEQ ID NO 17 or 21, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity; or that encodes a polypeptide with at least 70% identity to SEQ ID NO 25 or 29, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

In some embodiments, at least one of the upregulated gene(s) is a sal9 gene that comprises or consists of a polynucleotide sequence with at least 70% identity to SEQ ID NO 20, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity; or that encodes a polypeptide with at least 70% identity to SEQ ID NO 28, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

In some embodiments, at least one of the upregulated gene(s) is a ureC gene that comprises or consists of a polynucleotide sequence with at least 70% identity to SEQ ID NO 18 or 22, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity; or that encodes a polypeptide with at least 70% identity to SEQ ID NO 26 or 30, preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity.

### Determining Polynucleotide Sequence Identity

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:

bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences online at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Another method for calculating polynucleotide % sequence identity is based on aligning sequences to be compared using Clustal X (Jeanmougin et al., 1998, Trends Biochem. Sci. 23, 403-5.)

Variant polynucleotides or genes as disclosed herein also encompass polynucleotides or genes that differ from the sequences as herein disclosed but that, as a consequence of the degeneracy of the genetic code, encode a polypeptide having similar activity to a polypeptide encoded by a polynucleotide disclosed herein. A sequence alteration that does not change the amino acid sequence of the polypeptide is a “silent variation”.

Variant polynucleotides or genes as disclosed herein may comprise sequence alterations resulting in conservative substitutions of one or several amino acids in the encoded polypeptide sequence without significantly altering its biological activity.

Variant polynucleotides due to silent variations and conservative substitutions in the encoded polypeptide sequence may be determined using the publicly available bl2seq program from the BLAST suite of programs (version 2.2.5 [November 2002]) from NCBI (ftp://ftp.ncbi.nih.gov/blast/) via the tblastx algorithm as previously described.

### Determining Polypeptide Sequence Identity

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Another method for calculating polypeptide % sequence identity is based on aligning sequences to be compared using Clustal X (Jeanmougin et al., 1998, Trends Biochem. Sci. 23, 403-5.)

Polypeptide variants as disclosed herein also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/). The similarity of polypeptide sequences may be examined using the following unix command line parameters:

bl2seq -i peptideseq1 -j peptideseq2 -F F -p blastp

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Variant polypeptide sequences preferably exhibit an E value of less than 1×10⁻¹ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰ and most preferably less than 1×10⁻¹⁰⁰ when compared with any one of the specifically identified sequences.

Variant polypeptide sequences may comprise conservative substitutions of one or several amino acids of a described polypeptide sequence without significantly altering its biological activity.

Identity is found over a comparison window of at least 20 amino acid positions, preferably at least 50 amino acid positions, more preferably at least 100 amino acid positions, and most preferably over the entire length of a polypeptide as herein disclosed.

### Inhibited Bacteria

In various embodiments, the method increases the inhibitory profile of S. salivarius against skin, oral, dental, mucosal (e.g. oral, rectal, vaginal) and/or ENTR microorganisms including pathogenic and other non-pathogenic microorganisms.

Microorganisms may be non-pathogenic. Subjects have an existing microflora which is generally not harmful, or may be beneficial to the subject. Examples of such non-pathogenic microflora bacteria include S. salivarius spp., for example, S. epidermidis spp., L. lactis spp., and S. constellatus spp.

In some embodiments it may be useful to inhibit or reduce the population of such non-pathogenic microorganisms. For example, to facilitate colonisation with a Blis-producing S. salivarius such as K12 or M18.

In various embodiments, the skin, oral, dental, mucosal, and/or ENT microorganism is selected from S. aureus spp., S. intermedius spp., S. saprophyticus spp., M. catarrhalis spp., H. influenzae spp., S. pyogenes spp., P. aeruginosa spp., S. mutans spp., S. pneumoniae spp., C. acnes spp., C. albicans spp. S. sobrinus spp., Corynebacterium spp., F. nucleatum spp., A. actinomycetemcomitans spp., P. gingivalis spp., Tannerella forsythia spp., Treponema denticola spp., P. intermedia spp., Prevotella spp., A. viscosus spp., S. equismillis spp., S. dygalactiae spp., S. sanguis spp., S. cohnii spp., B. intermedius spp., A. parvulum spp., E. saburreum spp., E. sulci spp., P. micra spp., S. moorei spp., S. agalactiae spp., C. minutissimus spp., P. propionicus spp., S. agalactiae spp., S. dysgalactiae spp., S. simulans spp., S. xylosus spp., Tinea pedis infection causing fungi, S. salivarius spp. Other than K12 or M18, L. lactis spp., S. epidermidis spp., S. constellatus spp. or any combination of any two or more thereof.

In various embodiments, the oral and/or dental microorganism is selected from S. intermedius spp., M. catarrhalis spp., H. influenzae spp., S. pyogenes spp., S. mutans spp., S. pneumoniae spp., F. nucleatum spp., A. actinomycetemcomitans spp., P. intermedia spp., Prevotella spp., A. viscosus spp., S. sobrinus spp., B. intermedius spp., A. parvulum spp., E. saburreum spp., E. sulci spp., P. micra spp., S. moorei spp., S. agalactiae spp., or any combination of any two or more thereof.

In various embodiments, the skin microorganism is selected from S. aureus spp., S. saprophyticus spp., S. pyogenes spp., C. acnes spp., C. albicans spp. S. cohnii spp., C. minutissimus spp., P. propionicus spp., S. agalactiae spp., S. dysgalactiae spp., S. simulans spp., S. xylosus spp., Tinea pedis infection causing fungi, S. epidermidis spp., or any combination of any two or more thereof.

In various embodiments, the skin, oral, dental, mucosal, and/or ENTR microorganism is selected from S. aureus spp., S. saprophyticus spp., M. catarrhalis spp., H. influenzae spp., S. pyogenes spp., P. aeruginosa spp., S. mutans spp., S. pneumoniae spp., S. salivarius spp. Other than K12 or M18, L. lactis spp., S. epidermidis spp., S. constellatus spp., or any combination of any two or more thereof.

In various embodiments, the skin, oral, dental, mucosal, and/or ENTR microorganism is selected from S. aureus A222, S. aureus 20, S. aureus 14, S. aureus 19, S. aureus A504, S. aureus ATCC 6538, S. saprophyticus ATCC 15305, M. catarrhalis TW1, M. catarrhalis TW2, H. influenzae TW5, S. pyogenes M76, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, P. aeruginosa 12, P. aeruginosa ATCC 27853, S. mutans OMZ175, S. mutans FW75, S. pneumoniae D39, S. pneumoniae RX1, S. pneumoniae PK8, S. equismillis Bris 2, S. dygalactiae T277, C. acnes ATC 6919, S. sanguis K11, S. sobrinus OMZ176, S. cohnii, S. simulans, L. lactis T-21, S. epidermidis 11, S. epidermidis E30, S. constellatus T-29, S. salivarius 6, S. salivarius 193, S. salivarius 20P3, or any combination of any two or more thereof.

In various embodiments, the skin, oral, dental, mucosal, and/or ENTR microorganism is selected from S. aureus A222, S. aureus 20, S. aureus 14, S. aureus 19, S. aureus A504, S. saprophyticus ATCC 15305, M. catarrhalis TW1, M. catarrhalis TW2, H. influenzae TW5, S. pyogenes M76, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, P. aeruginosa 12, S. mutans OMZ175, S. pneumoniae D39, L. lactis T-21, S. epidermidis 11, S. constellatus T-29, S. salivarius 6, S. salivarius 193, S. salivarius 20P3, or any combination of any two or more thereof.

S. salivarius strains having anti-viral activity, particularly anti-SARS-CoV2 (Covid-19) activity, are described in PCT/NZ2021/050054 (Blis Technologies Ltd). Also described are prophylactic and therapeutic compositions and methods for treating respiratory viruses. The data shows that S. salivarius K12, M18 have varying anti-viral activity against SARS-CoV2, influenza A, influenza B, and respiratory syncytial virus (RSV). Accordingly, the microorganism to be inhibited herein also includes respiratory viruses such as SARS-CoV2, Influenza A, Influenza B, and Respiratory Syncytial Virus (RSV).

In various embodiments, the skin, oral, dental, mucosal, and/or ENT microorganism is a Streptococcus or Staphylococcus bacteria. In various embodiments, the Streptococcus or Staphylococcus bacteria is selected from S. aureus spp., S. saprophyticus spp., S. mutans spp., S. pyogenes spp., and S. pneumoniae spp., S. constellatus spp., and S. salivarius spp. In various embodiments, the Staphylococcus bacteria is selected from S. aureus A222, S. saprophyticus ATCC15305, and the Streptococcus bacteria is selected from S. mutans OMZ175, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, S. pneumoniae D39, S. salivarius spp., e.g. S. salivarius 6, S. salivarius 193, S. salivarius 20P3, and S. constellatus T-29.

In various embodiments, the Streptococcus or Staphylococcus bacteria is selected from S. aureus spp., S. saprophyticus spp., S. mutans spp., S. salivarius spp., S. constellatus spp., S. pyogenes spp., and S. pneumoniae spp., S. equisimilis spp., S. dysgalactiae spp., and the S. salivarius is K12. In various embodiments, Staphylococcus bacteria is selected from S. aureus A222, S. saprophyticus ATCC15305, and the Streptococcus bacteria is selected from S. mutans OMZ175, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, S. pneumoniae D39, S. equisimilis Bris 2, S. dysgalactiae T277, S. salivarius spp., e.g. S. salivarius 6, S. salivarius 193, S. salivarius 20P3, and S. constellatus T-29; and the S. salivarius strain is K12.

In various embodiments, the skin, oral, dental, mucosal and/or ENTR microorganism is selected from S. aureus spp., S. saprophyticus spp., L. lactis spp., S. epidermidis spp., S. salivarius spp., M. catarrhalis spp., S. mutans spp., H. influenzae spp., S. pneumoniae spp., S. pyogenes spp., L. lactis T-21, S. epidermidis 11, S. epidermidis E30, and S. constellatus T-29; the S. salivarius strain is K12; and the supplemental saccharide is galactose.

In various embodiments, the skin, oral, dental, mucosal and/or ENTR microorganism is selected from S. aureus A222, S. aureus 20, S. aureus 14, S. aureus 19, S. aureus A504, S. saprophyticus ATCC 15305, L. lactis T-21, S. epidermidis 11, M. catarrhalis TW1, M. catarrhalis TW2, S. mutans OMZ175, H. influenzae, S. pneumoniae D39, S. pyogenes 71-698, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, L. lactis T-21, S. epidermidis 11, S. epidermidis E30, S. constellatus T-29; the S. salivarius strain is K12; and the supplemental saccharide is galactose.

In various embodiments, the skin, oral, dental, mucosal, and/or ENTR microorganism is selected from S. aureus spp., S. saprophyticus spp., L. lactis spp., and S. epidermidis spp.; the S. salivarius strain is K12; and the supplemental saccharide is raffinose.

In various embodiments, the skin, oral, dental, mucosal and/or ENTR microorganism is selected from S. aureus A222, S. aureus 20, S. aureus 14, S. aureus 19, S. aureus A504, S. saprophyticus ATCC 15305, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, L. lactis T-21, S. epidermidis 11, S. salivarius 6, S. salivarius 193, and S. salivarius 20P3; the S. salivarius strain is K12; and the supplemental saccharide is raffinose.

In various embodiments, the skin, oral, dental, mucosal, and/or ENTR microorganism is selected from S. constellatus spp., S. pyogenes spp., S. pneumoniae spp., S. salivarius spp., S. mutans spp., S. saprophyticus spp., S. aureus spp., M. catarrhalis spp., L. lactis spp., H. influenzae spp., and P. aeruginosa spp.; the S. salivarius strain is M18; and the supplemental saccharide is raffinose.

In various embodiments, the skin, oral, dental, mucosal, and/or ENTR microorganism is selected from, S. pyogenes M76, S. pneumoniae D39, S. mutans OMZ175, S. saprophyticus ATCC 15305, S. aureus A222, M. catarrhalis TW1, M. catarrhalis TW2, L. lactis T-21, H. influenzae TW5, P. aeruginosa 12, S. pyogenes 71-698, S. pyogenes FF22, S. pyogenes 71-679, S. pyogenes W-1, S. pyogenes M57, S. pyogenes EMM92, S. pyogenes M66, S. pyogenes M74, S. constellatus T-29, S. salivarius 6, S. salivarius 193, and S. salivarius 20P3; the S. salivarius strain is M18; and the supplemental saccharide is raffinose.

In various embodiments, the bacteria are selected from S. pyogenes spp., and S. pneumoniae spp.; and the S. salivarius strain is M18. In various embodiments, the bacteria is selected from S. pyogenes 71-698, and S. pneumoniae D39; and the S. salivarius strain is M18.

In various embodiments the microorganism is a virus selected from SARS-CoV2, Influenza A, Influenza B, and RSV.

### Prevention or Treatment of Diseases or Inhibition of Microorganisms

In one aspect, the invention provides a method of treating or preventing a disease or disorder comprising administering to subject in need thereof a composition comprising S. salivarius and an effective amount of a supplemental saccharide; wherein the S. salivarius is S. salivarius M18, S. salivarius K12, or a combination thereof; and wherein the supplemental saccharide is galactose or raffinose or a combination thereof.

In one aspect, the invention relates to use of S. salivarius and a supplemental saccharide in the manufacture of a medicament for the treatment or prevention of a disease or disorder; wherein the S. salivarius is Streptococcus salivarius M18, S. salivarius K12, or a combination thereof; and wherein the supplemental saccharide is galactose or raffinose or a combination thereof.

In one aspect, the invention provides a composition comprising S. salivarius and an effective amount of a supplemental saccharide for use in the treatment or prevention of a disease or disorder; wherein the S. salivarius is S. salivarius M18, S. salivarius K12, or a combination thereof, and wherein the supplemental saccharide is galactose or raffinose or a combination thereof.

In one aspect, the invention provides a composition comprising S. salivarius and an effective amount of a supplemental saccharide; wherein the S. salivarius is S. salivarius M18, S. salivarius K12, or a combination thereof, and wherein the supplemental saccharide is galactose or raffinose or a combination thereof.

In various embodiments, the composition or therapeutic formulation improves the inhibitory profile, and/or improves the mucoid properties of the S. salivarius.

In various embodiments, the disease or disorder is caused by a skin, oral, dental, mucosal, or ENTR pathogen.

In various embodiments, the disease or disorder is otitis media, sore throat, tooth decay, acute pharyngitis, tonsillitis, pneumonia, COPD, periodontal disease, gingivitis, halitosis, dental caries, sepsis, meningitis, vaginitis, body odour, acne, actinomycosis, psoriasis, erythrasma, cellulitis, impetigo, atopic dermatitis, bacteraemia, soft tissue infections, erythema, nosocomial, erythema, SARS-CoV2, Influenza A, Influenza B, and RSV, candidiasis (oral thrush), athlete's foot. Or any combination of any two or more thereof.

In various embodiments, the disease or disorder is caused by pathogenic bacteria. In various embodiments, the disease or disorder is caused by pathogenic Streptococcus bacteria.

In various embodiments, the disease or disorder is otitis media, sore throat, tooth decay, acute pharyngitis, tonsillitis, pneumonia, COPD, periodontal disease, gingivitis, halitosis, dental caries, sepsis, meningitis, vaginitis, body odour, acne, actinomycosis, psoriasis, erythrasma, cellulitis, impetigo, atopic dermatitis, bacteraemia, soft tissue infections, erythema, nosocomial, erythema, or any combination of any two or more thereof.

In various embodiments, the disease or disorder is caused by a pathogenic virus.

In various embodiments, the disease or disorder is SARS-CoV2, Influenza A, Influenza B, or RSV.

In various embodiments, the disease or disorder is caused by a pathogenic fungus (e.g. yeast or skin mycoses).

In various embodiments, the disease or disorder is candidiasis (oral thrush), athlete's foot (Tinea pedis), or other Tinea infections.

In various embodiments, the subject is a mammal, including humans, dogs, cats, horses, sheep, cows and other domestic and farm animals. In various embodiments, the subject is a non-human subject. In various embodiments, the subject is a human. In various embodiments, the subject is an infant, child or adult.

In one aspect the invention also relates to a method of inhibiting a microorganism sensitive to Blis-producing S. salivarius, comprising administering to subject in need thereof a composition of the invention, or a therapeutic formulation of the invention. The microorganism to be inhibited may be a non-pathogenic microorganism for example S. salivarius spp., S. epidermidis spp., L. lactis spp. or S. constellatus spp. To facilitate adhesion and colonisation by S. salivarius used in the invention, it may be desirable to reduce populations of such other non-pathogenic microorganisms.

### Raw Ingredient Composition

Lyoprotectants and cryoprotectants are commonly used in the manufacture of products containing BLIS-producing strains (including S. salivarius containing products) to protect and maintain cell viability. Lyoprotectants protect during drying, while cryoprotectants protect during freezing. The same composition can have both functions, and unless otherwise specified, the terms are used interchangeably herein. Suitable lyoprotectants or cryoprotectants will be known to a person skilled in the art.

In various embodiments, the lyoprotectant may be selected from sodium caseinate, peptone, skim milk powder, whey protein, trehalose, glycerol, betaine, sucrose, galactose, glucose, lactose, lactitol, mannitol, maltodextrin, sodium citrate, and combinations thereof.

In various embodiments, the lyoprotectant may be a mixture of trehalose, lactitol, and maltodextrin.

In various embodiments, the composition is dairy-free. In various embodiments, the composition does not comprise any dairy-derived ingredients. In various embodiments, the lyoprotectant may be a mixture of sucrose, sodium citrate, and maltodextrin or trehalose.

In various embodiments, the composition is a powder, for example a powder which has been prepared by admixing a powder of freeze-dried S. salivarius with a powder of the supplemental saccharide, or by co-freeze-drying S. salivarius with supplemental saccharide. The inventors have found that the supplemental saccharides investigated herein do not affect the stability of freeze-dried raw ingredient powders of S. salivarius (data unreported).

A skilled worker would appreciate the composition may comprise other excipients including a diluent or a flow aid.

Use of Raw Ingredient in Therapeutic Formulation e.g. Lozenge Etc.

The composition may be formulated into therapeutic formulations for administration by various methods. A “therapeutic formulation” is a composition appropriate for use in prophylactic or therapeutic treatment of an individual in need of same. In general, therapeutic formulations are composed of a S. salivarius strain and supplemental saccharide discussed above and a pharmaceutically acceptable carrier, diluent and/or excipient.

In one aspect, the composition of the invention is formulated into a therapeutic formulation. In various embodiments, the composition or therapeutic formulation is formulated for oral, dental, nasal, ENTR, or topical administration.

In various embodiments, the therapeutic formulation is a powder, lozenge, nasal spray, nasal gel, nasal drop, oral drop, oral gel, oral spray, inhalable, aerosol, topical composition, chewable, melt, film, gummy, toothpaste, tooth-gel, varnish, mousse, mouthwash, food product (e.g. yoghurt), cream, gel, spray, deodorant, serum, lotion, balm, moisturiser, pessary or suppository Slow or sustained release products which maintain the level of supplemental saccharide in the oral cavity or ENTR are preferred in some embodiments.

Slow or sustained release products which maintain the level of supplemental saccharide in the oral cavity or ENTR are preferred in some embodiments. Such slow- or sustained release products are known in the art and include multilayer tablets, slow or fast dissolving melts, films, chewing gum, gels, mucoadhesive or buccal adhesive delivery systems.

An “acceptable carrier, diluent and/or excipient” means a vehicle for delivery of a S. salivarius strain or extract, to a surface or a host, in which the vehicle is compatible with bacterial cell viability, or activity of the extract. Acceptable carriers, diluents and excipients suitable for use in the administration of viable streptococcal strains, particularly S. salivarius strains and extracts are well known to those skilled in the art (see, for example, Remington's Pharmaceutical Sciences, 22^{nd} ed., Gennaro, ed., 2013, Mack Publishing Co., Easton, Pa.), incorporated herein by reference. Suitable carriers are generally inert and can be either solid or liquid.

In various embodiments, the carrier is a pharmaceutically acceptable carrier. Such pharmaceutically acceptable carriers may be aqueous or non-aqueous solutions, suspensions, and emulsions. A variety of pharmaceutically acceptable carriers suitable for administration of viable or lyophilized bacteria are well known in the art (See for example Remington's supra.; and the pharmaceutical composition LACTINEXä (Hynson, Westcott and Dunning, Baltimore, Md. USA), a commercially available formulation for oral administration of viable lactobacilli). Suitable solid carriers known in the art include, for example, magnesium carbonate; magnesium stearate; celluloses; talc; sugars such as maltose, fructose, sucrose, mannitol, lactose, isomalt, maltodextrin, starches; flours; oligosaccharides and skim milk, and similar edible powders, but are not limited thereto.

Typical diluents, by way of example are: starches; lactose; mannitol; kaolin; calcium phosphate or sulphate; inorganic salts such as sodium chloride; and powdered sugars or celluloses.

The therapeutic formulation may also include excipients such as resins; fillers; binders; lubricants; solvents; glidants; disintegrants; preservatives; buffers; flavourings; colourings; sweeteners; and fragrances as appropriate.

Typical binders include starch; gelatin; sugars such as lactose, fructose, and glucose; and the like. Natural and synthetic gums are also convenient, including acacia; alginates; locust bean gum; methylcellulose; polyvinylpyrrolidine; tragacanth gum; Xanthan gum; and the like. Polyethylene glycol; ethyl cellulose; and waxes can also serve as binders.

Lubricants to prevent sticking to a die during manufacture include slippery solids such as talc, silica, magnesium and calcium stearate, polyethylene glycol, stearic acid and hydrogenated vegetable oils.

Disintegrators are substances which swell when wetted to break up the composition and release the streptococci or extract. The disintegrators include starches; clays; celluloses; algins and gums; more particularly corn and potato starches; methylcellulose; agar; bentonite; wood cellulose; cation exchange resins; alginic acid; guar gum; citrus pulp; carboxymethylcellulose; powdered sponge; and sodium lauryl sulfate.

For delivery to the respiratory tract, the composition can also be in a form for administration by inhalation. The inhaled product is typically in powdered or micronized powder form, or liquid form. The product can conveniently be administered using an inhaler, nebuliser, atomiser, or any other recognised device for delivery to the respiratory tract. Carriers for inhalable products are well known in the art and include lactose, erythritol, sorbitol, and cyclodextrin.

The therapeutic formulation can additionally contain nutrients to maintain the viability and enhance the efficacy of the bacterium in the formulation. Further ingredients useful in a composition are agents that selectively enhance growth of desirable bacteria over non desirable organisms.

In various embodiments, the therapeutic formulation comprises a buffering agent (phosphate buffers, citric acid), calcium carbonate, multivitamin, mineral (e.g. Zinc, Vitamin C and D), antioxidant (berries e.g. blackcurrant, quercetin, liquorice), fluoride, xylitol, yeast extract (Saccharomyces cerevisiae or Saccharomyces boulardii), lysate, extract (Bifidobacterium, Lactobacillus), and/or yogurt culture.

In various embodiments, the therapeutic formulation further comprises other potentiating agents to promote the production or activity of a composition. In various embodiments, the potentiating agents are selected from carbohydrates, for example, oligosaccharides such as Nutriose® FB (Roquette Freres, Lestrem, France), maltodextrose, and lactulose; prebiotic agents; chemicals such as reducing agents, for example cysteine and mercaptoethanol; and metal ions such as magnesium.

Therapeutic formulations can also be formulated to contain flavouring agents, colouring agents, sweeteners (xylitol, maltodextrin, monk fruit extracts, stevia, aspartame), taste-masking agents (Smoothenol®), fragrances, or other compounds which increase the appeal of the product to a patient and/or enhance patient compliance without compromising the effectiveness of the product. Methods for preparation of therapeutic formulations for inhalable administration are well known in the art (see, for example, Remington's Pharmaceutical Sciences, 22nd ed., supra, incorporated herein by reference).

A topical therapeutic formulation may comprise other additives conventionally used in a topical composition, such as a moisturiser. Art skilled readers will further appreciate that additives need to be compatible with probiotic viability and efficacy. Such additives may provide or improve a therapeutic, cosmetic, stability, and/or appearance property of the therapeutic formulation. Examples of suitable additives include, but are not limited to, a carrier (e.g. vegetable oils, triglycerides, glycerol, propylene glycol, water, saline), a surfactant, a dispersant, an emulsifier, an inhibitory activity enhancer, a buffering agent, an antibacterial agent, a prebiotic, a fragrance, an antioxidant, a colourant, a skin protective agent, an anti-aging agent (hyaluronic acid, ceramides, olive squalene), an antimicrobial, an aluminium salt, a mineral pigment, an odour absorbant or neutraliser, or a sunscreen agent. Such additives may be included in the therapeutic formulation of the invention in amounts typical for topical formulations. A variety of pharmaceutically acceptable additives suitable for topical application of viable or lyophilized bacteria are well known in the art.

It may be advantageous to formulate the composition into a slow or sustained release composition or therapeutic formulation. In various embodiments, the composition is formulated in a slow-release composition. In various embodiments, the composition is formulated in a two-part composition that provides immediate release of the S. salivarius and the supplemental saccharide, and a slow release of further supplemental saccharide.

### Methods of Administration

The reader will appreciate that the compositions and formulations of the invention may be administered according to a wide range of protocols to inhibit microbial populations for both therapeutic and non-therapeutic purposes. Any protocols known in the art for administration of S. salivarius K12 and M18 may be used.

In various embodiments, the therapeutic formulation is administered orally once, twice, three, four times, or up to twelve times daily. In various embodiments, the therapeutic formulation is administered orally via a lozenge, powder, melt, mouthwash, or toothpaste. For the treatment of halitosis, it is recommended to pre-treat with a mouthwash such as a chlorhexidine mouthwash or mechanical cleaning such as with a toothbrush.

In various embodiments, the therapeutic formulation is administered topically, as often as required, usually once or twice daily. In various embodiments, the composition is administered topically via a cream, serum, deodorant, spray, or moisturizer. It may be recommended to pre-treat the skin with water, soap, or a cleansing formulation prior to administration.

In various embodiments, the therapeutic formulation is administered rectally or vaginally, as required, usually once or twice daily via pessary or suppository.

In various embodiments, the therapeutic formulation is administered via a pulmonary route e.g. by nebuliser or inhaler, as often as required, usually once or twice daily.

The therapeutic formulation is useful for improving the oral health of a subject. For example, by preventing or treating any of the conditions identified in WO2001027143, WO2002070719, and WO2005007178 (supra), and all incorporated herein by reference in their entireties. S. salivarius M18 is also known to help reduce dental plaque, support oral health and oral flora, reduce dental caries, prevent dental caries, treat and prevent gingivitis, and treat and prevent periodontitis (Burton, J. P., et al., 2013 J Med. Microbiol. 62, 875-884; Burton, J. P., et al., 2013, PLoS ONE 8.; Di Pierro, et al. 2015. Clin Cosmet Investig Dent.; 7:107-13; L Scariya, D. V, N., M Varghese, 2015. Int. J. Pharma Bio Sci. 6, 242-250).

### Method of Manufacturing Ingredient

In one aspect, the invention provides a method of manufacturing a composition comprising S. salivarius and an effective amount of a supplemental saccharide, the method comprising:
(a) combining S. salivarius with supplemental saccharide, and
(b) mixing to produce a homogeneous blend;
wherein the S. salivarius is S. salivarius M18, S. salivarius K12, or a combination thereof, and
wherein the supplemental saccharide is galactose or raffinose or a combination thereof.

In various embodiments, the Streptococcus salivarius is in the form of a powder. In various embodiments, the saccharide is in the form of a powder, for example a freeze-dried powder. In various embodiments the powder is a raw ingredient powder comprising the S. salivarius and lyoprotectant as discussed above.

In various embodiments, the mixing occurs in a blender. In various embodiments, the product is then packaged.

In various embodiments, excipients are added.

In various embodiments, the inhibitory profile of the S. salivarius in the composition is improved.

In various embodiments, the composition is for use in the treatment or prevention of a disease or disorder, including the diseases and disorders referenced above.

In various embodiments, the composition is for use in inhibiting a microbial population sensitive to a Blis-producing S. salivarius.

In one aspect, the invention relates to the use of a composition manufactured by the method of the invention for the treatment or prevention of a disease or disorder.

In one aspect, the invention relates to the use of a composition manufactured by the method of the invention for inhibiting a microbial population sensitive to a Blis-producing S. salivarius.

### Method of Manufacturing Formulations

In one aspect, the invention provides a method of manufacturing a therapeutic formulation comprising a composition comprising S. salivarius and an effective amount of a supplemental saccharide, the method comprising
a) mixing excipients,
b) adding a composition comprising S. salivarius and supplemental saccharide of the invention, and
c) blending to provide the therapeutic formulation
wherein the Streptococcus salivarius is Streptococcus salivarius M18, S. salivarius K12, or a combination thereof, and
wherein the supplemental saccharide is galactose or raffinose or a combination thereof.

The method may include further processing steps to provide the therapeutic formulation. A skilled worker will appreciate the required excipients depending on the administration route and blending methods such as homogenisation.

In various embodiments, the invention provides a method of manufacturing an oral lozenge comprising a composition comprising S. salivarius and an effective amount of a supplemental saccharide, the method comprising
a) mixing a carrier, tableting aids (e.g. binder, lubricant), and flavouring agent,
b) adding a composition comprising S. salivarius and supplemental saccharide of the invention, and
c) blending the mixture, and
d) lozenging the mixture to provide the lozenge
wherein the S. salivarius is S. salivarius M18, S. salivarius K12, or a combination thereof, and
wherein the supplemental saccharide is galactose or raffinose or a combination thereof.

The inventors have determined that the supplemental saccharides investigated herein do not affect the stability of S. salivarius in the lozenges (data unreported).

In various embodiments, the invention provides a method of manufacturing an oral powder comprising a composition comprising S. salivarius and an effective amount of a supplemental saccharide, the method comprising
a) mixing a carrier, tableting aids (e.g. binder, lubricant), and flavouring agent,
b) adding a composition comprising S. salivarius and supplemental saccharide of the invention, and
c) blending the mixture to provide the oral powder
wherein the S. salivarius is S. salivarius M18, S. salivarius K12, or a combination thereof, and
wherein the supplemental saccharide is galactose or raffinose or a combination thereof.

In various embodiments, the invention provides is a method of manufacturing a topical composition comprising a composition comprising S. salivarius and an effective amount of a supplemental saccharide, the method comprising
a) mixing an oil vehicle and dispersing agent,
b) adding a composition comprising S. salivarius and supplemental saccharide of the invention, and
c) homogenising the mixture to provide the topical composition,
wherein the S. salivarius is S. salivarius M18, S. salivarius K12, or a combination thereof, and
wherein the supplemental saccharide is galactose or raffinose or a combination thereof.

In various embodiments, the invention provides a method of manufacturing a pessary or suppository comprising a composition comprising Streptococcus salivarius and an effective amount of a supplemental saccharide, the method comprising
a) mixing a solid lipid with other excipients,
b) adding a composition comprising S. salivarius and supplemental saccharide of the invention, and
c) homogenising the mixture to provide the pessary or suppository,
wherein the S. salivarius is S. salivarius M18, S. salivarius K12, or a combination thereof, and
wherein the supplemental saccharide is galactose or raffinose or a combination thereof.

In various embodiments, the invention provides a method of manufacturing a formulation for pulmonary administration comprising a composition comprising S. salivarius and an effective amount of a supplemental saccharide, the method comprising
a) optionally mixing a dry powder carrier with other excipients,
b) adding a composition comprising S. salivarius and supplemental saccharide of the invention, and
c) mixing to provide the formulation for pulmonary administration,
wherein the S. salivarius is S. salivarius M18, S. salivarius K12, or a combination thereof, and
wherein the supplemental saccharide is galactose or raffinose or a combination thereof.

The following non-limiting examples are provided to illustrate the present invention and in no way limit the scope thereof.

### EXAMPLES

### Materials

The following culture media and saccharide were all supplied by Fort Richard Laboratories, New Zealand: CABK12 agar plates; Columbia Blood Agar Base (CAB) bottles; Colombia agar base with 0.5% (w/v) CaCO₃ (CABCa). Columbia agar Base supplemented with 0.1% CaCO₃ (and 5% v/v sheep blood (sBaCa); Haemophilus agar plates Mitis Salivarius Agar; D-galactose—BD Difco; Todd Hewitt Broth (THB)—BD Difco (used as per manufacturers instruction); M17 Broth—BD Difco (used as per manufacturers instruction, but no saccharide added).

The following materials were all supplied by Lab Supply Ltd, New Zealand: Calcium carbonate (CaCO₃)—PanReac Applichem; D-(+) Glucose—monohydrate—Applichem; Ethanol (96% AR Grade)—diluted with water to produce 70% solution prior to use. D (+)-Glucose monohydrate—Applichem, Chloroform (Emsure, ACS, ISO, Reag. Ph Eur)—Merck. Water for molecular biology (Nuclease free)—AppliChem, 10× Tris-Acetate-EDTA (TAEl) buffer—Applichem. Hydrochloric acid (HCl).

D-(+) Raffinose pentahydrate; D-(−) Fructose (both from Sigma, New Zealand).

Chloroform (Emsure, ACS, ISO, Reag. Ph Eur)—Supelco (Sigma Aldrich, New Zealand).

The following materials were all supplied by Thermo Fisher Scientific, New Zealand: PureLink RNA Mini Kit; TRIzol reagent; Phasemaker tubes; RNaseZap; TURBO DNA-free kit; SuperScript IV VILO; PowerTrack SYBR Green master Mix; 10,000×SYBR safe DNA gel stain; MicroAmp Optical 384-Well reaction plate; MicroAmp Optical adhesive film and Phosphate Buffered Saline (PBS)—Oxoid

Primers for qPCR:
K12recA_fw
TGAGTCAAGCCATGCGTAAG
K12recA_rv
CAGGGGTCGTTTCTGGATTA
K12gyrA_fw
(5'-CCGTTACATGTTGGGTTGACG)-
K12gyrA_rv
(5'-ATCACGAAGCATTTCCAAGG)
18K12salA_fw
TGATTGCCATGAAAAACTCAA
M18K12salA_rv
TTGCAAACCAACCTGTACCTC
K12SalQ_fw
ACACTCGATCTCGAGGCACT
K12salQ_rv
GAATGTGGCATAACCAACTA
K12salB_fw
CTCGTTGAGATTGAAGCAATGA
K12salB_rv
TTCGTGTGAAATGGTTTGGA
K12ureC_fw
TTTCCAAGGTCGTACCGTTC
K12ureC_rv
TGGATCCAAGTGGTGACAAA
Primers for PCR:
SalBF
GTGAATTCTCTTCAAGAATTGACTCTT
SalBR
AAAATATTCATACCGCTCTTCC

Phosphate Buffered Saline (PBS)—Dulbecco A—Oxoid (supplied by Thermo Fisher Scientific, New Zealand and used as per manufacturer's instructions). AnaeroGen sachets—Oxoid—supplied by Thermo Fisher Scientific, New Zealand.

Promega GoTaq G2 Hot start Green Master mix supplied by Invitro Technologies, New Zealand.

Maestrogen AccuRuler 1 kb DNA RTU ladder supplied by Mediray, NZ.

Similac 360 Total Care infant formula (Abbott Global, US) was purchased via www.Amazon.com. Whole milk powder (Anchor Blue™ Milk powder, Anchor, NZ.)

S. salivarius strains K12 and M18—available from Deutsche Sammlung von Mikroorganism und Zellkulturen (DSM), sourced from Blis Technologies Ltd, New Zealand; K12 raw ingredient product (K12 powder with trehalose/lactitol/maltodextrin), M18 raw ingredient product (M18 powder with trehalose/lactitol/maltodextrin); K12 and M18 Dairy free raw ingredient products (powder with sucrose, sodium citrate, and maltodextrin lyoprotectant); K12 containing commercial powder formulation, Daily Defence Junior (DDJ) (Composition: S. salivarius K12, Isomalt, Maltodextrin, Vanilla flavour) K12 containing commercial lozenge formulation (Throat Guard Pro) (composition: S. salivarius K12, isomalt, tableting aids, natural flavour)—all from Blis Technologies Ltd, New Zealand); S. saprophyticus ATCC15305, S. mutans NCTC 10449 (ATCC 25175); S. mutans UA159 (ATCC700610); S. cohnii (ATCC29974); S. simulans (ATCC 27848); C. acnes 6919; F. nucleatum ATCC 25586; P. gingivalis ATCC 33277; P. gingivalis ATCC 53978; P. intermedia ATCC 25611; S. salivarius (ATCC 13419, ATCC25923 and ATCC 7073); S. sobrinus ATCC 27351 S. agalactiae ATCC 12386, A. viscosus ATCC 15987 C. auris ATCC 51966—all available from American Type Culture Collection (ATCC); M. catarrhalis TW1 and TW2; L. lactis T-21; S. pyogenes M76; P. aeruginosa 12; H. influenzae TW5; S. aureus A222; S. mutans OMZ175; S. pneumoniae D39; S. pyogenes 71-698; S. constellatus T-29; S. pyogenes FF22; S. pyogenes W-1; S. pyogenes M17; S. pyogenes M57; S. pyogenes EMM92; S. pyogenes M66; S. pyogenes M74; S. pyogenes H13; S. pyogenes K26; S. pyogenes WS02, S. dysgalactiae Bris 2; S. dysgalactiae T277; S. pneumoniae RX1; S. pneumoniae PK8; S. mutans D10; S. mutans FW75; A. viscosus T14; S. sanguis K11; S. aureus 20; S. aureus 19; S. aureus 14; F. nucleatum FH2; F. nucleatum FH3; S. salivarius CN3410; 34A; K14; H29; S. pyogenes K7; S. dysgalactiae T-148; S. aureus 19—all available from Blis Technologies Ltd on request. S. sobrinus OMZ176 (CCUG21020)—available from Culture Collection University of Gothenburg (CCUG); S. pyogenes 71-679—standard gifted from Lewis Wannamaker.

### Method 1—Preparing Solid Culture Media

Calcium carbonate (CaCO₃) were added to all solid culture media (CAB) during preparation as a buffering agent. CaCO₃ was present in all prepared culture media at a 0.5% (w/v) concentration (CABCa).

Premade bottles of Columbia agar base (CAB) agar were ordered from Fort Richard Laboratories, New Zealand in 180 mL volumes. Additional ingredients were added to these 180 mL bottles. Carbohydrates were added to separate bottles of CAB agar at 0.1%, 0.5%, 1.0%, 1.5%, and 2.0% (w/v) concentrations. The following equation was used to determine the amount of carbohydrate (and CaCO₃) to add to achieve a desired concentration of that ingredient:

$$Volume of Dissolvent ( mL ) \times ( \frac{( desired contraction ( \% ) ( \frac{w}{v} ) )}{100} ) = amount of solute to add ( g )$$

For example, to obtain a CAB agar base containing CaCO₃ and galactose at a 0.5% (w/v) concentration using one of the premade Fort Richard agar bottles the following equation would be used:

$$180 ( mL ) = ( \frac{0.5 ( \% ) ( \frac{w}{v} )}{100} ) = 0.9 ( g )$$

A precise method was used to ensure sterility of the culture media and that accurate amounts of any additive ingredients were correctly integrated into the culture medium. Ingredients were removed from their containers using clean spoons/spatulas and were weighed in clean weigh boats on a balance which measured to the milligram. Once the ingredients had been weighed out, they were tipped into clean 30 ml containers, labelled, and sealed. Premade CAB agar is solid when it arrives from Fort Richard, therefore a crater approximately 3 cm wide and 3 cm deep must be cut into the agar using a sterile scalpel to create a space for the ingredients to be poured into. Following this the ingredients were poured into the crater and 1 ml of sterile distilled water was dispensed on top to partially soak the ingredients into the agar. Agar that was cut away to create the cavity was then replaced within the crater and a magnetic stirrer is placed into the bottle. The bottle was then autoclaved at 110° C. for 10 minutes. The bottle was stored in a water bath set to 50° C. Sterile petri dishes were labelled with the appropriate information (date, contents of the plate, and added ingredients) and placed into the Class II Biological Safety Hood with their lids off. Prior to pouring the agar into the petri dishes, the contents were stirred thoroughly for 1-2 minutes using the magnetic stirrer. A Bunsen flame was lit inside the Biological Safety Cabinet as a further sterility measure and the contents of the bottle was poured out into the petri dishes. If any bubbles appeared while pouring, they were popped using the Bunsen flame. The agar was left for 5-10 minutes to set. One plate was labelled “negative control” and placed into the incubator overnight to ensure no contamination occurred during agar preparation.

### Method 2—Deferred Antagonism Assay

1-2 producer strain colonies were transferred from a stock plate into 900 μL of suspension solution using a fresh cotton swab. Following this, suspensions were vortexed, and a sample was inoculated onto solid culture media using a fresh cotton swab. The inoculation was a streak that runs diametrically across the agar plate in a 1.5 cm wide strip. The plate was then incubated. Following incubation all visible colonies were removed by swabbing them from the agar surface. The plates were then treated with chloroform by dispensing 2 ml of chloroform onto 4 cm×4 cm piece of cloth and sealing the inverted agar plate on top for 30 minutes. The plates were then air dried for 30 minutes following chloroforming. 1-2 indicator strain colonies were then transferred from a stock plate into complementary suspension solution. The suspensions were then vortexed, and a sample was inoculated onto the same agar surface. These samples were streaked in a perpendicular direction to the initial producer streak.

Each indicator streak was prepared with fresh a suspension containing cells from a corresponding stock plate containing the indicator strain of interest. Indicator strains suspension samples were transferred using a fresh cotton swab. Results were measured by qualitatively reviewing the subsequent (if any) sizes of the zones of inhibition (ZOI) of indicator strain colony growth. A positive result is when there is a distinctive reduction or complete inhibition of indicator colony growth on the culture medium. A negative result is when there is no suggestion of a reduction in the indicator strain colony density.

### Method 3—Dose Response Assay

CABCa agar plates containing various concentrations of saccharide were prepared according to the methods described in Method 1. Producer strains were transferred from stock agar plates into 900 μL THB using a fresh cotton swab. A plastic spread plater was cut to approximately 1.2 cm width and then dipped into ethanol and left to air dry to ensure sterility. A 100 μL sample of the producer strain suspension was then dispensed onto the agar surface in a straight line running diametrically across the agar surface in (approximately) 20 μL spots. The spots were spaced approximately 1.5 cm apart from each other. The plastic spreader was then used to evenly distribute the spots across the agar surface in a 1.5 cm wide strip. This step was taken to ensure an even producer streak was inoculated onto the agar surface and provided an approximate measure of the initial inoculum concentration. Separate plastic spreaders were used for samples containing different producer strains. The samples were left to soak into the agar surface until there was no visible liquid remaining on the agar surface (approximately 20 minutes). Following producer streak preparations, the deferred antagonism assay protocol was followed as per Method 2. Results were recorded by measuring the size of the zone of inhibition (ZOI) in mm.

### Example 1: Antagonism of ENTR Microorganisms by K12

This example shows inducement of antagonism of ENTR microorganisms by K12 and supplemental saccharide.

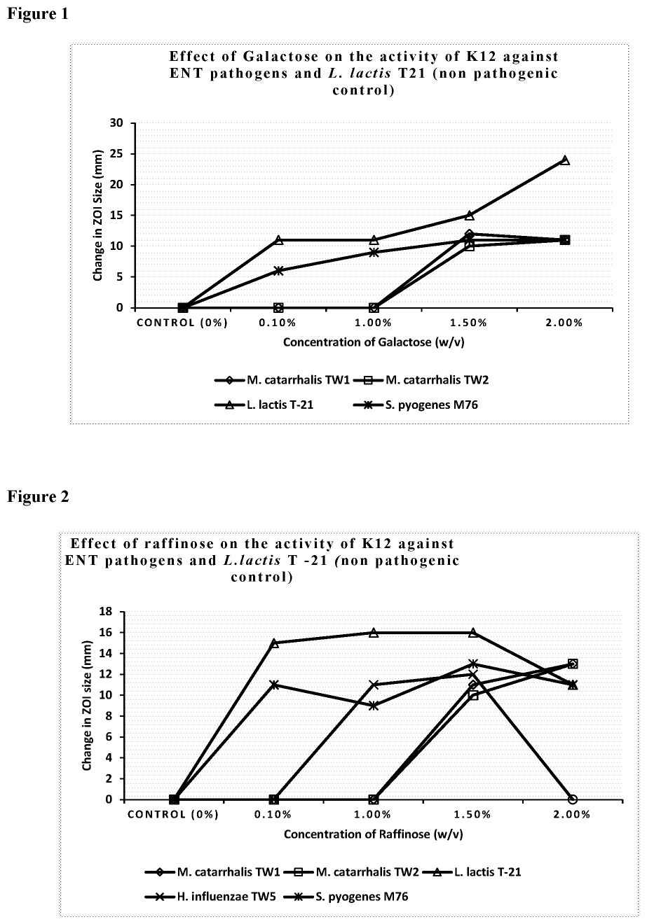

BLIS K12™ cultured onto CABCa agar plates containing 0.1%, 1.0%, 1.5%, and 2.0% (w/v) concentrations of galactose and raffinose were studied for their antimicrobial activity by a deferred antagonism assay according to the protocols outlined in Method 3. The results presented in FIG. 1 and FIG. 2 illustrate the change in ear, nose, and throat (ENT) microorganism indicator strain zone of inhibition sizes. When K12 was cultured in the presence of various concentrations of galactose the ZOI sizes for several of the ENTR indicator strains increased markedly from the control condition (FIG. 1). M. catarrhalis TW1 and TW2 display similar changes in the ZOI sizes across the various concentrations of galactose. The change in TW1 ZOI size peaks at 1.5% galactose(w/v) (12 mm) then decreases at 2.0% galactose (w/v), whereas the change of TW2 ZOI size peaks at 2.0% galactose (w/v) (11 mm). The change in L. lactis T-21 ZOI sizes trend upwards relative to the concentration of galactose present in the culture medium.

S. pyogenes M57 ZOI sizes increase from the control condition at 0.1%, 1.0%, and 1.5% (w/v) concentrations of galactose. The change in M76 ZOI size remains level (11 mm) between 1.5% and 2.0% concentrations of galactose. The concentration of galactose that produces the largest ZOI change (on average) is 2.0% (w/v) (Average ZOI change @ 2% galactose=9.5 mm).

When K12 was cultured on CABCa incorporated with various concentrations of raffinose during a deferred antagonism assay, a change in the ZOI sizes from a control condition (no carbohydrate) was demonstrated for several ENT microorganism indicator strains (FIG. 2). The change in L. lactis ZOI size from the control condition to 0.1% raffinose (w/v) was 15 mm. This large increase was followed by an increase to 16 mm at 1.0% raffinose (w/v) (indicating significantly greater inhibition), where the ZOI size then levels off and eventually decreases at 2.0% raffinose (w/v). The change in ZOI size for H. influenzae TW5 and S. pyogenes M76 also peaked at 1.5% raffinose (w/v) at 12 mm and 13 mm, respectively. TW5 ZOI size actually returned to the same as the control condition (0 mm) at 2% raffinose (w/v). M. catarrhalis TW1 and TW2 ZOI sizes changed very similarly between conditions (FIG. 2). The changes in ZOI size for each strain peak at the end of our range of study (2.0% (w/v) (13 mm). The concentration of galactose that produced the largest ZOI size change (on average) was the 1.5% raffinose (w/v) condition (Average ZOI change @ 1.5% raffinose=10.33 mm).

### Example 2: Antagonism of ENTR Microorganisms by M18

This example shows inducement of antagonism of ENTR microorganisms by M18 and supplemental saccharide.

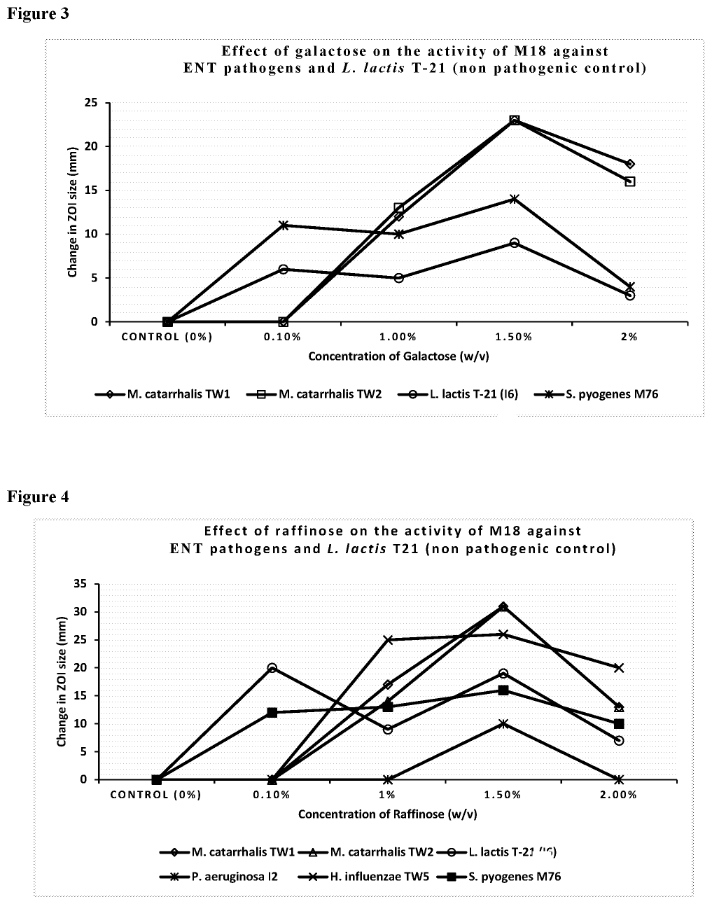

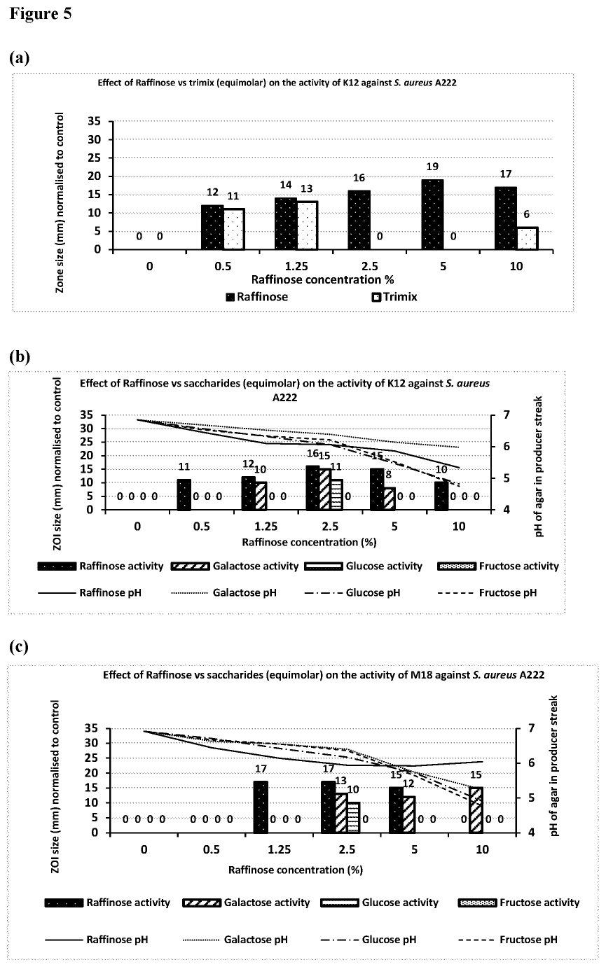

BLIS M18™ cultured onto CABCa agar plates containing 0.1%, 1.0%, 1.5%, and 2.0% (w/v) concentrations of galactose and raffinose were studied for their antimicrobial activity by conducted a deferred antagonism assay according to the protocols outlined in Method 3. The results presented in FIG. 3 and FIG. 4 illustrate the change in ear, nose, and throat (ENT) microorganism indicator strain zone of inhibition sizes.

When M18 was cultured on CABCa incorporated with various concentrations of galactose there was a subsequent change in the ZOI sizes for several ENTR microorganism indicator strains during a deferred antagonism assay (FIG. 3). M. catarrhalis strains TW1 and TW2 both demonstrated increases in their respective zones of inhibition when galactose was incorporated in the culture media at 1.0% and 1.5% (w/v) concentrations versus the control condition (12+23 mm and 13+23 mm, respectively). Both L. lactis and S. pyogenes M76 zones of inhibition were the largest when galactose was present at a 1.5% (w/v) concentration (9 mm and 14 mm, respectively). These were marked differences from their control condition ZOI sizes. All susceptible ENTR indicator strains showed a reduced ZOI size when galactose was present at a 2.0% (w/v) concentration compared to the 1.5% (w/v) concentration condition. The condition which produced the largest ZOI change, on average, was the 1.5% galactose (w/v) condition. Average ZOI change @ 1.5% galactose=11.5 mm.

When M18 was cultured on CABCa agar incorporated with various concentrations of raffinose a very similar pattern emerged to what was observed in ZOI sizes in the galactose conditions. However, in the raffinose conditions, more ENTR indicator strains exhibited some inhibition and the ZOI sizes (on average) were greater than the corresponding ZOI sizes in the galactose conditions. M. catarrhalis strains TW1 and TW2 zones of inhibition displayed the largest change in size from the control condition when raffinose was present at a 1.5% (w/v) concentration (31 mm, respectively). Contrary to the trend observed in the galactose condition, L. lactis ZOI size was greatest in the 0.1% (w/v) raffinose condition (20 mm). P. aeruginosa I2 ZOI size did not change from the control condition across any of the raffinose conditions, excluding at 1.5% (w/v) concentration where the ZOI=10 mm in width. H. influenzae TW5 ZOI size was greatest in the 1.5% (w/v) raffinose condition (26 mm). Again, all susceptible ENTR indicator strain ZOI sizes exhibited marked reductions in width when raffinose was incorporated into the culture media at a 2.0% (w/v) concentration (FIG. 4). The condition which produced the largest ZOI change, on average, was the 1.5% raffinose (w/v) condition. Average ZOI change @ 1.5% raffinose=22.16 mm.

When the same experiment was conducted with K12−/− (a strain that does not have the bacteriocin encoding megaplasmid) there were no zones of inhibition formed for any of the ENTR indicator strains.

### Example 3: Activity of Raffinose Compared to Trimix (Mixture of Equal Molar Concentrations of the Three Saccharides) and Individual Saccharides

This example shows that the stimulatory effect of K12 and M18 was due to raffinose and not to equimolar amounts of one or all of its individual constituents, i.e. the raffinose is not being metabolised to individual constituents that are having effect. Further to this, the effect of any change in pH from the assay design could have had in influencing the change in inhibitory effects was assessed.

A dose response assay was carried out according to method 3 above, except a 1.2 cm streak was used.

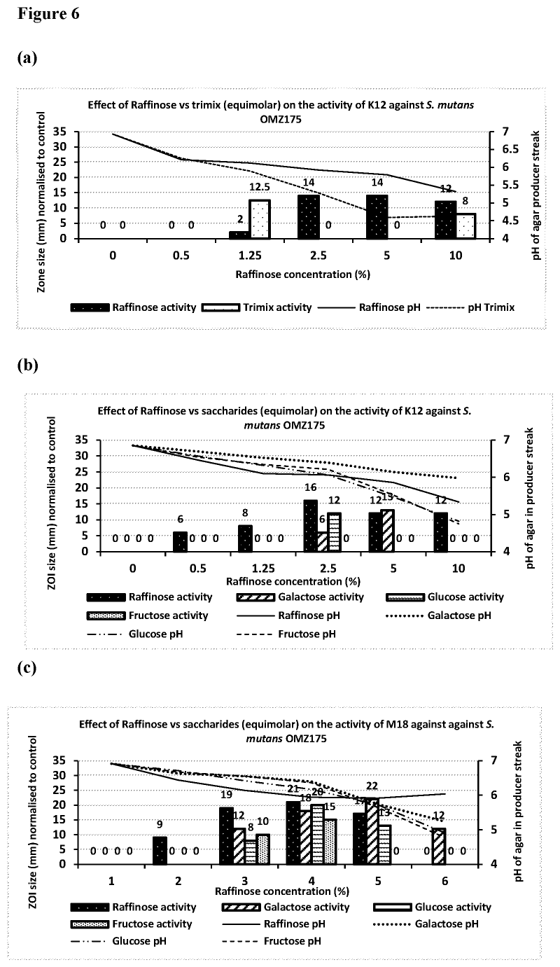

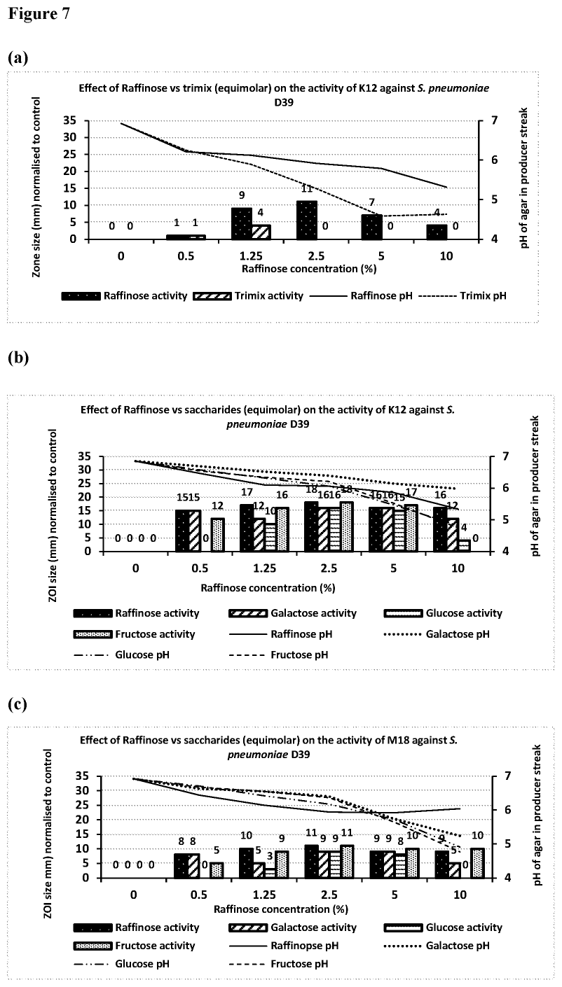

FIGS. 5-7 highlight the enhanced efficacy of BLIS K12 or M18±raffinose, trimix and individual saccharides against skin, dental, ENTR, pathogens.

Conclusions: The comparative assessment of equimolar concentration of raffinose vs trimix saccharides and individual saccharides showed that in presence of raffinose, K12 has better inhibitory activity against microorganisms associated with skin, dental, ENTR, compared to an equivalent composition of the three monomeric saccharides. The effect seems to be mediated via enhanced production of antimicrobial molecules (such as bacteriocins or non-ribosomal peptides) and not an inhibitory effect due to drop in pH.

### Example 4: Activity of Raffinose Compared to Trimix (Mixture of Equal Weight Percentage Concentrations of the Three Saccharides) and Individual Saccharides

This example shows that the stimulatory effect of K12 and M18 was due to raffinose and to equal percent weight amounts of one or all of its individual saccharides, i.e. the raffinose is not metabolised to individual saccharides that are having this effect.

A dose response assay was carried out according to method 3 above, except a 1.2 cm streak was used. For the individual saccharide testing the pH of the producer streak was adjusted after the growth of producer to change the acidic conditions.

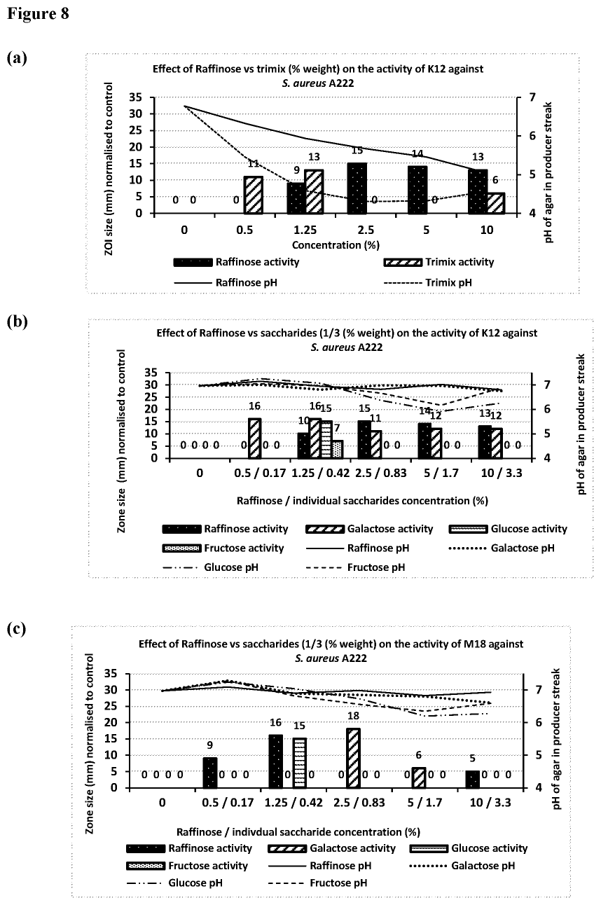

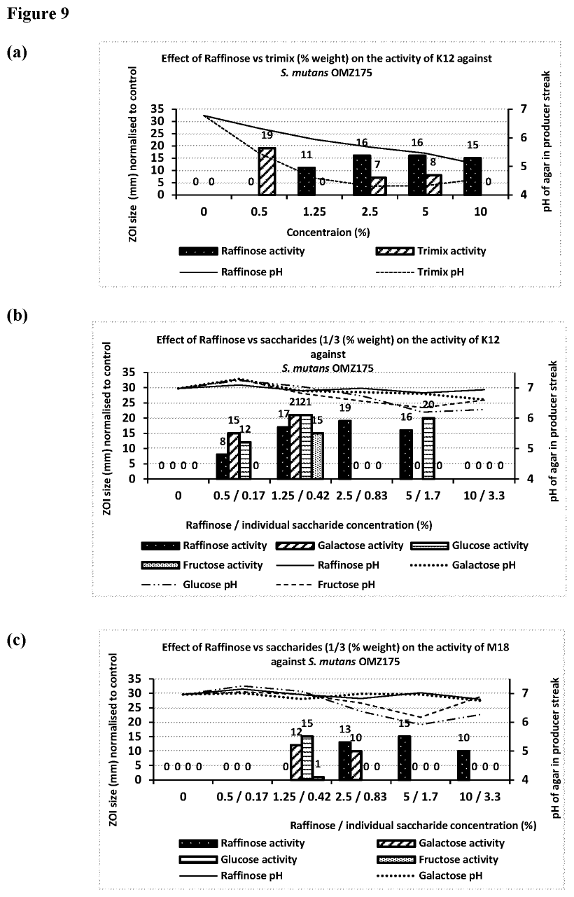

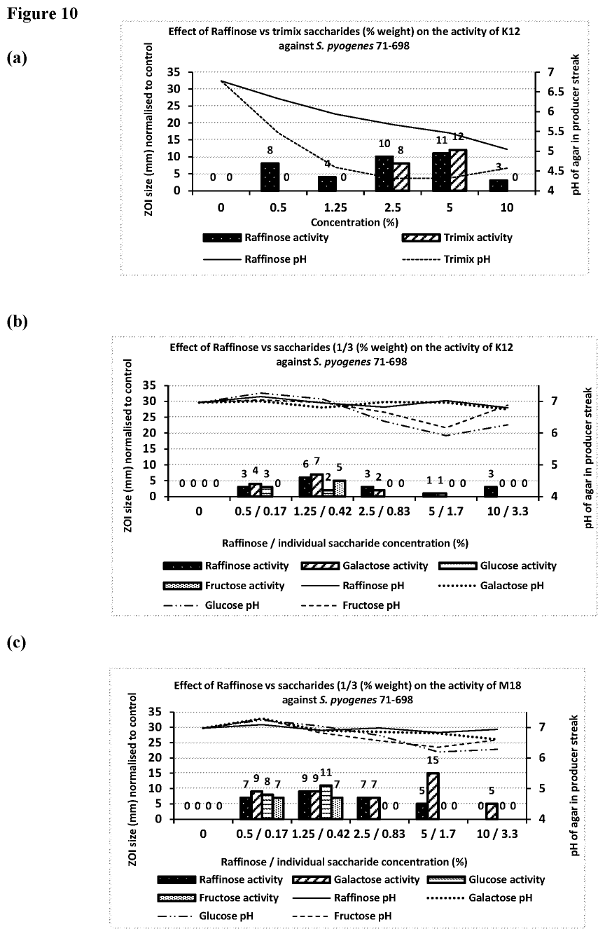

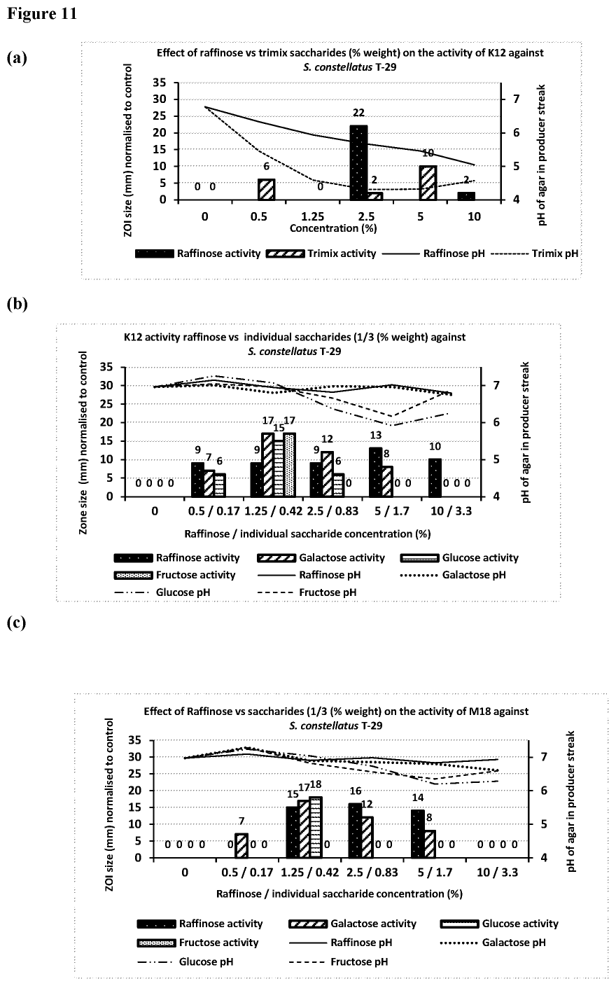

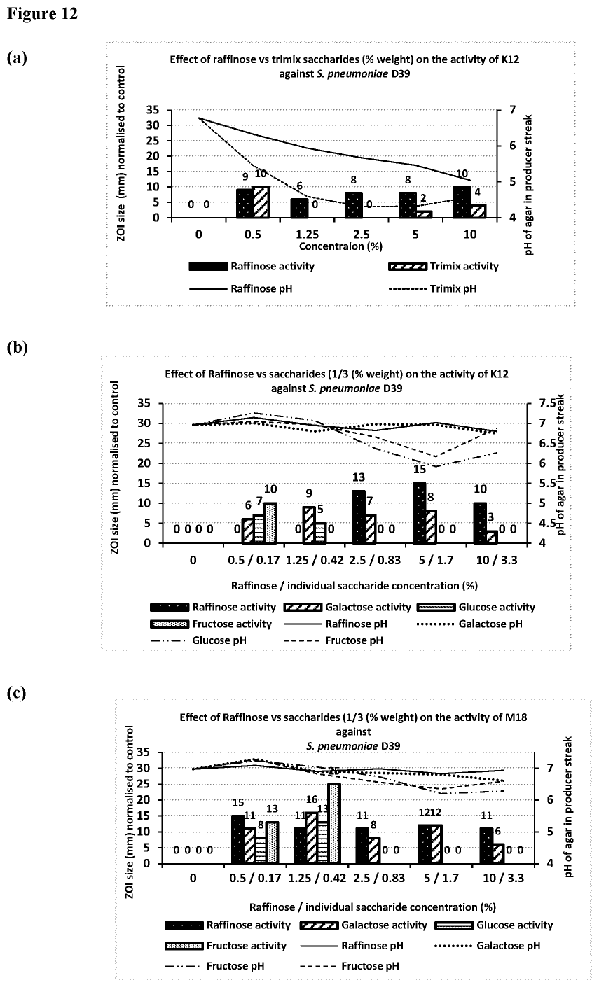

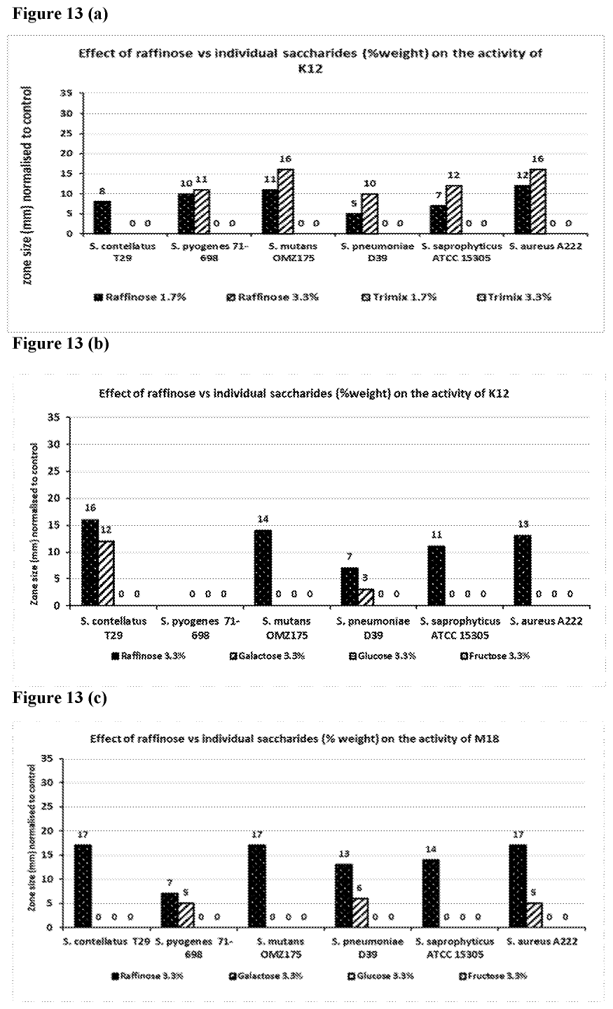

The enhanced efficacy of K12 or M18+(% w/v) with raffinose over an equal weight percentage amount of saccharides either as a trimix or as individual saccharides was seen against representative microorganism from skin, dental, lower respiratory diseases as well as other S. salivarius (FIG. 8-13). The activity is normalised to baseline control.

### Example 5: Raffinose Promotes Antimicrobial (e.g. Bacteriocin or Non-Ribosomal Peptides) Activity of K12 and M18 Rather than Acid Production

The example shows that the stimulation of greater inhibition of these microorganisms was due to an increase in production of antimicrobial molecules (bacteriocins or non-ribosomal peptides) and not due to an associated decrease in pH to a more (and therefore potentially inhibitory) acidic environment.

Method: Bacterial test strains were assessed for their ability to grow on an equivalent agar to Columbia agar base without calcium carbonate at different pH's ranging from pH 4.5 to pH 7. The bacterial test strains were suspended in either Todd Hewitt broth or a relevant growth media for the strain, before being swabbed across the test plates at different pH's. These plates were then incubated for at 37° C. with 5% CO₂, bacterial growth for each test strain was monitored at 18 and 24h.

Results: At 24h all the strains tested grew at pH 7 but as the pH dropped the number of strains able to grow decreased (Table 1). Only 3 out of the 9 strains grew at pH 5.25 or below. This shows that production of acid by either K12 or M18 grown on CABCa supplemented with trimix or individual saccharides could either reduce or totally inhibit the growth of the strains being tested.

TABLE 1

| | | pH | | | | | | | | |
|---|---|---|---|---|---|---|---|---|---|---|
| Indicator microorganism | strain | 4.5 | 4.75 | 5 | 5.25 | 5.5 | 5.75 | 6 | 6.5 | 7 |
| 1. S. saprophyticus | ATCC 15305 | − | LG | + | + | + | + | + | + | + |
| 2. S. mutans | OMZ175 | − | LG | LG | LG | + | + | + | + | + |
| 3. S. pneumoniae | D39 | − | − | − | − | − | − | LG | LG | + |
| 4. S. constellatus | T-29 | − | − | − | LG | LG | + | + | + | + |
| 5. S. pyogenes | 71-698 | − | − | − | − | + | + | + | + | + |
| 6. H. influenzae | TW5 | − | − | − | − | − | − | − | − | + |
| 7. M. catarrhalis | TW1 | − | − | − | − | − | − | − | + | + |
| 8. S. aureus | A222 | + | + | + | + | + | + | + | + | + |

− = no growth,
+ = growth,
LG = less growth
Note that raffinose did not induce significant drop in pH (>5.25) but results in inducing K12 to produce inhibitory activity. Observed inhibitory activity of K12 in presence of raffinose is due to antimicrobial activity, not a pH effect.

### Example 6: Activity of Raffinose with K12 or M18 Compared to Trimix (Mixture of Equal Weight Percentage Concentrations of the Three Saccharides) and Individual Saccharides

This experiment shows that the inhibitory spectrum of K12 was found to be extended to species and strains, not typically inhibited by K12 (Table 2 and 3). K12 was induced by raffinose specifically against skin microorganisms, highlighting the potential benefit of K12 to skin indications such as impetigo, atopic dermatitis etc.

The method used was the same as Example 4.

Results The below Table 2 highlights which species/strains became sensitive to the inhibitory molecules (e.g. bacteriocins) from K12 that has been incubated with raffinose (Table 2). Considering for example 2.5% raffinose would contain equal percentage (0.83% each of the three saccharides). Similar results were obtained for M18.

TABLE 2

| | Inhibitory activity of S. salivarius K12 in presence of saccharides | | | | | |
|---|---|---|---|---|---|---|
| | No saccharide | Raffinose (Range: | Trimix (Range: | Galactose (Range: | Fructose (Range: | Glucose (Range: |
| Indicator strains | (Control) | 2.5%-10% w/v) | 2.5%-10% w/v) | 0.83%-3.3% w/v) | 0.83%-3.3% w/v) | 0.83%-3.3% w/v) |
| S. aureus A222 | x | ✓ | x | ✓ | x | x |
| S. saprophyticus ATCC 15305 | x | ✓ | x | x | x | x |
| S. mutans OMZ175 | x | ✓ | ✓ | x | x | x |
| Total strains inhibited | 0/3 | 3/3 | 1/3 | 1/3 | 0/3 | 0/3 |

This effect shown in Table 3 below was seen at a nominated percentage of raffinose and equivalent trimix (by percent mass) or individual saccharides

TABLE 3

| | Inhibitory activity of S. salivarius K12 in presence of saccharides | | | | | |
|---|---|---|---|---|---|---|
| | No saccharide | Raffinose | Trimix | Galactose | Fructose | Glucose |
| Indicator strains | Control | (3.3% w/v) | (3.3% w/v) | (3.3% w/v) | (3.3% w/v) | (3.3% w/v) |
| S. aureus A222 | x | ✓ | x | x | x | x |
| S. saprophyticus ATCC 15305 | x | ✓ | x | x | x | x |
| S. mutans OMZ175 | x | ✓ | x | x | x | x |
| Total strains inhibited | 0/3 | 3/3 | 0/3 | 0/3 | 0/3 | 0/3 |

K12 or M18 in presence of raffinose showed enhanced potency when PGP-27 T2 compared with equimolar or equal percentage weight concentrations of trimix saccharides or saccharides alone.

The inhibitory activity of raffinose is attributed to induction of K12 or M18 to produce bacteriocin rather than or in addition to acid production due to metabolism of simple saccharides (acid production in the oral cavity can cause dental caries and provide safe niche for acid tolerant bacteria such as S. mutans).

A selective dose related inhibitory effect was observed with raffinose suggesting a specific dose level of raffinose is required to achieve the benefits rather than a blanket prebiotic effect with common saccharides alone or in combination.

### Specific Observations:

Indicator strain S. aureus A222 went from being insensitive to K12 to being sensitive once 1.25, 2.5, 5 and 10% w/v raffinose was added to K12.
Indicator strains S. mutans OMZ175, and S. saprophyticus ATCC 15305 went from being insensitive to K12 to being sensitive once 2.5, 5 and 10% w/v raffinose was added.
Indicator strain S. mutans OMZ175 went from being insensitive to M18 to being sensitive once 0.5, 1.25, 2.5, and 5% w/v raffinose was added.
Indicator strains S. constellatus T-29, S. aureus A222, and S. saprophyticus ATCC 15305 went from being insensitive to M18 to being sensitive once 1.25, 2.5, 5% w/v raffinose was added.

An increase in inhibition by K12 was seen for:
S. constellatus T-29 with 0.5 to 10% w/v raffinose
S. pyogenes with 0.5 to 10% w/v raffinose
S. pneumoniae with 0.5 to 10% w/v raffinose

An increase in inhibition by M18 was seen for:
S. pyogenes with 0.5 to 10% w/v raffinose
S. pneumoniae with 0.5 to 10% w/v raffinose

### Example 7A: Effect of Concentration of Raffinose to Induce Inhibitory Activity of K12 or M18

This example shows the effect of concentration of raffinose for enhanced inhibitory effects. Raffinose concentrations of 1.7, 2.5, 3.3, 5 and 10% were compared in Table 4-7 below.

Blank spaces in the table are where no result was obtained due to contamination of the test.

TABLE 4
K12

| | | | | | | | | | | Total |
|---|---|---|---|---|---|---|---|---|---|---|
| | | | S. | S. | S. | S. | S. | M. | S. | number of |
| | | | constellatus | pyogenes | mutans | pneumoniae | saprophyticus | catarrhalis | aureus | strains |
| Producer | Saccharide | % wt | T29 | 71-698 | OMZ175 | D39 | ATCC 15305 | TW1 | A222 | inhibited |
| K12 | Raffinose | 1.7% | 8 | 10 | 11 | 5 | 7 | 0 | 12 | 6 |
| | | 2.5% | 22 | 9 | 16 | 8 | 12 | 10 | 15 | 7 |
| | | 3.3% | — | 11 | 16 | 10 | 12 | 0 | 16 | 5 |
| | | 5% | — | 7 | 16 | 8 | 13 | 5 | 14 | 6 |
| | | 10% | 2 | 3 | 15 | 10 | 12 | 6 | 13 | 6 |

TABLE 5
K12 (pH adjusted to counter inhibitory effect due to acidic pH)

| | | | | | | | | | | Total |
|---|---|---|---|---|---|---|---|---|---|---|
| | | | S. | S. | S. | S. | S. | M. | S. | number of |
| | | | constellatus | pyogenes | mutans | pneumoniae | saprophyticus | catarrhalis | aureus | strains |
| Producer | Saccharide | % wt | T29 | 71-698 | OMZ175 | D39 | ATCC 15305 | TW1 | A222 | inhibited |
| K12 | Raffinose | 1.7% | 4 | 4 | 7 | 10 | 0 | 0 | 8 | 5 |
| | | 2.5% | 18 | 3 | 13 | 9 | 8 | 0 | 15 | 6 |
| | | 3.3% | 16 | — | 14 | 7 | 11 | 0 | 13 | 5 |
| | | 5% | 12 | 1 | 15 | 13 | 12 | 0 | 14 | 6 |
| | | 10% | 11 | 3 | 10 | 10 | 11 | 0 | 13 | 6 |

TABLE 6
M18

| | | | | | | | | | | Total |
|---|---|---|---|---|---|---|---|---|---|---|
| | | | S. | S. | S. | S. | S. | M. | S. | number of |
| | | | constellatus | pyogenes | mutans | pneumoniae | saprophyticus | catarrhalis | aureus | strains |
| Producer | Saccharide | % wt | T29 | 71-698 | OMZ175 | D39 | ATCC 15305 | TW1 | A222 | inhibited |
| M18 | Raffinose | 1.7% | 22 | — | 22 | 3 | 16 | 7 | 20 | 6 |
| | | 2.5% | 18 | 8 | 20 | 10 | 17 | 17 | 20 | 7 |
| | | 3.3% | 18 | 8 | 21 | 7 | 17 | 0 | 18 | 6 |
| | | 5% | 16 | 5 | 19 | 2 | 14 | 15 | 16 | 7 |
| | | 10% | 12 | 0 | 0 | 1 | 0 | 0 | 0 | 2 |

TABLE 7
M18 (pH adjusted to counter inhibitory effect due to acidic pH)

| | | | | | | | | | | Total |
|---|---|---|---|---|---|---|---|---|---|---|
| | | | S. | S. | S. | S. | S. | M. | S. | number of |
| | | | constellatus | pyogenes | mutans | pneumoniae | saprophyticus | catarrhalis | aureus | strains |
| Producer | Saccharide | % wt | T29 | 71-698 | OMZ175 | D39 | ATCC 15305 | TW1 | A222 | inhibited |
| M18 | Raffinose | 1.7% | 17 | 10 | 21 | 12 | 15 | 0 | 17 | 6 |
| | | 2.5% | 16 | 7 | 19 | 11 | 15 | 0 | 18 | 6 |
| | | 3.3% | 17 | 7 | 17 | 13 | 14 | 0 | 17 | 6 |
| | | 5% | 14 | 5 | 16 | 12 | 12 | 0 | 13 | 6 |
| | | 10% | 0 | 0 | 0 | 11 | 0 | 0 | 0 | 1 |

Raffinose concentrations of 1.7, 2.5, 3.3 and 5% were all able to inhibit at least 7 of the indicators tested.

Inhibition of indicator M. catarrhalis was seen in Table 6 and but not in Table 7 where the agar pH was re-adjusted after growth of the producer, so this suggests that the inhibition was due to production of acid by the producers.

For most of the indicators, the largest zones of inhibition were measured on the agar plates supplemented with 2.5% raffinose.

### Example 7B: Effect of Concentration of Galactose, Combination of Galactose/Raffinose

Aim: to determine the effect of concentration of galactose to induce the inhibitory activity of K12 and M18.

Method: CABCa agar plates were prepared with or without 0.5, 1.25, 1.7, 2.5, 3.3 and 5% galactose by the addition of 0.5% (w/v) calcium carbonate to solid CAB agar in bottles and then melted by autoclaving at 110° C. for 10 mins. Once cooled, filter sterilised solutions of the saccharides or distilled water were added and mixed and then 20 ml pipetted into petri dishes.

A deferred antagonism assay was conducted using K12 or M18 raw ingredient suspended in Todd Hewitt broth (THB). This was spread as a 1.2 cm streak containing approximately 1-2×10⁶ cfu down the middle of a CABCa control or CABCa galactose supplemented test plate. After 18 hrs growth at 37° C. with 5% CO₂, bacterial growth was removed, and the pH of the agar in the initial streak was measured and adjusted to a pH of 6.5-7.5 using 0.5M sodium carbonate pH11 before sterilizing the agar surface using chloroform vapor. Bacterial test strains were suspended in i-THB before being swabbed across the plates perpendicular to the initial streak. Plates were incubated for a further 18 hrs incubation at 37° C. with 5% CO₂ before measuring (mm) any zones of inhibition for each test strain,

Result and Conclusion: Surprisingly, only at a concentration of 0.5% w/w of Galactose, Blis K 12 and Blis M18 showed enhanced inhibitory activity (potency) and spectrum (number of pathogenic strains) activity against pathogens implicated in ENTR and skin infections (Table 8). This can be concluded that just a mere presence of Galactose may not be enough BUT a specific concentration of Galactose is required to induce the inhibitory activity.

TABLE 8
Effect of different concentrations of Galactose on the inhibitory activity of K12 and M18

| | | Size of zone inhibition normalised to control (mm) | | | | | | | |
|---|---|---|---|---|---|---|---|---|---|
| | | S. | S. | S. | S. | S. | S. | S. | Total |
| | Galactose | constellatus | pyogenes | mutans | pneumoniae | saprophyticus | aureus | sobrinus | stains |
| Producer | (% w/w) | T29 | 71-698 | OMZ175 | D39 | ATCC 15305 | A222 | OMZ176 | inhibited |
| K12 | 0.5% | 14 | 4.5 | 10 | 8 | 0 | 12 | nr | 5/6 |
| | 1.25% | 15 | 3.5 | 10 | 3 | 0 | 8 | 0 | 5/7 |
| | 1.7% | 12 | 3.5 | 0 | 2 | 0 | 9 | 0 | 4/7 |
| | 2.5% | 13 | 5.5 | 0 | 6 | 0 | 6 | 0 | 4/7 |
| | 3.3% | 10 | 1.5 | 0 | 0 | 0 | 0 | 0 | 2/7 |
| | 5% | 0 | 0 | 0 | 0 | 0 | 0 | 0 | 0/7 |
| M18 | 0.5% | 0 | 19.5 | 16 | 8 | 5 | 10 | 0 | 5/7 |
| | 1.25% | 8 | 13.5 | 0 | 5 | 0 | 6 | 0 | 4/7 |
| | 1.7% | 0 | 9.5 | 0 | 0 | 0 | 0 | 0 | 1/7 |
| | 2.5% | 16 | 16.5 | 0 | 6 | 0 | 0 | 0 | 2/7 |
| | 3.3% | 22 | 9.5 | 6 | 2 | 0 | 6 | 0 | 5/7 |
| | 5% | 17 | 9.5 | 0 | 0 | 0 | 0 | 0 | 2/7 |

nr = no result due to contamination

### Example 7C: Effect of Concentration of a Combination of Raffinose and Galactose to Induce Inhibitory Activity of K12 or M18

This experiment aimed to determine the effect of concentration of a combination of raffinose and galactose to induce the inhibitory activity of K12 and M18.

l Method: As above for Example 7B, except the CABCa agar plates were prepared with or without the following combinations of raffinose and galactose:

| Combination | Percentage of | Percentage of |
|---|---|---|
| No. | Raffinose | Galactose |
| 1 | 0.5% | 0.5% |
| 2 | 0.5% | 2.5% |
| 3 | 1.25% | 1.25% |
| 4 | 1.25% | 1.7% |
| 5 | 1.25% | 2.5% |
| 6 | 1.7% | 1.25% |
| 7 | 1.7% | 1.7% |
| 8 | 2.5% | 0.5% |
| 9 | 2.5% | 1.25% |
| 10 | 2.5% | 2.5% |

Results: As shown in Table 9, activity against a range of pathogens was observed for various combinations of weight percentage raffinose and galactose.

TABLE 9
Effect of different combinations of raffinose and galactose on the inhibitory activity of K12 and M18.

| | | | Size of zone inhibition normalised to control (mm) | | | | | | | |
|---|---|---|---|---|---|---|---|---|---|---|
| | | | S. | S. | S. | S. | S. | S. | S. | Total |
| | Raffinose | Galactose | constellatus | pyogenes | mutans | pneumoniae | saprophyticus | aureus | sobrinus | strains |
| Producer | (w/w) | (w/w) | T29 | 71-698 | OMZ175 | D39 | ATCC 15305 | A222 | OMZ176 | inhibited |
| K12 | 0.5% | 0.5% | 18 | 8.5 | 0 | 0 | 0 | 9 | nr | 3/6 |
| | 0.5% | 2.5% | 6 | 0 | 0 | 0 | 0 | 0 | 0 | 1/7 |
| | 1.25% | 1.25% | 18 | 0 | 0 | 4 | 0 | 9 | 0 | 3/7 |
| | 1.25% | 1.7% | 18 | 4.5 | 0 | 8 | 0 | 0 | 0 | 3/7 |
| | 1.25% | 2.5% | 13 | 12.5 | 0 | 15 | 0 | 0 | 0 | 3/7 |
| | 1.7% | 1.25% | 13 | 46.5 | 0 | 0 | 0 | 10 | 0 | 3/7 |
| | 1.7% | 1.7% | 10 | 5.5 | 0 | 2 | 0 | 0 | 0 | 3/7 |
| | 2.5% | 0.5% | 18 | 4.5 | 8 | 6 | 4 | 13 | 0 | 6/7 |
| | 2.5% | 1.25% | 8 | 1.5 | 0 | 0 | 0 | 0 | 0 | 2/7 |
| | 2.5% | 2.5% | 19 | 5.5 | 0 | 0 | 0 | 0 | 0 | 2/7 |
| M18 | 0.5% | 0.5% | 0 | 25.5 | 0 | 12 | 3 | 0 | 0 | 3/7 |
| | 0.5% | 2.5% | 0 | 7.5 | 0 | 0 | 0 | 0 | 0 | 1/7 |
| | 1.25% | 1.25% | 0 | 12.5 | 0 | 0 | 0 | 0 | 0 | 1/7 |
| | 1.25% | 1.7% | 0 | 5.5 | 0 | 0 | 0 | 0 | 0 | 1/7 |
| | 1.25% | 2.5% | 0 | 5.5 | 0 | 0 | 0 | 0 | 0 | 1/7 |
| | 1.7% | 1.25% | 0 | 13.5 | 0 | 18 | 0 | 0 | 0 | 2/7 |
| | 1.7% | 1.7% | 0 | 3.5 | 0 | 0 | 0 | 0 | 0 | 1/7 |
| | 2.5% | 0.5% | 0 | 9.5 | 0 | 4 | 0 | 0 | 0 | 2/7 |
| | 2.5% | 1.25% | 0 | 8.5 | 0 | 6 | 0 | 0 | 0 | 2/7 |
| | 2.5% | 2.5% | 0 | 6.5 | 0 | 0 | 0 | 0 | 0 | 1/7 |

### Example 8: Activity of Raffinose (2.5%) with K12 or M18 Compared Trimix (Mixture of Equal Weight Percentage Concentrations of the Three Saccharides) and Individual Saccharides

This experiment shows that the inhibitory spectrum of K12 or M18 was found to be extended to species and strains, not typically inhibited by K12 or M18. This concentration of raffinose was found to inhibit several ENTR, dental and skin pathogens and S. salivarius strains

The method used was the same as Example 4.

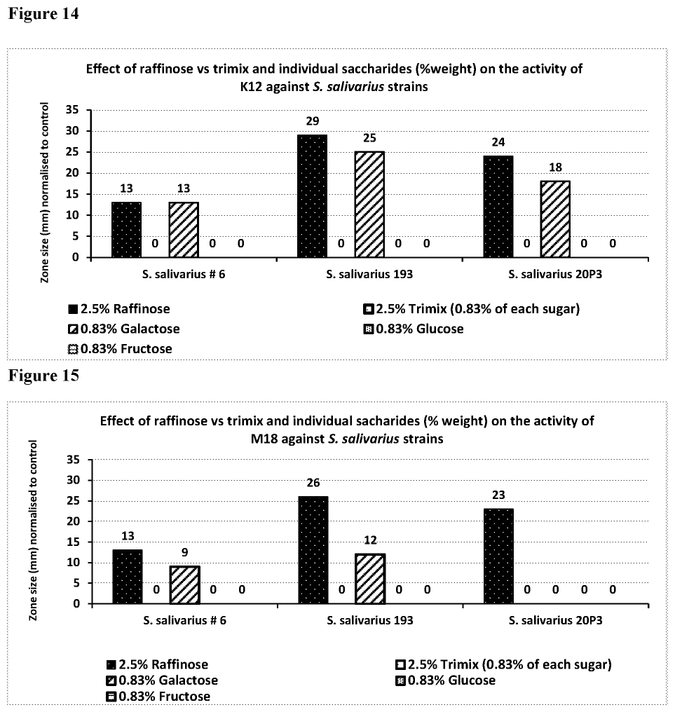

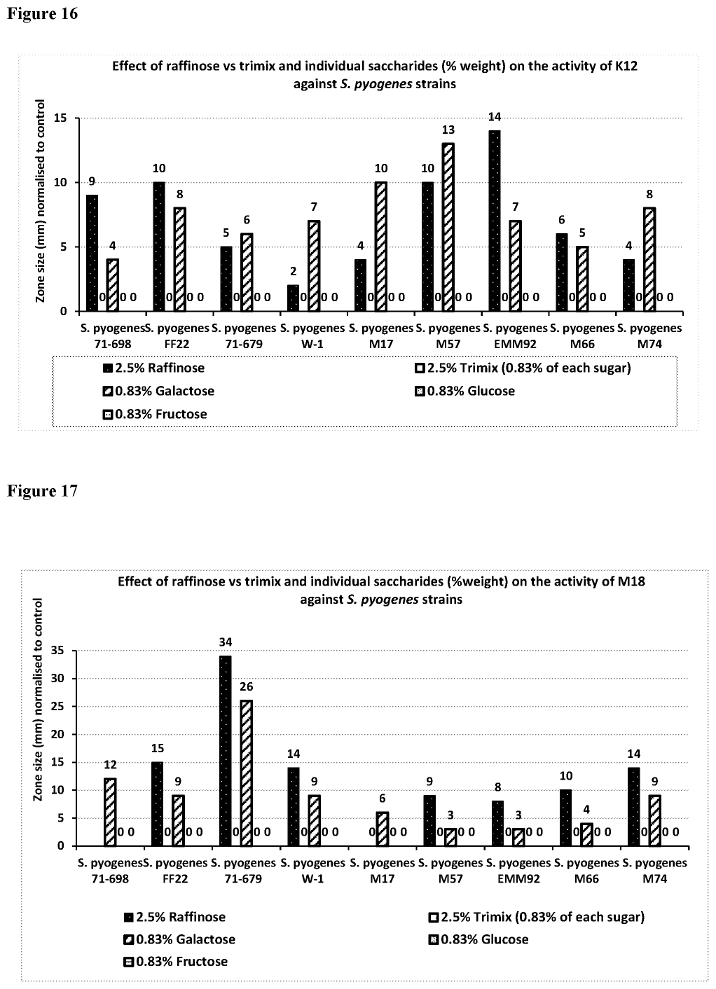

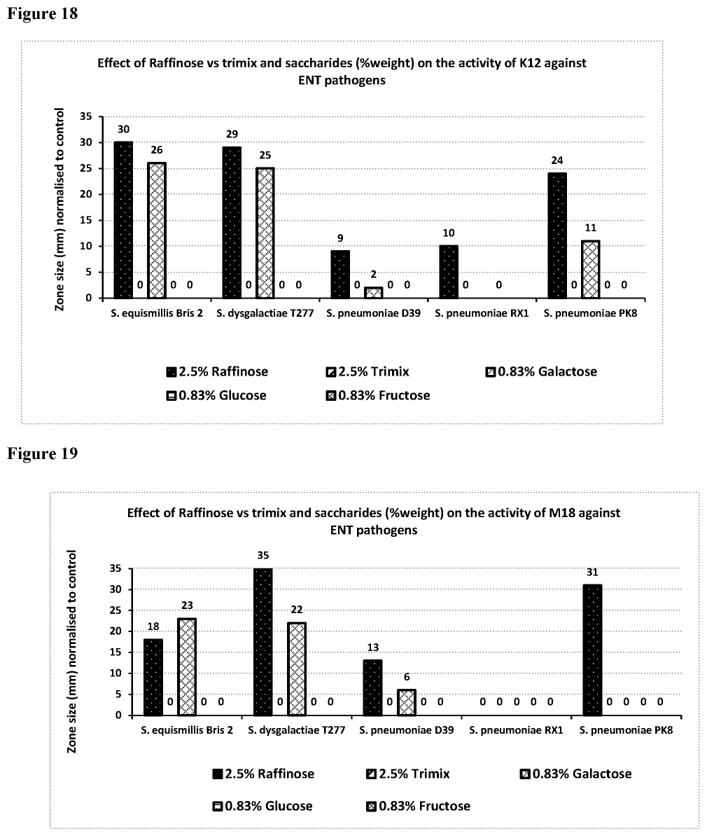

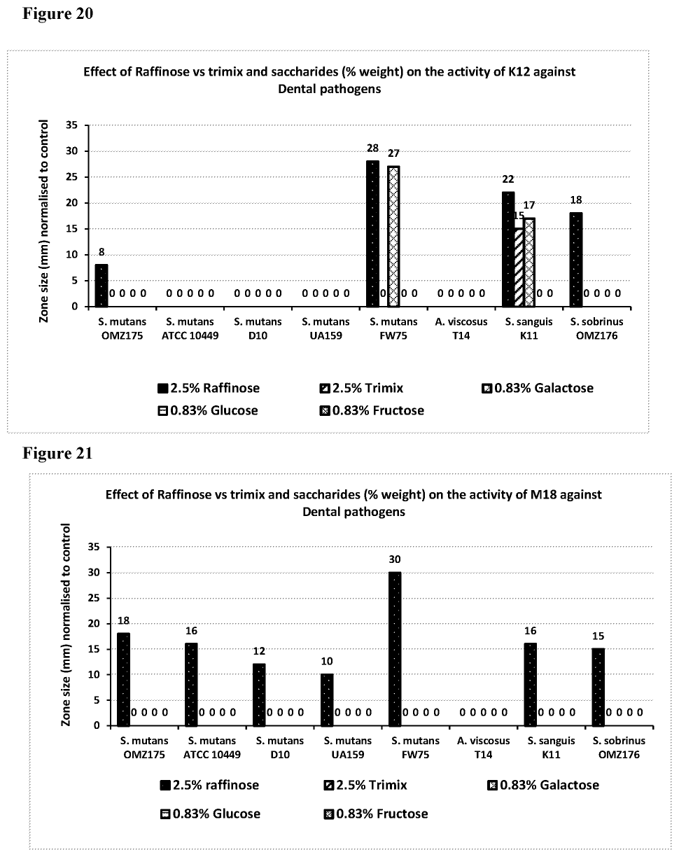

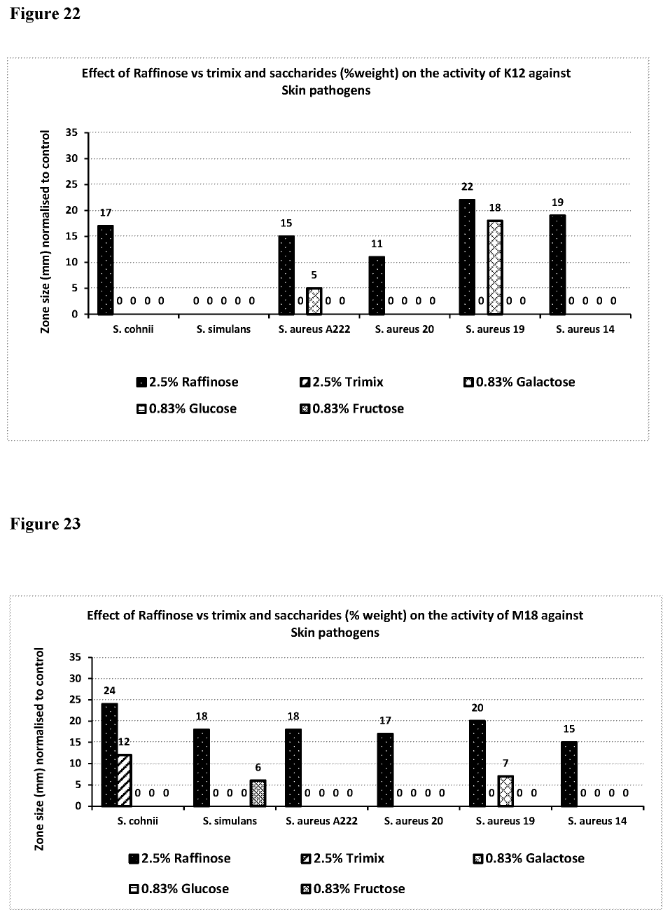

Results FIGS. 14-23 shows the inhibitory effects of K12 or M18 in against ENTR, dental and skin pathogens and other S. salivarius strains sensitive to bacteriocin producing S. salivarius K12 or M18.

### Example 9: Raffinose Promotes Inhibitory Activity of K12 and M18 when K12 and M18 is Sourced from a Different Fermentation Process and Contains a Different Lyoprotectant Mix i.e. Dairy Free

This experiment shows that raffinose enhances the inhibitory effect of K12 and M18 irrespective of the source of K12 in ingredient supplier and the different lyoprotectant matrix that houses K12 or M18.

The method used is described in example 4. The pH of the producer streak was adjusted after the growth of producer to change the acidic conditions for all test plates.

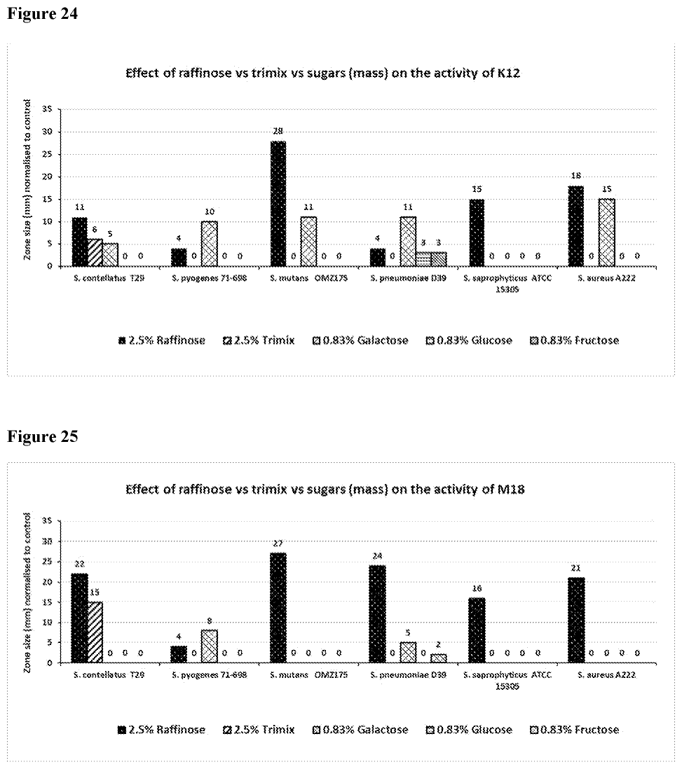

The enhanced inhibitory action of dairy free K12 with 2.5% w/w raffinose over an equal weight percentage of saccharides either as a trimix or individual saccharides was seen against representative microorganisms from skin, dental, oral, ENTR diseases and as well as other S. salivarius strains (FIG. 24-25). The activity is normalised to baseline control.

Conclusion: Similar to K12 or M18, dairy free K12 or M18 also showed enhanced inhibitory activity against a variety of microorganisms in presence of raffinose and galactose. Comparatively, trimix or other saccharides did not show similar inhibitory effect.

### Example 10A: Effect of Raffinose and Galactose on the Growth and Induction of Inhibitory Activity of K12

This example shows that raffinose and galactose do not contribute to greater cell count of K12 but still enhance antimicrobial effect.

Method: Growth curves were conducted using K12 raw ingredient as a starting culture of 1×10⁵ CFU/ml in M17 broth (control) [BD Difco #218561] or M17 broth (test) supplemented with either 2.5% raffinose [D-(+)-raffinose pentahydrate -Sigma #R0250], 2.5% Trimix, 0.83% or 2.5% galactose [D-galactose—BD Difco #216310], 0.83% or 2.5% glucose [D(+)-Glucose—monohydrate—Applichem #A3617] or 0.83% or 2.5% fructose [D(−)Fructose—Sigma #F3510](all w/v). Broth cultures were then incubated at 37° C., 5% CO₂ in air and samples were taken and analysed at following timepoints of 0, 6 and 18 hours for cell count and optical density using a spectrophotometer (Optical Density (OD) of 600 nm). To determine cell count, 1:10 serial dilutions of the culture samples were prepared in Phosphate Buffered Saline (PBS Dulbecco A—Oxoid #BR0014G) and then 20 μl spots of each dilution were spotted in triplicate onto CABK12 agar plates. These were then incubated at 37° C., 5% CO₂ in air for 18 h. The cell count for each culture sample at the different timepoints was then calculated from the number of colonies grown at each of the 1:10 dilutions.

### Results:

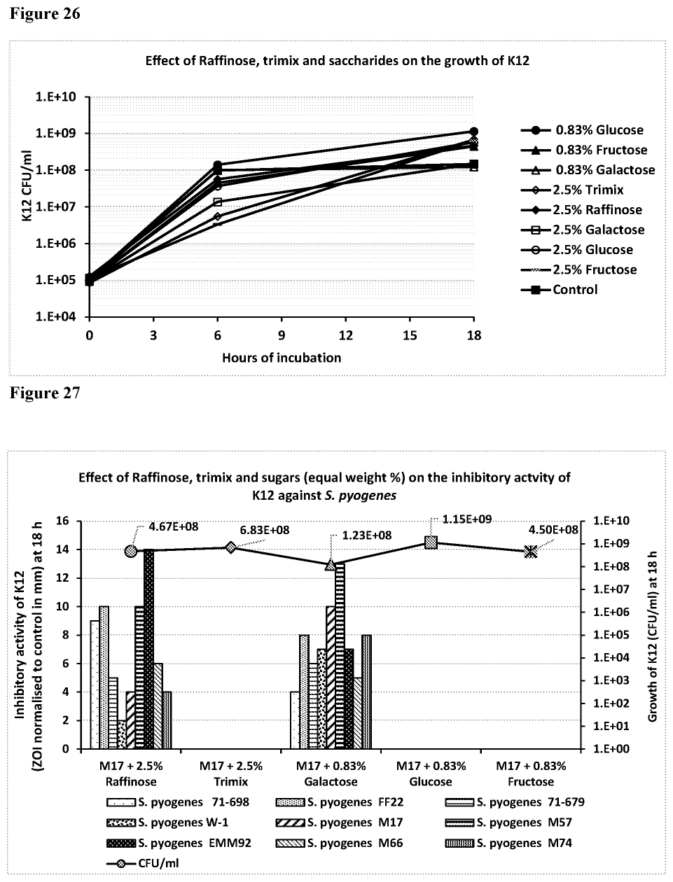

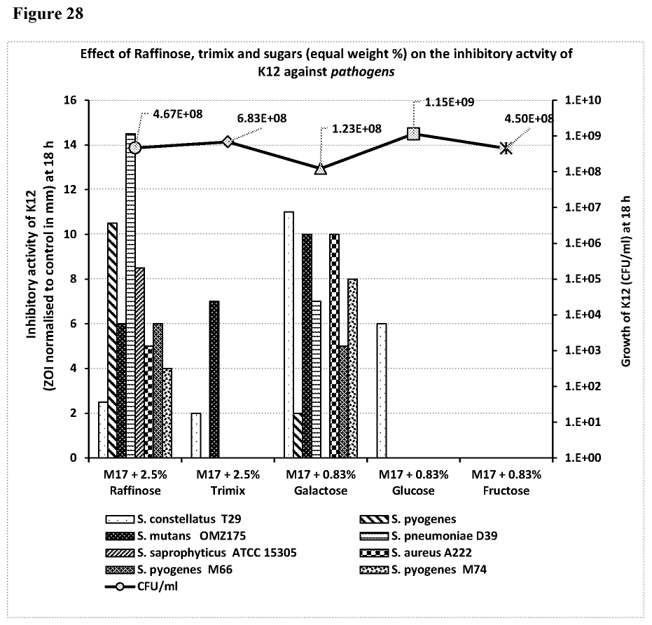

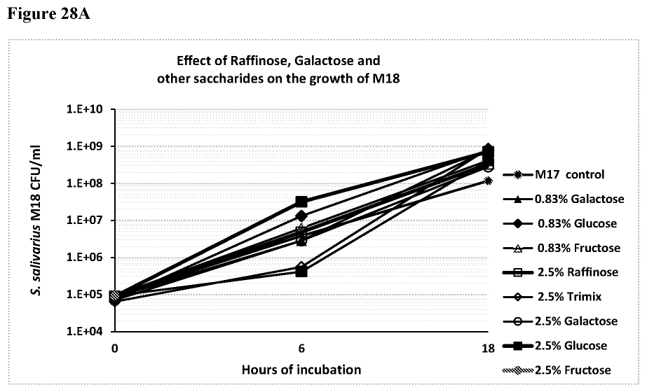

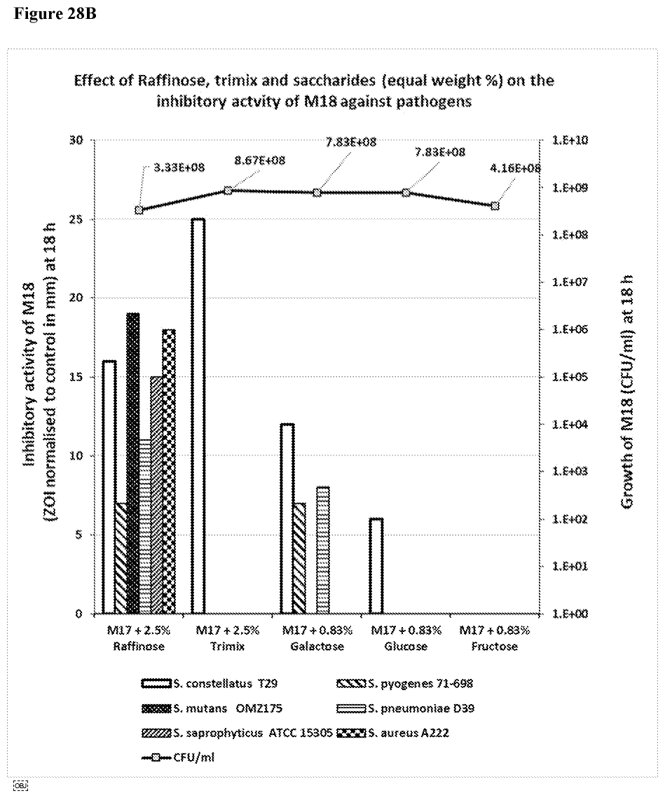

All saccharide combinations resulted in a similar cell counts for K12, thus they did not result in any substantial difference except for glucose which gave the highest cell count (FIG. 26).
But despite this, glucose did not result in any greater inhibitory activity (likely due to catabolite repression).
Surprisingly K12 grown in presence of raffinose and galactose shows greater inhibitory activity against S. pyogenes strains (FIG. 27) or other pathogens (FIG. 28).
Trimix or individual saccharides of raffinose as supplied separately did not result in any greater increase in inhibitory activity compared to the control.

### Example 10B: Effect of Raffinose and Galactose on the Growth and Induction of Inhibitory Activity of M18

This example shows that raffinose and galactose do not contribute to greater cell count of S. salivarius M18 but still enhance antimicrobial effect.

Method: Growth curves were conducted using M18 raw ingredient as a starting culture of 1×10⁵ CFU/ml in M17 broth (control) [BD Difco #218561] or M17 broth (test) supplemented with either 2.5% raffinose [D-(+)-raffinose pentahydrate -Sigma #R0250], 2.5% Trimix, 0.83% or 2.5% galactose [D-galactose—BD Difco #216310], 0.83% or 2.5% glucose [D(+)-Glucose—monohydrate—Applichem #A3617] or 0.83% or 2.5% fructose [D(−)Fructose—Sigma #F3510](all w/v). Broth cultures were then incubated at 37° C. in 5% CO₂ and samples were taken and analysed at following timepoints of 0, 6 and 18 hours for cell count. To determine cell count, 1:10 serial dilutions of the culture samples were prepared in Phosphate Buffered Saline (PBS Dulbecco A—Oxoid #BR0014G) and then 20 μl spots of each dilution were spotted in triplicate onto CABK12 agar plates. These were then incubated at 37° C. with 5% CO₂ for 18 h. The cell count for each culture sample at the different timepoints was then calculated from the number of colonies grown at each of the 1:10 dilutions.

### Results:

All saccharide combinations resulted in a similar cell counts for M18 (FIG. 28A).
Surprisingly M18 grown in presence of raffinose, and galactose shows greater inhibitory activity against S. pyogenes strain as well as other microorganisms (FIG. 28B).
Trimix or individual saccharides of raffinose as supplied separately did not result in any greater increase in inhibitory activity compared to the control.

### Example 11: Cell Morphology of K12 and M18 Raw Ingredient Product Grown Either with and without Raffinose or a Trimix of Saccharides Galactose, Glucose or Fructose, or the Individual Saccharides

The aim of this experiment was to determine whether S. salivarius K12 and M18 raw ingredient (−) produces sticky mucoid like colonies (compared to control). when grown on either CABCa control plates or CABCa test plates supplemented with 2.5% raffinose or 2.5% trimix (0.83% of each of saccharides: galactose, glucose and fructose) or 0.83% of the individual saccharides.

S. salivarius K12 and M18 sourced from dairy free raw ingredient was also analysed for morphology changes when grown on either CABCa control plates or CABCa test plates supplemented with 2.5% raffinose or 2.5% trimix (0.83% of each of saccharides: galactose, glucose and fructose).

Method: Columbia Blood Agar+0.5% CaCO₃ (CABCa—To a 180 ml bottle, 0.9 g of Calcium carbonate (CaCO₃) PanReac Applichem #141212,1210 was added to the bottle of agar by preparing a well in the agar using a scalpel and tipping in the calcium carbonate and covering with the cut-out agar. The agar was then melted in the autoclave for 110° C./10 mins before cooling and mixing well before pipetting 20 ml into petri dishes.

CABCa+raffinose (2.5% w/v)—To a 180 ml bottle, 0.9 g of CaCO₃ and 4.5 g (2.5%) of D-(+)-raffinose pentahydrate (Sigma #R0250) was added to the bottle of agar by preparing a well in the agar using a scalpel and tipping in the calcium carbonate and covering with the cut-out agar. The agar was then melted in the autoclave for 110° C./10 mins before cooling and mixing well before pipetting 20 ml into petri dishes.

CABCa+Trimix (mixture of equal weight percentage concentrations of the three saccharides).—To a 180 ml bottle, 0.9 g of CaCO₃ and 1.5 g (0.83%) of each saccharide: D-galactose, D(+)-Glucose and D(−)Fructose was added to the bottle of agar by preparing a well in the agar using a scalpel and tipping in the calcium carbonate and carbohydrate and covering with the cut-out agar. The agar was then melted in the autoclave for 110° C./10 mins before cooling and mixing well before pipetting 20 ml into petri dishes.
D-(+)-raffinose pentahydrate—Sigma #R0250 mol. wt=594.51 g/mol
D-galactose—BD Difco #216310 mol wt=180.16 g/mol
D(+)-Glucose—monohydrate—Applichem #A3617,1000 mol.wt=198.17 g/mol
D(−)Fructose—Sigma—#F3510 mol.wt=180.16 g/mol

CABCa agar test plates were prepared, supplemented with either the individual saccharides; galactose, glucose and fructose or a trimix of the saccharides at a concentration of 0.83% w/v. Also, CABCa test plates supplemented with raffinose at 2.5% w/v were prepared. All test plates were supplemented with 0.5% calcium carbonate. Also, CABCa agar supplemented with 0.5% w/v calcium carbonate plates were prepared as control plates.

Freeze dried raw ingredient:—K12 -1.94×10¹¹ cfu/g, -dairy free K12 -2.4×10¹¹ cfu/g, M18 -2.2×10¹¹ cfu/g, dairy free M18 -4.2×10¹¹ cfu/g.

Diluent: Todd Hewitt Broth (THB)—30 g Todd Hewitt Broth Powder (BD Difco #279240)+Distilled Water (1000 ml) autoclaved for 121° C./15 mins.

Producer preparation: 1 g of raw ingredient was added to 9 ml THB in a small stomacher bag giving a 1:10 dilution approx. 1×10¹⁰ cfu/ml. This was then mixed in the stomacher machine for 5 mins. Then 1:10 serial dilutions were carried out in THB down to 10⁻⁴. cfu/ml.

Spread plate: 20 μl volumes of the 10⁻⁴ cfu/ml dilution of raw ingredient were spread onto either CABCa control or CABCa test plates. These were then incubated for 18 hours at 37° C. with 5% CO₂. The plates were then visualised and photographed to assess morphology changes.

Deferred assay plate: These were prepared as before by dispensing 100 μl of the 10⁻³ dilution of raw ingredient as droplets in a vertical line down the middle of the test CABCa plates supplemented with or without saccharides and then spread as 1.2 cm streak down the middle of the plate using a cutdown plastic spreader. Plates were incubated for 18 h at 37° C. with 5% CO₂.

The raw ingredient producer streak was photographed then removed using a glass slide to visualise the bacterial mass and morphology.

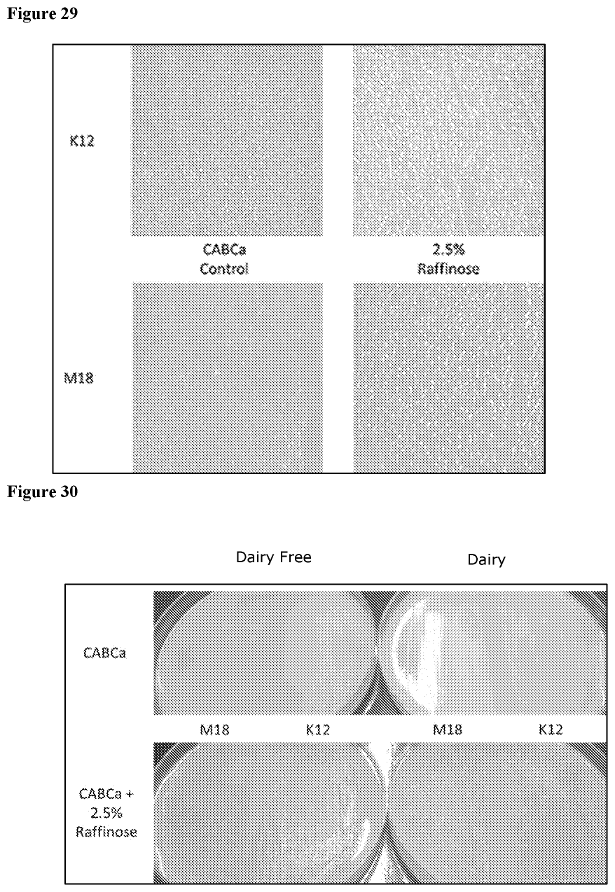

Results: Spread plates: When S. salivarius K12 and M18 are grown on 2.5% w/v raffinose, larger mucoid colonies are seen than when grown on CABCA control plates. K12 and M18 colonies on CABCa test plates supplemented with either Trimix (mixture of equal weight percentage concentrations of the three saccharides) or individual saccharides, were not mucoid looking (FIG. 29).

Magnified photos of K12 and M18 colonies on CABCa agar and CABCa agar supplemented with 2.5% w/v raffinose: Compared to control, S. salivarius K12 or M18 (dairy or dairy free) visually appear as large sticky mucoid like colonies. This effect was not observed with or 2.5% w/v trimix (0.83% w/v mixture of equal weight percentage concentrations of the three saccharides) and or 0.83% w/v of the individual saccharides (FIG. 30).

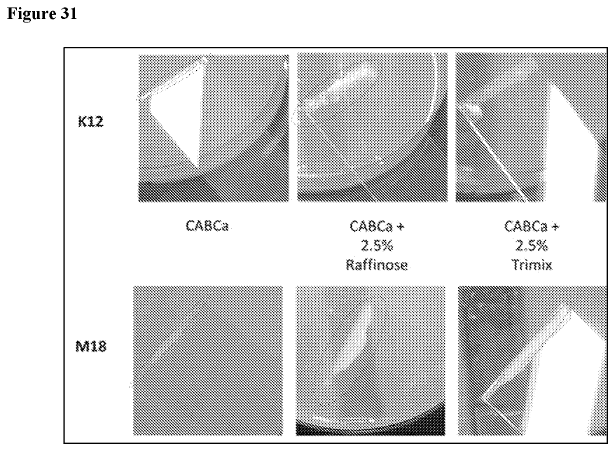

Photos of K12 producer streaks and glass slides showing bacterial growth. Dotted black ovals visually shows amount of mucous produced (FIG. 31).

Photos of M18 producer streaks and glass slides showing bacterial growth. Dotted black ovals visually shows amount of mucous produced (FIG. 31).

K12 and M18 producer streaks also look very mucoid when they are grown on CABCa supplemented with 2.5% w/v raffinose (FIG. 31).

### Example 12: Induction of Antimicrobial Effect (Inhibition) Varies by S. salivarius Strain

Aim: to determine whether the induction of antimicrobial effect in the presence of supplemental saccharides is an inherent property of all S. salivarius strains.

Method: Preparation of solid culture media—CABCa agar plates were prepared with or without either 2.5% w/w raffinose or 0.5% w/w galactose by the addition of 0.5% (w/v) calcium carbonate to solid CAB agar in bottles and then melted by autoclaving at 110° C. for 10 mins. Once cooled, filter sterilised solutions of the saccharides or distilled water were added and mixed and then 20 ml pipetted into petri dishes.

Preparing S. salivarius producer suspension, about 6 colonies of each S. salivarius strain were added into separate tubes containing 1 ml THB and mixed well.

Deferred antagonism assay—100 μl of S. salivarius producer suspensions was dispensed onto CABCa plates supplemented with or without raffinose or galactose as droplets in a vertical line down the middle of the test plate. This suspension was then spread as 1.2 cm streak down the middle of the plate using a cutdown plastic spreader. Plates were incubated for 18 hours at 37° C., 5% CO₂ in air.

After incubation, the bacterial growth was then removed from the agar plate using a sterile cotton swab. The pH of the producer streak was then adjusted by placing a 1 cm wide strip of filter paper soaked in 0.5M sodium carbonate pH 11 onto the agar plate to buffer the acid and adjust the pH up to around pH 6.5-7.5.

Plates were surface sterilised with chloroform vapour for 30 minutes, followed by air drying for 30 minutes.

Bacterial indicator suspensions were prepared by adding 3-9 colonies (depending on the size) of each strain into separate tubes containing 3 ml THB. These suspensions were then swabbed across the agar plate perpendicular to the producer streak and then the agar plate was incubated for a further 18 hours at 37° C., 5% CO₂ in air.

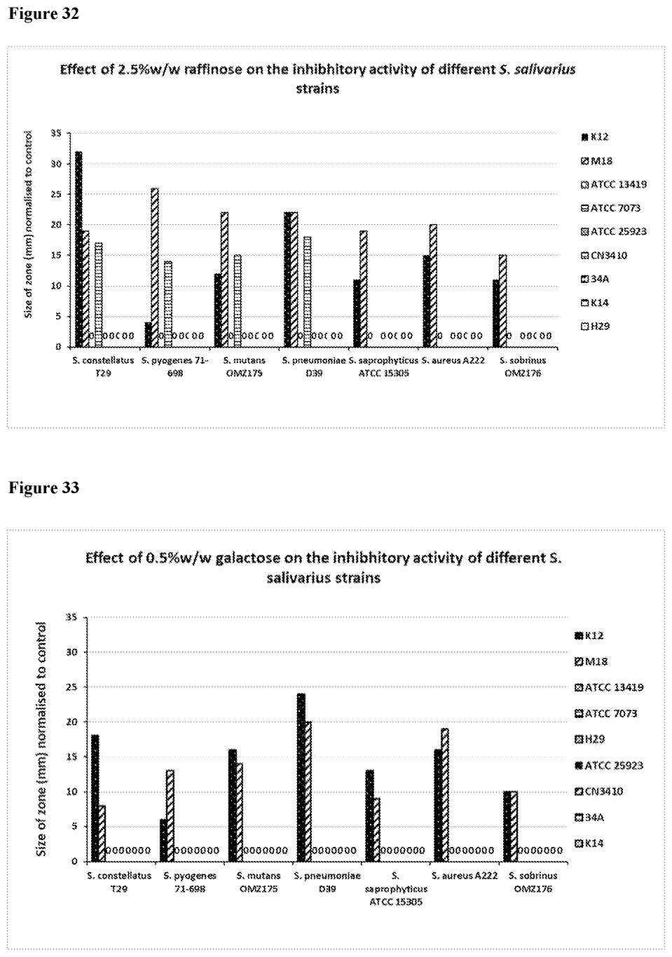

Results: A range of S. salivarius strains were assayed including strains obtained from ATCC. When grown in presence of a specific concentration of 2.5% w/w of raffinose (FIG. 32) or 0.5% w/w of galactose (FIG. 33), almost all bar one strain did not show inhibitory activity against pathogens implicated in the ENT and Skin infections. The exception was S. salivarius strain ATCC 7073, which was the only other strain apart from K12 and M18 to have antimicrobial activity when supplemented with 2.5% w/w raffinose.

Conclusion: Induction of inhibitory activity in S. salivarius by raffinose or galactose is not an inherent property of S. salivarius. Supplementing other S. salivarius strains with galactose does not induce the same inhibitory effect as was observed for K12 and M18. With the exception of S. salivarius strain ATCC 7073, supplementing other S. salivarius strains with raffinose does not induce same inhibitory effect as was observed for K12 and M18.

### Example 13: Induction of Antimicrobial Activity in K12 or M18 Against Gram-Negative Pathogens, Including Those Implicated in Causing Halitosis

The aim of this experiment was to investigate the induction of inhibitory activity by galactose or raffinose in K12 or M18 against gram-negative bacteria F. nucleatum, P. gingivalis and P. intermedia which are not inhibited by K12 or M18 alone, and can cause halitosis and other dental infections.

Method: CABCa agar plates were prepared with or without 2.5% w/w raffinose, 0.5% w/w galactose or a combination of both saccharides by the addition of 0.5% (w/v) calcium carbonate to solid CAB agar in bottles and then melted by autoclaving at 110° C. for 10 mins. Once cooled, filter sterilised solutions of the saccharides or distilled water were added and mixed and then 20 ml pipetted into petri dishes.

A deferred antagonism assay was conducted using K12 or M18 raw ingredient suspended in THB. This was spread as a 1.2 cm streak containing approximately 1-2×10⁶ cfu down the middle of a CABCa control or CABCa galactose supplemented test plate. After 18 hrs growth at 37° C., 5% CO₂ in air, bacterial growth was removed, and the pH of the agar in the initial streak was measured and adjusted to a pH of 6.5-7.5 using 0.5M sodium carbonate pH11 before sterilizing the agar surface using chloroform vapor. Bacterial test strains were suspended in THB and swabbed across the plates perpendicular to the initial streak and then the plates were incubated at 37° C. in an anaerobic jar containing an anaeroGen sachet for 4 days. Zones of inhibition for each test strain were then measured (mm).

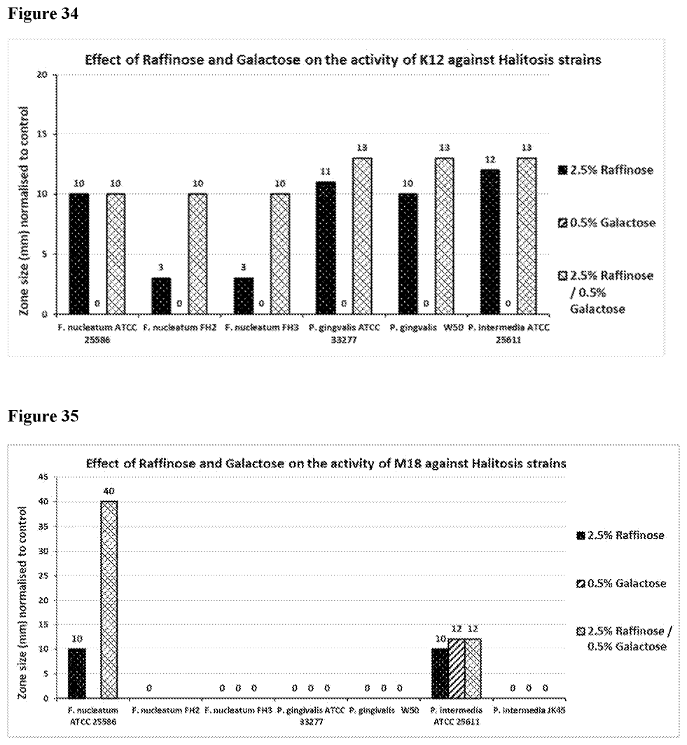

Result: K12 was found to inhibit a variety of strains of halitosis-causing gram-negative bacteria when supplemented with either 2.5% w/w raffinose or a combination of 2.5% w/w raffinose and 0.5% w/w galactose (FIG. 34). A solution of 0.5% w/w galactose on its own did not induce antimicrobial activity in K12 against any of the bacterial species associated with halitosis tested.

M18 was also found to inhibit some strains of halitosis-causing bacteria when supplemented with either 2.5% w/w raffinose or a combination of 2.5% w/w raffinose and 0.5% w/w galactose (FIG. 35).

### Example 14: Inhibitory Effect when Galactose and Raffinose are Supplemented to K12 and M18 Freeze Dried Raw Ingredient Powder

Aim: To determine the inhibitory activity of K12 and/or M18 raw ingredient powder in presence of raffinose and/or galactose.

Method: The amount of raffinose and galactose used was calculated based on the volume of the area of the producer streak, (which was calculated to be 4.25 g) to allow for the absorption of the saccharides into the agar. Based on this, amount of 0.021 g (i.e. 0.5% w/w galactose of 4.25 g agar volume) of galactose, 0.11 g (i.e 2.5% w/w) of raffinose and the combination of galactose (0.5% w/w) and raffinose (2.5% w/w) (0.13 g total) were weighed into sterile containers. K12 and/or M18 raw ingredient were suspended and diluted with sterile distilled water to a concentration of 1-2×10⁶ cfu/100 μl. Then, 100p of K12 only, M18 only, and K12±M18 suspensions were spread as a 1 cm streak down the middle of a CABCa agar plate using a sterile spreader. In addition, 100 μl suspensions of K12 only, M18 only and K12±M18 were mixed using a sterile stirring rod with the galactose and/or raffinose powders weighed above. The total volume of each mixture was pipetted using a large bore tip down the centre of a CABCa agar plate and spread as a 1 cm streak down the middle with a sterile spreader. All plates were incubated lid upwards for 18 h at 37° C., 5% CO₂ in air. Bacterial growth was then removed using a microscope slide, and the pH of the agar in the producer streak area was measured and adjusted to pH 6.5-7.5 using 0.5M sodium carbonate (pH 11) before surface sterilizing the plates with chloroform vapour. Indicator bacterial test strains were suspended in 3 ml sterile THB and swabbed across the plates perpendicular to the producer steak area. The plates were incubated for a further 18-24 h at 37° C., 5% CO₂ in air. Zones of inhibition for each test organism were then measured in mm using a ruler. The amount of raffinose and galactose to be used was calculated based on the volume of the area of the producer streak, (which was calculated to be 4.25 g) to allow for absorption into the agar.

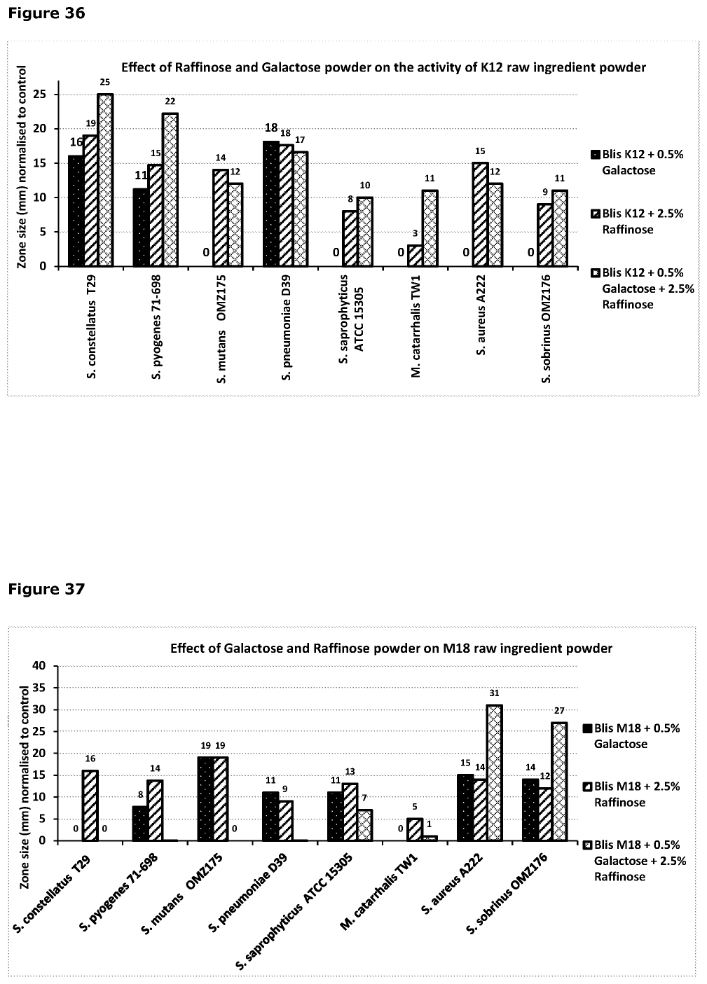

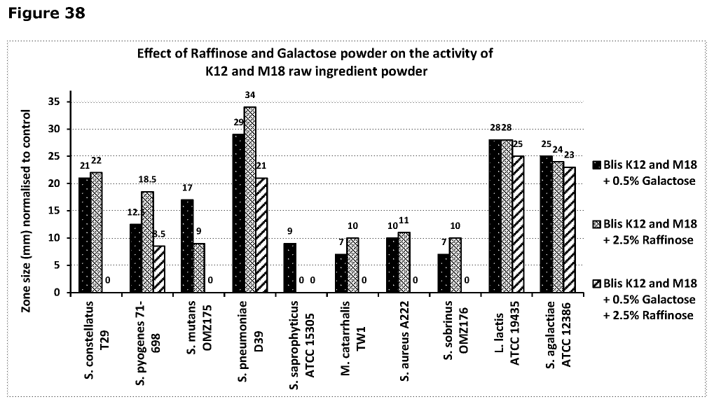

Results: Both galactose (0.5% w/w) and raffinose (2.5% w/w) and their combinations were found to have induced the inhibitory activity in the freeze-dried raw ingredient powder of K12 (FIG. 36) or M18 (FIG. 37) or K12 and M18 combination (FIG. 38).

### Example 15: Effect of Raffinose and Galactose on the Induction of Inhibitory Activity in a Commercial Powder Formulation (Daily Defence Junior) Containing S. salivarius K12

Aim: To compare the inhibitory effect of S. salivarius K12 in a commercial powder formulation Daily Defence Junior (Composition: S. salivarius K12 (1.25×10⁹ cfu/0.8 g), Isomalt, Maltodextrin, Vanilla flavour) with the supplemental saccharides raffinose and galactose added.

Method: Formulations for testing were prepared as follows:
1. Control: Commercial powder formulation (Daily Defence Junior) containing S. salivarius K12
2. Galactose 0.5% w/w was added to the commercial powder and mixed thoroughly to achieve a uniform mixture.
3. Raffinose 2.5% w/w was added to the commercial powder and mixed thoroughly to achieve a uniform mixture.

The following method was used to measure the inhibitory effect of S. salivarius K12 in the context of the powder formulations. Exactly 40 mL of 50° C. molten CAB agar containing 0.5% calcium carbonate (CABCa) was poured in agar plates (120×120 mm). Once the agar was set upon cooling, it was split in the middle and half of the agar gel was removed. The other half was left on the plate as Blank agar marked as side “A”. Approximately, 0.8 g of each combination (DDJ powder with (1) galactose, (2) raffinose or (3) combination of raffinose and galactose) was mixed with 1 mL of sterile distilled water and vortexed to produce a homogeneous suspension. 100 μl of the suspension was reserved for spreading on the surface of agar (producer side B). The remaining suspension was then mixed with 20 mL of molten CABCa agar and the mixture was poured into the empty half of the agar plate to form producer side “B”. The 100 μL of the reserved suspension was spread over the surface on side B and the plates were incubated for 18 h, 37° C., 5% CO₂ in air. Post incubation, bacterial growth on the producer side B was removed. The pH of the producer side was measured and adjusted to 6.5-7.5 by soaking into the agar a solution of 0.5 M sodium carbonate pH 11. The plate surface was sterilized with chloroform vapours. Bacterial indicator strains were suspended in THB and were streaked across the agar plates from left (blank agar side A) to right (producer side B) using sterilised cotton swabs. Plates were again incubated for 18 h at 37° C., 5% CO₂ in air. The zone of inhibition of each indicator strain was measured (in mm) using a ruler. Results were also read using a macroscope to establish presence of small colonies or complete inhibition zones.

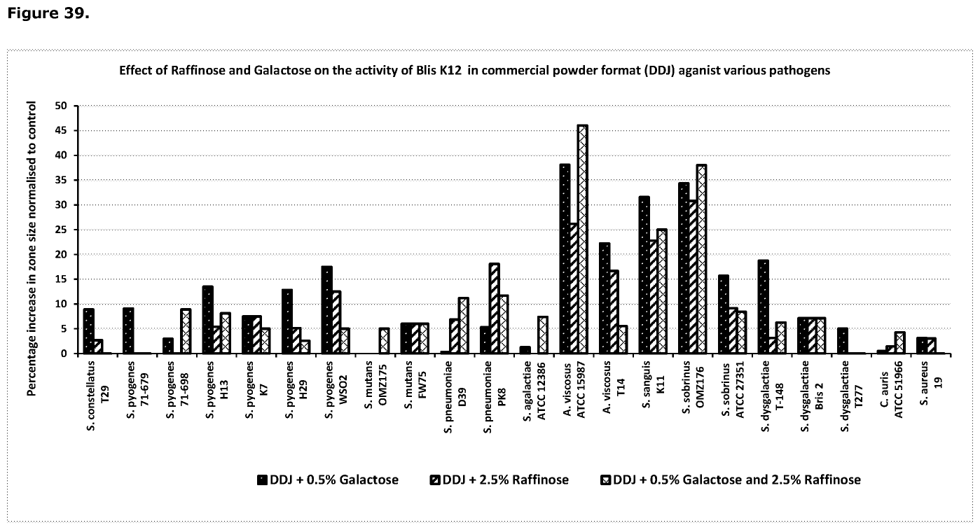

Results: FIG. 39 shows that the inhibitory activity of S. salivarius K12 was increased in the powders containing raffinose (2.5% w/w), galactose (0.5% w/w) or their combination (raffinose 2.5% w/w+galactose 0.5% w/w) compared to the commercial powder containing K12 on its own (control).

### Example 16: Properties of Formulation from US 20190343899 A1

Aim: To determine the manufacturing conditions for the prior art formulation from US published patent application 20190343899.

Method: Formulation was prepared following the directions in example 1 of prior art D1. Briefly, liquid ingredients were mixed together and added slowly to the solid ingredients and heated to around 100° C. on a hotplate until melted. The mixture was then allowed to cool down until it completely solidified at approximately 60° C.

Results: The formulation described in Example 1 of US 20190343899 was prepared by melting the ingredients to prepare a formulation having the consistency of hard candy or toffee. A high temperature (around 100° C.) was required to melt the ingredients. Due to high heat, S. salivarius could not be added to the formulation, as temperature above 50° C. is detrimental to the probiotic. For this reason it was not possible to add a probiotic to the formulation at the melt stage (above approximately 60° C.), particularly a heat-sensitive probiotic such as S. salivarius, without total loss of probiotic.

During cooling, the consistency of the formulation which would have enabled admixture of probiotics into the formulation with sufficient homogeneity was only maintained at a temperature of 60° C. or higher. This temperature was still too high to add the probiotic without causing cell death which occurs from around 50° C. or higher.

### Example 17: Comparative Example—Formulation from WO 2017129639 Al

Method: A powdered infant nutrition product (infant formula) of similar composition to that of Example 1 of the publication WO 2017129639 was purchased (Similac 360 Total Care (Abbott Global). This product contains vitamins, minerals, lactose, 5 human oligosaccharides and whole milk powder.

To determine the effect of supplemental saccharides on the induction of inhibitory activity in S. salivarius K12 in the infant formula, S. salivarius K12 and supplemental saccharides were added to the infant formula as follows.
1. Control: S. salivarius K12 was added to the infant formula powder and mixed thoroughly to achieve a uniform mixture containing approx. 1.25×10⁹ cfu/g S. salivarius K12.
2. Galactose 0.5% w/w: S. salivarius K12 was added to the infant formula powder and 0.5% w/w galactose and mixed thoroughly to achieve a uniform mixture containing approx. 1.25×10⁹ cfu/g S. salivarius K12.
3. Raffinose 2.5% w/w: S. salivarius K12 was added to the infant formula powder and 2.5% w/w raffinose and mixed thoroughly to achieve a uniform mixture containing approx. 1.25×10⁹ cfu/g S. salivarius K12.

Measurement of induction of inhibitory activity was conducted in the same way as for Example 15.

Results: Surprisingly, the addition of galactose or raffinose to the control formulation of Example 17 showed a reduction in inhibitory activity compared to the control (Table 10).

### Example 18: Comparison of Antimicrobial Properties of Powder Formulations

Method: The antimicrobial activity of a number of compositions was tested, as follows.
1. Control formulation of Example 17;
2. Whole milk Powder: S. salivarius K12 was added to the whole milk powder (Anchor Blue™ Milk powder, Anchor, NZ) and mixed thoroughly to achieve a uniform mixture containing approx. 1.25×10⁹ cfu/g S. salivarius K12

Measurement of induction of inhibitory activity of the powder formulations was conducted in the same way as for Example 15.

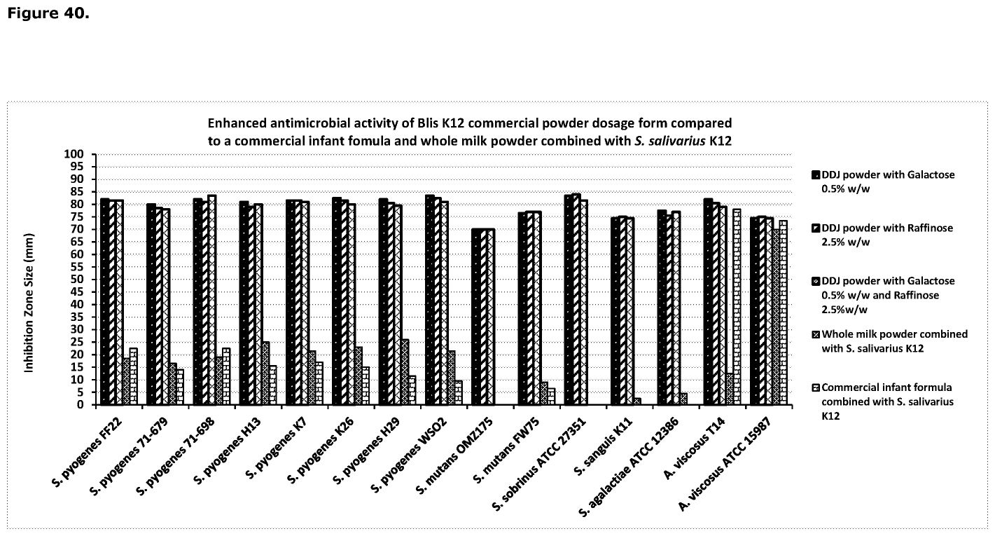

Results: Surprisingly these two formulations induced none, or less significant inhibitory activity compared with the three formulations of Example 15, containing commercial S. salivarius K12 Daily Defence Junior powder product which had been supplemented with raffinose, galactose and combination thereof (FIG. 40).

TABLE 10
Effect of Raffinose and Galactose on the inhibitory activity of S. salivarius K12 in an infant formula against various pathogens.

| | % change in zone size (in mm) normalised to control | | | | | | | | | |
|---|---|---|---|---|---|---|---|---|---|---|
| | S. | S. | S. | S. | S. | S. | S. | S. | S. | S. |
| | constellatus | pyogenes | mutans | mutans | pneumoniae | pneumoniae | aureus | aureus | cohnii | simulans |
| | T29 | 71-698 | FW75 | OMZ175 | D39 | PK8 | #19 | A222 | ATCC 29974 | ATCC 27848 |
| A | −12 | −3 | 13 | 0 | −4 | −46 | −60 | −60 | 100 | 300 |
| B | −20 | 0 | 0 | −8 | −4 | −35 | −100 | −100 | −100 | −100 |

| | % change in zone size (in mm) normalised to control | | | | | | | | | |
|---|---|---|---|---|---|---|---|---|---|---|
| | S. | A. | C. | | | | | | | |
| | agalactiae | viscosus | auris | S. | S. | S. | S. | S. | S. | |
| | ATCC | ATCC | ATCC | sanguis | sobrinus | sobrinus | dysgalactiae | dysgalactiae | dysgalactiae | S. |
| | 12386 | 15987 | 51966 | K11 | OMZ176 | ATCC 27351 | Bris 2 | T277 | T-148 | cohnii |
| A | 0 | 0 | −14 | −10 | 13 | 4 | 17 | 75 | 21 | −42 |
| B | 0 | −20 | −20 | −31 | −13 | −13 | −17 | −17 | −31 | −100 |

A = Infant formula Formulation 2 of Example 17 containing Galactose 0.5% w/w,
B = Infant formula Formulation 3 of Example 17 containing Raffinose 2.5% w/w,
− sign denotes reduction in activity compared to control Formulation 1 of Example 17

### Example 19: Upregulation of Beneficial Genes

Aim: To investigate upregulation of genes encoding antimicrobial molecules, and any other beneficial genes within the S. salivarius K12 genome which may cause the increased antimicrobial potency in the presence of galactose or raffinose.

Method: CABCa agar plates were prepared either with or without 2.5% w/w raffinose, 0.5% w/w galactose, combined 2.5% w/w raffinose and 0.5% w/w galactose, 0.5% w/w glucose or 2.5% w/w glucose by the addition of 0.5% (w/v) calcium carbonate to solid CAB agar in bottles and then melted by autoclaving at 110° C. for 10 mins. Once cooled, filter sterilised solutions of the saccharides or distilled water were added and mixed and then 20 ml pipetted into petri dishes.

A S. salivarius K12 raw ingredient suspension was prepared in PBS containing approx. 1×10⁸cfu/ml. 100p of this suspension was spread evenly onto control and test agar plates. Plates were incubated at 37° C. with 5% CO₂ for approx, 19 hrs.

Bacterial growth from each plate was collected using a sterile cotton swab and resuspended into 1 ml PBS in a screw capped tube. The bacterial cells were then pelleted by centrifugation at 13,000 rpm for 1 minute at 4° C. The supernatant was removed, and the cell pellet was resuspended in 1 ml of TRIzol reagent. The bacterial cell suspension was transferred into a 2 ml screw-capped tube containing 0.1 mm Zirconia/silicon beads. These tubes were then vortexed for 5 minutes at the highest speed to bead beat the bacterial cells to lyse them. The tubes were placed on ice for 1 minute and then re-bead beated again on the vortex for 5 minutes. The tubes were then frozen at −20° C.

The lysed bacterial suspension was thawed and 0.2 ml chloroform was added to each tube. The tubes were incubated for 2-3 minutes and kept on ice and also mixed by hand by inverting the tubes frequently. The beads were then left to settle, and then the upper liquid suspension was transferred into the phase-maker tubes. The tubes were incubated on ice for 5 minutes whilst mixing by hand by inverting the tubes frequently. The tubes were then centrifuged at 12,000 rpm at 4° C. for 15 minutes to separate into a lower (red) phenol-chloroform phase, interphase and upper (clear) aqueous phase.

560 μl of the clear upper aqueous phase was transferred into a new Eppendorf tube and then frozen again at −20° C.

The laboratory bench and other equipment used for the RNA extraction was treated with RNaseZap to remove any potential RNases.

RNA was extracted from the TRIzol thawed suspensions using the Thermofisher PureLink RNA mini kit following the manufactures' instructions for extracting RNA from TRIzol samples as follows:

Added 600 μl of 70% ethanol to each tube of thawed lysed bacterial suspension. Transferred approx. 600 μL of the sample to a spin cartridge within a Collection Tube.

Centrifuged at 12,000×g for 15 seconds at room temperature. Discarded the flow-through and reinserted the spin cartridge into the same collection tube. Added the final 600 μl of the sample and centrifuged the spin column again. Discarded the flow-through and reinserted the spin cartridge into the same collection tube.

Added 700 μL Wash Buffer I to the spin cartridge. Centrifuged at 12,000×g for 15 seconds at room temperature. Discarded the flow-through and the collection tube. Inserted the spin cartridge into a new collection tube.

Added 500 μL Wash Buffer II to the spin cartridge.

Centrifuged at 12,000×g for 15 seconds at room temperature. Discarded the flow-through and reinserted the spin cartridge into the same collection tube. Added another 500 μL Wash Buffer II to the spin cartridge and centrifuged at 12,000×g for 1 minute at room temperature to dry the membrane. Discarded the collection tube and insert the spin cartridge into a fresh eppendorf tube.

Added 100 μL RNase-Free Water to the center of the spin cartridge. Incubated at room temperature for 1 minute. Centrifuged the spin cartridge with the eppendorf tube for 2 minutes at >12,000×g at room temperature to elute the RNA.

The concentration of the eluted RNA was measured using a Nanodrop.

The extracted RNA was then DNase treated to remove any contaminating DNA using the Thermo Fisher TURBO-DNA Free kit as follows:

Prepared a 50 μl reaction containing less than 50 μg RNA. The same concentration of RNA was added to the reaction for each sample, calculated from the Nanodrop RNA concentration to standardize the amount of RNA across all the samples. Mixed up the reactions in Eppendorf tubes with the volume of RNA required, 5 μl Turbo DNase buffer, 2 μl Turbo DNase and then made up the volume to 50 μl with nuclease free water whilst keeping all the reagents on ice. A no RNA control sample was prepared to use as a blank when measuring the concentration of the DNase treated RNA samples using the nanodrop. Eppendorf tubes were incubated at 37° C. for 30 minutes. The reaction was then inactivated by the addition of 10 μl DNase inactivation reagent and mixed well. Eppendorf tubes were incubated at room temperature for 5 mins, inverted 2-3 times to mix reagents during incubation. Samples were the centrifuged at 10,000×g for 1.5 minutes. The supernatant was then transferred into a fresh Eppendorf tube which was also centrifuged, and the supernatant was transferred again into a fresh Eppendorf tube. The concentration of the DNase treated RNA samples was then measured using the nanodrop, using the no-RNA control sample as a blank.

DNase treated RNA samples were then checked for DNA contamination by PCR, using SalB primers, which would produce a DNA band of approximately 500 bp in size.

PCR reactions were prepared in 0.2 ml PCR tubes for each DNase treated RNA sample, a positive control sample of extracted DNA from S. salivarius K12 and a negative control sample containing nuclease free water only. 25 μl reactions were prepared by mixing 12.5 μl GoTaq G2 Hot start green Master mix, 1 μl SalB forward primer, 1 μl SalB reverse primer, 1 μl of either RNA or DNA sample and nuclease free water up to a volume of 25 μl.

PCR amplification consisted of initial denaturation 15 min at 94° C., followed by 30 cycles of. Denaturation—30 secs at 95° C.; Annealing—30 secs at 40° C.; Extension—30 secs at 73° C. After the 30 cycles, another 2 min at 92° C.

After PCR amplification a 0.5 cm thick 2% agarose/lx TAE gel containing 1×SYBR safe DNA gel stain and a 1.5 mm width comb was loaded with either 10 μl of each DNase treated RNA sample or 5 μl of the AccuRuler 1kb DNA RTU ladder into a well to determine the band size of any visualised bands.

No DNA bands detected in the DNase treated samples confirming that any DNA contamination was removed.

Next RNA samples were converted to cDNA using the superscript IV vilo master mix, following the manufactures instructions as follows:

Prepared 20 μl reactions in 0.2 ml PCR tubes containing up to 2.5 μg RNA, normalised the amount of RNA added, so that all the samples had the same concentration of RNA in the tubes. To each tube 4 μl of superscript IV vilo master mix was added and the volume was made up to 20 μl with nuclease free water. A duplicate set of reactions were prepared containing the same concentration of RNA for each sample but with the addition of 4 μl of superscript IV vilo No RT control and the volume was also made up to 20 μl with nuclease free water.

PCR tubes were then placed in an PCR machine to conduct the following incubations: 10 minutes at 25° C. (primer annealing); 10 minutes at 50° C. (reverse transcribe RNA); 5 minutes at 85° C. (inactivate enzyme).

The cDNA samples from K12 raw ingredient grown on the CABCa control plates and CABCa supplemented with the various saccharides were then analysed by qPCR for the levels of gene expression of the following genes: salA, salB, salQ and ureC.

10 μl qPCR reactions were prepared containing 2 μl of cDNA, 5 μl SYBR green master mix, 0.5 μl forward primer, 0.5 μl reverse primer and nuclease free water up to 10 μl. The dilution of cDNA in each qPCR was optimised with each primer set.

qPCR method consisted of the following cycles: Hold Stage: 2 mins at 50° C. followed by 10 mins at 95° C.; PCR Stage: 15 secs at 95° C. followed by 1 min at 60° C.; Melt Curve Stage: 15 secs at 95° C. followed by 1 min at 60° C. followed by 15 secs at 95° C.

To analyse the relative expression levels the 2^{-ΔΔCt} method was used to determine the relative fold gene expression level comparing the different sugars to a CABCa plate control. The reference gene used for this analysis was gyrA.

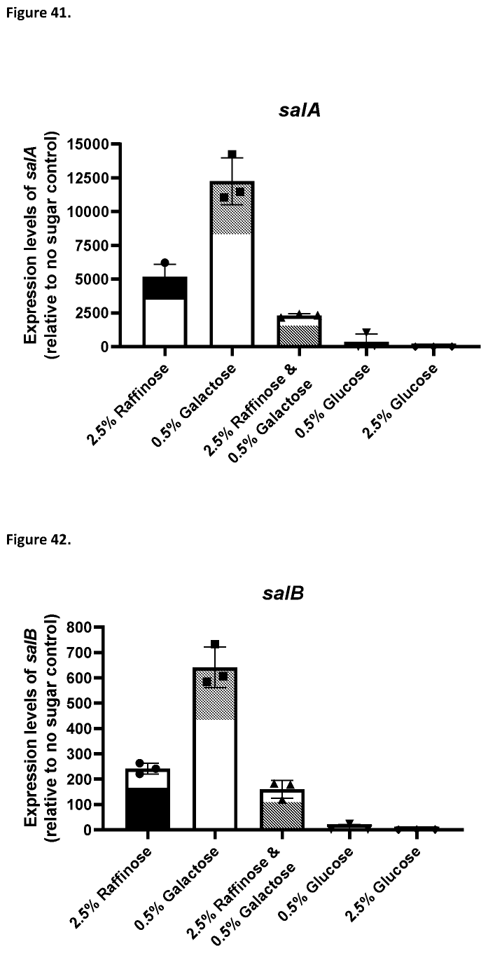

Results: Both salA and salB genes were upregulated in K12 were relative to a no sugar control when either 2.5% w/w raffinose, 0.5% w/w galactose or the combination of both was added (FIGS. 41 and 42). Interestingly 0.5% w/w galactose seemed to cause the greatest increase in gene expression (˜12000 fold) of salA and (641 fold) of salB, followed by 2.5% w/w raffinose (˜5000 and 241 fold) and then the combination (˜2400 and 160 fold). The upregulation of theses gene did not occur to the same extent when a control sugar glucose was added in either 0.5% w/w (˜50 and 23 fold) or 2.5% w/w (5.2 and 0.8 fold) concentrations.

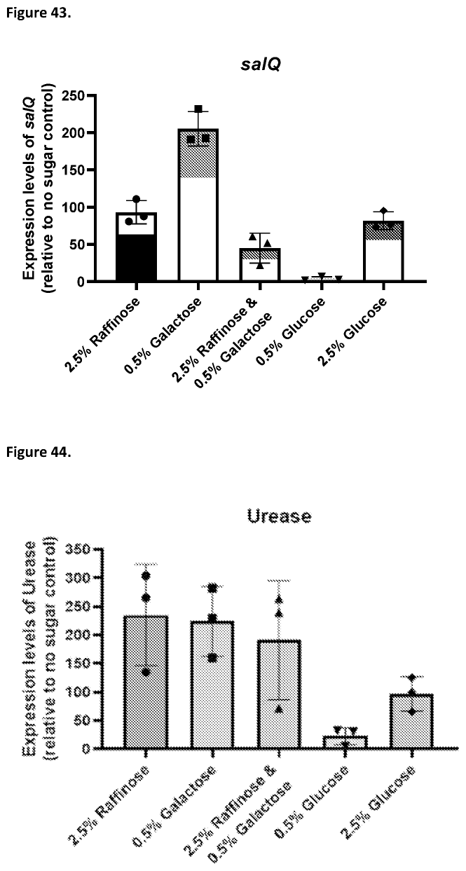

Additionally, salQ was upregulated relative to a no sugar control for when either 2.5% w/w raffinose (93 fold), 0.5% w/w galactose (205 fold) or the combination of both (45 fold) was added (FIG. 43). However, salQ also appears to be upregulated when 2.5% w/w glucose (81 fold) is added indicating that this may be a general sugar effect.

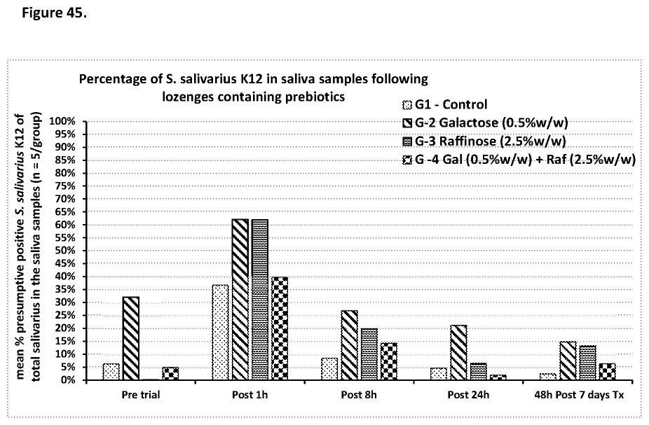

When either 2.5% w/w raffinose, 0.5% w/w galactose or the combination of both was added to the medium there was an upregulation in urease (ureC) expressed with increases of 234 fold, 224 fold and 191 fold respectively (FIG. 44). 0.5% w/w and 2.5% w/w glucose also gave moderate increases in urease expression of 22 fold and 96 fold respectively.

### Example 20: Change in the Level of K12 Colonisation in the Oral Cavity of Healthy Human Volunteers

Aim: To determine if the addition of galactose and/or raffinose to a commercial S. salivarius K12 lozenge formulation (Throat Guard Pro, Blis Technologies (NZ)) will change the colonisation level of K12 when consumed once a day for 7 days.

Method: A double-blind, randomized controlled colonization pilot study with no cross over was conducted in healthy human adults to evaluate the colonization efficacy of lozenges containing S. salivarius K12 (˜2.5 Billion cfu/lozenge) without galactose or raffinose (control G1, containing S. salivarius K12, isomalt, tableting aids and natural flavour Blis Technologies (NZ)) and lozenges additionally containing: raffinose 2.5% w/w (G2), galactose 0.5% w/w (G3), and a combination of galactose 0.5% w/w and raffinose 2.5% w/w (G4).

Lozenges G2-G4 were prepared by blending S. salivarius K12, isomalt, tableting aids and natural flavour, with: raffinose 2.5% w/w (G2); galactose 0.5% (G3); and galactose 0.5% w/w and raffinose 2.5% w/w (G4). Each blend was then subjected to the tableting machine to obtain lozenges. Each of the lozenges in the four groups was formulated to contain about 2.5 billion cfu/lozenge.

Participants were enrolled if they were healthy and practice good oral hygiene, 18-80 years of age, not on antibiotic therapy, immunocompromised or on history of autoimmune disease, people with allergy or sensitivity to dairy. Following the inclusion criteria, a total of 20 participants were recruited and divided into 4 groups.
Study Group: K12 Lozenges (2.5B cfu/lozenge)
G1: K12 lozenges (control) (n=5)
G2: K12 lozenges containing raffinose (2.5% w/w) (n=5)
G3: K12 lozenges containing galactose (0.5% w/w) (n=5)
G4: K12 lozenges containing raffinose (2.5% w/w) and galactose (0.5% w/w) (n=5)

Participants were asked to gargle with mouthwash (only on the first night), wait 1 h and then collect saliva sample (pre-trial sample). They were then asked to slowly dissolve one lozenge in the mouth. Participants were then asked to collect further saliva samples 1 hour, 8 hours and 24 hours after taking the lozenge. Participants were then asked to take 1 lozenge per night for 6 more nights, and collect a final saliva sample 48h after the last dose.

### Microbial Sampling and Analysis:

During and at the end of the trial, the tubes containing saliva samples for each time point and each participant were collected and stored in a freezer (−20° C.) until analysed.

Saliva samples were serially diluted (multiple repeats of 100 μL sample resuspended in 900 μL of PBS) to 10⁴ and spread plated on Mitis-Salivarius agar plates (a Streptococcus salivarius selective media) using a 50 μL inoculum per plate. The plates were incubated for 24 h at 37° C., 5% CO₂ in air. After incubation K12 or M18 colonies were differentiated by their inhibition activity against the specific indicator strains I1 (Micrococcus luteus T-18) and 13 Streptococcus constellatus T-29). Suspensions of the indicator strain I1 was made by the addition of 1 colony to 3 ml of THB, and the 13 suspension was made by the addition of 4 colonies to 3 ml of THB. The indicator strains were swabbed on to blood agar plates (sBaCa) covering the entire surface of the agar. Using a toothpick, the S. salivarius like colonies grown from the saliva samples on the mitis salivarius agar were spiked into the I1 then I3 pre-seeded indicator lawns and incubated for 24 h at 37° C., 5% CO₂ in air. Colonies with an inhibition zone for both I1 and 13 were identified as presumptive positive K12 as they indicate the activity of the salA and salB genes.

### Results

FIG. 45 shows that the average percentage of S. salivarius K12 (of total S. salivarius) in the saliva samples obtained from the groups of participants using the lozenges with supplemental saccharide galactose (G2), raffinose (G3) and raffinose and galactose (G4) was greater than the average percentage for the control group with S. salivarius K12 alone (Control G1). Thus, the presence of the supplemental saccharides enhanced the colonisation efficacy of S. salivarius K12 in the oral cavity.

In the supplemental saccharide groups, raffinose (G3) showed the maximum increase in colonisation followed by galactose (G2) and combination of raffinose and galactose (G4) compared to pre-trial levels for all sample points. The percentage of S. salivarius K12 in the total S. salivarius population remains higher for the raffinose group (G3). The level in the raffinose group was maintained higher then pre-trial even after the cessation of lozenge consumption, suggesting improved persistence of S. salivarius K12 due to the supplemental saccharides.

It is not the intention to limit the scope of the invention to the abovementioned examples only. As would be appreciated by a skilled person in the art, many variations are possible without departing from the scope of the invention as set out in the appended claims.

1. A method of improving the inhibitory profile of Streptococcus salivarius comprising formulating the S. salivarius in a composition comprising an effective amount of a supplemental saccharide,
wherein the Streptococcus salivarius is Streptococcus salivarius M18, Streptococcus salivarius K12, or a combination thereof, and
wherein the supplemental saccharide is galactose, or raffinose, or a combination thereof.

2. A method for upregulating one or more genes in Streptococcus salivarius, comprising formulating the S. salivarius in a composition comprising an effective amount of a supplemental saccharide,
wherein the Streptococcus salivarius is Streptococcus salivarius M18, Streptococcus salivarius K12, or a combination thereof, and
wherein the supplemental saccharide is galactose, or raffinose, or a combination thereof.

3. A method according to claim 2, wherein the upregulated gene(s) encodes for a lantibiotic peptide or bacteriocin.

4. A method according to claim 3, wherein the upregulated gene(s) encodes for a Class I or Class II lantibiotic peptide or bacteriocin.

5. A method according to claim 4, wherein the lantibiotic peptide is salA, salB, sal9 or a combination thereof.

6. A method according to claim 4 or 5, wherein the lantibiotic peptide is salA, salB, or a combination thereof.

7. A method according to claim 4, wherein the bacteriocin is salQ.

8. A method according to claim 2, wherein the upregulated gene(s) encodes for a subunit of a urease protein.

9. A method according to claim 8, wherein the upregulated gene is ureC.

10. A method according to any one of claims 2 to 9, wherein at least one of the upregulated gene(s) comprises or consists of a polynucleotide sequence with at least 70% sequence identity to any one of SEQ ID NOs 15-22, or wherein at least one of the upregulated gene(s) comprises or consists of a polynucleotide sequence that encodes a polypeptide with at least 70% sequence identity to any one of SEQ ID NOs 23-30.

11. The method of claim 10, wherein at least one of the upregulated gene(s) comprises or consists of a polynucleotide sequence with at least 75% identity to any one of SEQ ID NOs 15-22, preferably at least 80%, 85%, 90%, 95%, or 99% identity to any one of SEQ ID NOs 15-22.

12. The method of claim 10 or 11, wherein at least one of the upregulated gene(s) comprises or consists of a polynucleotide sequence that encodes a polypeptide with at least 75% identity to any one of SEQ ID NOs 23-30, preferably at least 80%, 85%, 90%, 95%, or 99% identity to any one of SEQ ID NOs 23-30.

13. The method of any one of claims 10 to 12, wherein:
a. at least one of the upregulated gene(s) is a salA gene or variant thereof comprising or consisting of a polynucleotide sequence with least 70% identity to SEQ ID NO 15 or 19, or encoding a polypeptide with at least 70% identity to SEQ ID NO 23 or 27;
b. at least one of the upregulated gene(s) is a salB gene or variant thereof comprising or consisting of a polynucleotide sequence with at least 70% identity to SEQ ID NO 16, or encoding a polypeptide with at least 70% identity to SEQ ID NO 24;
c. at least one of the upregulated gene(s) is a salQ gene or variant thereof comprising or consisting of a polynucleotide sequence with at least 70% identity to SEQ ID NO 17 or 21, or encoding a polypeptide with at least 70% identity to SEQ ID NO 25 or 29;
d. at least one of the upregulated gene(s) is a sal9 gene or variant thereof comprising or consisting of a polynucleotide sequence with at least 70% identity to SEQ ID NO 20, or encoding a polypeptide with at least 70% identity to SEQ ID NO 28; and/or
e. at least one of the upregulated gene(s) is a ureC gene or variant thereof comprising or consisting of a polynucleotide sequence with at least 70% identity to SEQ ID NO 18 or 22, or encoding a polypeptide with at least 70% identity to SEQ ID NO 26 or 30.

14. A method for increasing production of one or more of a lantiobiotic peptide, bacteriocin or urease by Streptococcus salivarius, comprising formulating the S. salivarius in a composition comprising an effective amount of a supplemental saccharide,
wherein the Streptococcus salivarius is Streptococcus salivarius M18, Streptococcus salivarius K12, or a combination thereof, and
wherein the supplemental saccharide is galactose, or raffinose, or a combination thereof.

15. A method according to claim 14, wherein the lantibiotic peptide or bacteriocin is a Class I or Class II lantibiotic peptide or bacteriocin.

16. A method according to claim 15, wherein the lantibiotic peptide is salA, salB, sal9 or a combination thereof.

17. A method according to claim 15 or 16, wherein the lantibiotic peptide is salA, salB, or a combination thereof.

18. A method according to claim 11, wherein the bacteriocin is salQ.

19. A method according to any one of claims 14 to 18, which increases production of a polypeptide with at least 70% sequence identity to any one of SEQ ID NOs 23-30.

20. The method of claim 19, wherein at the polypeptide has at least 75% identity to any one of SEQ ID NOs 23-30, preferably at least 80%, 85%, 90%, 95%, or 99% identity to any one of SEQ ID NOs 23-30.

21. The method of any one of claims 1-20, wherein the method increases the inhibitory profile of S. salivarius against skin, dental, oral, mucosal and/or ENTR microorganisms.

22. The method of claim 21, wherein the skin, oral, dental, mucosal, and/or ENTR microorganism is selected from S. aureus spp., S. intermedius spp., S. saprophyticus spp., M. catarrhalis spp., H. influenzae spp., S. pyogenes spp., P. aeruginosa spp., S. mutans spp., S. pneumoniae spp., C. acnes spp., C. albicans spp. S. sobrinus spp., Corynebacterim spp., F. nucleatum spp., A. actinomycetemcomitans spp., P. gingivalis spp., Tannerella forsythia spp., Treponema denticola spp., P. intermedia spp., Prevotella spp., A. viscosus spp., S. equismillis spp., S. dygalactiae spp., S. sanguis spp., S. cohnii spp., B. intermedius spp., A. parvulum spp., E. saburreum spp., E. sulci spp., P. micra spp., S. moorei spp., S. agalactiae spp., C. minutissimus spp., P. propionicus spp., S. agalactiae spp., S. dysgalactiae spp., S. simulans spp., S. xylosus spp., Tinea pedis infection causing fungi, S. salivarius spp. other than K12 or M18, L. lactis spp., S. epidermidis spp., S. constellatus spp., K. pneumoniae spp., A. baumanii spp. or any combination of any two or more thereof.

23. The method of claim 22, wherein the microorganism is selected from S. aureus A222, S. aureus 20, S. aureus 14, S. aureus 19, S. aureus A504, S. saprophyticus ATCC 15305, M. catarrhalis TW1, M. catarrhalis TW2, H. influenzae TW5, S. pyogenes M76, S. pyogenes 71-698, S. pyogenes FF22, S. pyogenes 71-679, S. pyogenes W-1, S. pyogenes M17, S. pyogenes M57, S. pyogenes EMM92, S. pyogenes M66, S. pyogenes M74, P. aeruginosa 12, S. mutans OMZ175, S. pneumoniae D39, L. lactis T-21, S. epidermidis 11, S. constellatus T-29, S. salivarius 6, S. salivarius 193, S. salivarius 20P3, or any combination of any two or more thereof.

24. The method of any one of claims 1 to 23, wherein the composition comprises at least about 0.1% by weight of each S. salivarius.

25. The method of any one of claims 1 to 24, wherein the composition comprises from about 0.1 to about 20% by weight of each S. salivarius.

26. The method of any one of claims 1 to 25, wherein the composition comprises at least about 1×10³ cfu/g of each S. salivarius.

27. The method of any one of claims 1 to 26, wherein the composition comprises from about 1×10³ to about 1×10¹³ cfu/g of each S. salivarius.

28. The method of any one of claims 1 to 27, wherein the composition comprises less than about 20% by weight of each supplemental saccharide.

29. The method of any one of claims 1 to 28, wherein the composition comprises from about 0.1 to about 20% by weight of each supplemental saccharide.

30. The method of any one of claims 1 to 29, wherein the composition is formulated for oral, dental, nasal, mucosal, topical, or pulmonary administration.

31. The method of any one of claims 1 to 30, wherein the composition is formulated in a slow-release composition.

32. The method of any one of claims 1 to 31, wherein the composition is formulated into a powder, lozenge, nasal spray, nasal gel, nasal drop, oral drop, oral gel, oral spray, inhalable, topical composition, chewable, melt, film, gummy, toothpaste, tooth-gel, varnish, mousse, mouthwash, food product (e.g. yoghurt), cream, gel spray, deodorant, serum, lotion, balm, moisturiser, pessary, or suppository.

33. A method of inhibiting a skin, dental, oral, mucosal and/or ENTR microorganism, the method comprising contacting the microorganism with a composition comprising Streptococcus salivarius and an effective amount of a supplemental saccharide,
wherein the Streptococcus salivarius is Streptococcus salivarius M18, Streptococcus salivarius K12, or a combination thereof, and
wherein the supplemental saccharide is galactose or raffinose or a combination thereof.

34. The method of claim 33, wherein the microorganism is a Streptococcus or Staphylococcus bacteria selected from S. aureus spp., S. saprophyticus spp., S. mutans spp., S. pyogenes spp., S. pneumoniae spp.; and the S. salivarius strain is K12.

35. The method of claim 33 or 34, wherein the Streptococcus or Staphylococcus bacteria is selected from S. aureus A222, S. saprophyticus ATCC 15305, S. mutans OMZ175, S. constellatus T-29, S. pyogenes 71-698, and S. pneumoniae D39; and the S. salivarius strain is K12.

36. The method of any one of claims 33 to 35, wherein the supplemental saccharide is raffinose and is present in the composition in an amount of 0.5 to 15%, or 1 to 12%, or 1.5 to 10%, or 2 to 7%, or 2.5 to 5% by weight.

37. The method of any one of claims 33 to 36, wherein the supplemental saccharide is galactose and is present in the composition in an amount of 0.5 to 15%, or 1 to 12%, or 1.5 to 10%, or 2 to 7%, or 2.5 to 5% by weight.

38. The method of any one of claims 33 to 37, wherein the bacteria are selected from S. pyogenes spp., and S. pneumoniae spp.; and the S. salivarius strain is M18.

39. The method of any one of claims 33 to 38, wherein the bacteria is selected from S. pyogenes 71-698, and S. pneumoniae D39; and the S. salivarius strain is M18.

40. The method of any one of claims 33 to 37, wherein the bacteria is selected from S. constellatus, S. mutans, and S. saprophyticus; and the S. salivarius strain is M18.

41. The method of any one of claims 33 to 37, wherein the bacteria is selected from S. constellatus T29, S. mutans OMZ175, and S. saprophyticus ATCC 15305; and the S. salivarius strain is M18.

42. The method of any one of claims 33 to 41, wherein the supplemental saccharide is raffinose and is present in the composition in an amount of 0.25 to 10%, or 0.5 to 8%, or 0.75 to 7%, or 1 to 6%, or 1.25 to 5% by weight.

43. The method of any one of claims 33 to 41, wherein the supplemental saccharide is galactose and is present in the composition in an amount of 0.25 to 10%, or 0.5 to 8%, or 0.75 to 7%, or 1 to 6%, or 1.25 to 5% by weight.

44. A composition comprising Streptococcus salivarius and an effective amount of a supplemental saccharide for use in improving the inhibitory profile of the Streptococcus salivarius,
wherein the Streptococcus salivarius is Streptococcus salivarius M18, Streptococcus salivarius K12, or a combination thereof, and
wherein the supplemental saccharide is galactose or raffinose or a combination thereof.

45. A composition according to claim 44, comprising one or more of: galactose in an amount of 0.1 to 1%, or 0.2 to 0.8, or 0.25 to 0.75, or at 0.5% by weight, and raffinose in an amount of 0.5 to 5%, or 1 to 4, or 2 to 3, or 2.5% by weight.

46. A composition comprising Streptococcus salivarius K12, and raffinose in an amount of 2 to 3% by weight.

47. A composition comprising Streptococcus salivarius K12, and galactose in an amount of 0.25 to 0.75% by weight.

48. A composition comprising Streptococcus salivarius M18, and raffinose in an amount of 2 to 3% by weight.

49. A composition comprising Streptococcus salivarius M18, and galactose in an amount of 0.25 to 0.75% by weight.

50. A composition comprising Streptococcus salivarius K12, Streptococcus salivarius M18, and raffinose in an amount of 2 to 3% by weight.

51. A composition comprising Streptococcus salivarius K12, Streptococcus salivarius M18, and galactose in an amount of 0.25 to 0.75% by weight.

52. A composition comprising Streptococcus salivarius K12, Streptococcus salivarius M18, raffinose in an amount of 2 to 3% by weight, and galactose in an amount of 0.25 to 0.75% by weight.

53. A composition comprising Streptococcus salivarius K12, raffinose in an amount of 1.2 to 2.2% by weight, and galactose in an amount of 0.7 to 1.7% by weight.

54. A composition comprising Streptococcus salivarius M18, raffinose in an amount of 1.2 to 2.2% by weight, and galactose in an amount of 0.7 to 1.7% by weight.

55. A composition comprising Streptococcus salivarius K12, Streptococcus salivarius M18, raffinose in an amount of 1.2 to 2.2% by weight, and galactose in an amount of 0.7 to 1.7% by weight.

56. The composition according to any one of claims 44 to 55, further comprising one or more of a carrier; a tableting aid, including a binder or a lubricant; and a flavouring agent.

57. A therapeutic formulation comprising the composition of any one of claims 44 to 56.

58. The therapeutic formulation of claim 57, wherein the therapeutic formulation is formulated for oral, dental, nasal, mucosal, topical, or pulmonary administration.

59. The therapeutic formulation of claim 57 or 58, wherein the therapeutic formulation is a slow-release composition.

60. The therapeutic formulation of any one of claims 57 to 59, wherein the therapeutic formulation is a powder, lozenge, nasal spray, nasal gel, nasal drop, oral drop, oral gel, oral spray, inhalable, topical composition, chewable, melt, film, gummy, toothpaste, tooth-gel, varnish, mousse, mouthwash, food product (e.g. yoghurt), cream, gel, spray, deodorant, serum, lotion, balm, moisturiser, pessary, or suppository.

61. The therapeutic formulation of claim 60, which is a powder.

62. The therapeutic formulation of claim 60, which is a lozenge.

63. A method of treating or preventing a disease or disorder comprising administering to subject in need thereof a composition of any one of claims 44 to 56, or a therapeutic formulation of any one of claims 57 to 62.

64. The method of claim 63, wherein the disease or disorder is caused by an oral, dental, mucosal, skin, or ENTR pathogen.

65. The method of claim 63 or 64, wherein the disease or disorder is caused by a pathogenic Streptococcus or Staphylococcus bacteria.

66. The method of any one of claims 63 to 65, wherein the pathogenic Streptococcus or Staphylococcus bacteria is selected from S. aureus spp., S. saprophyticus spp., S. mutans spp., S. pyogenes spp., and S. pneumoniae spp.

67. The method of any one of claims 63 to 66, wherein the disease or disorder is selected from otitis media, sore throat, tooth decay, acute pharyngitis, tonsillitis, pneumonia, COPD, periodontal disease, gingivitis, halitosis, dental caries, sepsis, meningitis, candidiasis (oral thrush), vaginitis, body odour, acne, actinomycosis, psoriasis, erythrasma, cellulitis, impetigo, atopic dermatitis, bacteraemia, tineas including athlete's foot, soft tissue infections, erythema, nosocomial, erythema, SARS-CoV, influenza A, influenza B, and RSV or any combination of any two or more thereof.

68. A method of inhibiting a microorganism sensitive to Blis-producing S. salivarius, the method comprising administering to subject in need thereof a composition of any one of claims 33 to 37, or a therapeutic formulation of any one of claims 57 to 62.

69. The method of claim 68 wherein the microorganism sensitive to Blis-producing S. salivarius is selected from S. salivarius spp., S. epidermidis spp., S. constellatus spp., and L. lactis spp.

70. The method of claim 68 wherein the microorganism sensitive to Blis-producing S. salivarius is selected from S. pyogenes spp., F. nucleatum spp., and P. gingivalis spp.

71. The method of any one of claims 63 to 70, wherein the subject is a human.

72. Use of Streptococcus salivarius and a supplemental saccharide in the manufacture of a medicament for:
(a) the treatment or prevention of a disease or disorder, or
(b) the inhibition of a microorganism sensitive to Blis-producing S. salivarius,
wherein the Streptococcus salivarius is Streptococcus salivarius M18, Streptococcus salivarius K12, or a combination thereof, and
wherein the supplemental saccharide is galactose or raffinose or a combination thereof.

73. A composition comprising Streptococcus salivarius and an effective amount of a supplemental saccharide for use in:
(a) the treatment or prevention of a disease or disorder, or
(b) the inhibition of a microorganism sensitive to Blis-producing S. salivarius,
wherein the Streptococcus salivarius is Streptococcus salivarius M18, Streptococcus salivarius K12, or a combination thereof, and
wherein the supplemental saccharide is galactose or raffinose or a combination thereof.

74. The composition of claim 73, wherein the composition is a powder, lozenge, nasal spray, nasal gel, nasal drop, oral drop, oral gel, oral spray, inhalable, topical composition, chewable, melt, film, gummy, toothpaste, tooth-gel, varnish, mousse, mouthwash, food product (e.g. yoghurt), cream, gel, spray, deodorant, serum, lotion, balm, moisturiser, pessary, or suppository.

75. The composition of claim 74, which is a powder.

76. The composition of claim 74, which is a lozenge.

77. A composition according to any one of claims 44 to 56 or 73 to 75, wherein the composition is a cosmetic.

78. A composition according to any one of claims 44 to 56 or 73 to 76, wherein the composition is a dietary supplement.

79. A composition according to any one of claims 44 to 56 or 73 to 76, wherein the composition is a natural health product.

80. A composition according to any one of claims 44 to 56 or 73 to 76, wherein the composition is a complementary medicine.

81. A method of manufacturing a composition comprising Streptococcus salivarius and an effective amount of a supplemental saccharide, the method comprising:
(a) combining Streptococcus salivarius with supplemental saccharide, and
(b) mixing to produce a homogeneous blend:
wherein the Streptococcus salivarius is Streptococcus salivarius M18, Streptococcus salivarius K12, or a combination thereof, and
wherein the supplemental saccharide is galactose or raffinose or a combination thereof.

82. The method of claim 81, wherein the composition is a lozenge, and the method further comprises a step of lozenging the homogeneous blend to produce the lozenge.

83. The method of claim 81 or 82, wherein the inhibitory profile of the S. salivarius in the composition is improved relative to a composition lacking the supplemental saccharide.

84. The method of claim any one of claims 81 to 83, wherein the composition is for use in:
(a) the treatment or prevention of a disease or disorder, or
(b) the inhibition of a microorganism sensitive to Blis-producing S. salivarius.

85. Use of a composition manufactured by the method of any one of claims 81 to 84 for the treatment or prevention of a disease or disorder, or for the inhibition of a microorganism sensitive to Blis-producing S. salivarius.


SEQUENCE LISTING (30 sequences)

SEQ ID NO: 1 - DNA, 20 bp, synthetic construct
  source 1..20: mol_type = other DNA
tgagtcaagccatgcgtaag

SEQ ID NO: 2 - DNA, 20 bp, synthetic construct
  source 1..20: mol_type = other DNA
caggggtcgtttctggatta

SEQ ID NO: 3 - DNA, 21 bp, synthetic construct
  source 1..21: mol_type = other DNA
ccgttacatgttgggttgacg

SEQ ID NO: 4 - DNA, 20 bp, synthetic construct
  source 1..20: mol_type = other DNA
atcacgaagcatttccaagg

SEQ ID NO: 5 - DNA, 21 bp, synthetic construct
  source 1..21: mol_type = other DNA
tgattgccatgaaaaactcaa

SEQ ID NO: 6 - DNA, 21 bp, synthetic construct
  source 1..21: mol_type = other DNA
ttgcaaaccaacctgtacctc

SEQ ID NO: 7 - DNA, 20 bp, synthetic construct
  source 1..20: mol_type = other DNA
acactcgatctcgaggcact

SEQ ID NO: 8 - DNA, 20 bp, synthetic construct
  source 1..20: mol_type = other DNA
gaatgtggcataaccaacta

SEQ ID NO: 9 - DNA, 22 bp, synthetic construct
  source 1..22: mol_type = other DNA
ctcgttgagattgaagcaatga

SEQ ID NO: 10 - DNA, 20 bp, synthetic construct
  source 1..20: mol_type = other DNA
ttcgtgtgaaatggtttgga

SEQ ID NO: 11 - DNA, 20 bp, synthetic construct
  source 1..20: mol_type = other DNA
tttccaaggtcgtaccgttc

SEQ ID NO: 12 - DNA, 20 bp, synthetic construct
  source 1..20: mol_type = other DNA
tggatccaagtggtgacaaa

SEQ ID NO: 13 - DNA, 27 bp, synthetic construct
  source 1..27: mol_type = other DNA
gtgaattctcttcaagaattgactctt

SEQ ID NO: 14 - DNA, 22 bp, synthetic construct
  source 1..22: mol_type = other DNA
aaaatattcataccgctcttcc

SEQ ID NO: 15 - DNA, 156 bp, Streptococcus salivarius
  source 1..156: mol_type = genomic DNA
atgattgccatgaaaaactcaaaagatattttgaacaatgctatcgaagaagtttctgaaaaagaacttatggaagtagctggtggtaaaagaggtacaggttggtttgcaactattactgatgactgtccaaactcagtattcgtttgttgttaa

SEQ ID NO: 16 - DNA, 186 bp, Streptococcus salivarius
  source 1..186: mol_type = genomic DNA
gtgaggtatttccaaatggctaaacaacaaatgaatctcgttgagattgaagcaatgaattctcttcaagaattgactcttgaagaacttgataacgttcttggtgctggtggtggagtaatccaaaccatttcacacgaatgtcgcatgaactcatggcagttcttgtttacttgttgctcttaa

SEQ ID NO: 17 - DNA, 180 bp, Streptococcus salivarius
  source 1..180: mol_type = genomic DNA
atgacaacacaaacaatgaacaattttgaaacactcgatctcgaggcacttgcaaacgttgaaggtggcggatgggttaagtgttatgccggcacaatcggatctgctctagtaggatcagctggaggcccagtaggttactggggaggagctttagttggttatgccacattctgctaa

SEQ ID NO: 18 - DNA, 1719 bp, Streptococcus salivarius
  source 1..1719: mol_type = genomic DNA
atgagttttaaaatggatcgtgaagagtatgctcaacactatggaccaactgtaggggatagcatacgtcttggagataccaatctttttgcagccattgaaaaagactttactgtttatggacaggaatctaagttcggtggcggtaaagttttgcgtgatggtatgggtgttagtgctacggaaacacgtgacaatccatcagttgttgataccattattacaggtgcaaccattattgactatacaggtattattaaagcagatatcggtattcgtgatggtaagattgttgctattggtcgcggtggtaacccagatacaatggacaatgtggactttgttgtgggtgctagtacagaagccattgctgcagaaggtttgattgtgactgctggtggtattgacctccacgtacattatatttctgctgaccttcctgaatttggtttggataatggaattacgaccctctttggtggtggtactggtcccgctgacggtagtaatgcaacaacatgtacaccaggtaaattccatattactcgtatgttgcaagctgttgatgatatgcctgctaactttggtttccttgccaaaggtgttggttctgagactgaagtggttgaagagcaaattaaagctggtgcagcaggaattaagacacacgaggactggggtgcgacttacgcaggaattgataattcccttaaagttgcggataaatacgatgtttcttttgcggtccacaccgactctttgaatgagggtggatttatggaaaatactttggaatctttccaaggtcgtaccgttcataccttccataccgaaggttctggtggtgggcacgctccagatattatggtctttgcaggtaaggaaaatattttgccatcatcaactaacccaactaacccatatactacaaatgctattggtgagttgttagatatggttatggtttgtcaccacttggatccaaaaatcccagaagacgtatcttttgccgaatcacgtgttcgtaaacaaactgtggctgcagaagatgttcttcacgatatgggagcccttagtatcatgacttcagatgccatggcaatgggacgtgtcggtgaagtagcaatgcgttgttggcaactagctgataagatgaaggctcagcgtggtccacttgaaggggattcagagtttaacgataataaccgtattaaacgttacgtggctaaatatacaattaaccctgccatcaccaatggtattgcagactatatcggttctgtagaagttggtaaatttgcagatttggttatctgggaaccagcacaatttggtgcaaaacctaagttggtgcttaagggtggtatgttaacttatggtgttatgggtgacgctggttcaagtcttccaacacctcaaccacgtatcatgcgtaaattatatggtgcttatggtcaagcggttcatgaaacaaatcttacatttgtctctcaatatgcttatgatcacggtatcaaagaagaaattggtttgaataagattgttcttcctgttaagaatacacgtaacttgactaagcgtgatatgaagcttaatgactacgctccaaaaacaatccgtattgatccacagacctttgatgtctttattgatgatgagttggttacttgtgagccaatccatacgacatcattgtctcaacgttatttcttgttctaa

SEQ ID NO: 19 - DNA, 156 bp, Streptococcus salivarius; identical to SEQ ID NO: 15
  source 1..156: mol_type = genomic DNA

SEQ ID NO: 20 - DNA, 171 bp, Streptococcus salivarius
  source 1..171: mol_type = genomic DNA
atgaaatcaacaaataatcaaagtatcgcagaaattgcagcagtaaactcactacaagaagtaagtatggaggaactagaccaaattattggtgctggaaacggagtggttcttactcttactcatgaatgtaacctagcaacttggacaaaaaaactaaaatgttgctaa

SEQ ID NO: 21 - DNA, 237 bp, Streptococcus salivarius
  source 1..237: mol_type = genomic DNA
atgacaacacaaatcattaacaattttaattcacttaattccgaagatctttctattatcgaaggtgggggtgtgattggttgtgtagctgggactgctggatctgctgggcttggatttttgactggaacatcagttggaactgttacatttccaattgttgggactgtctcaggtggagcatttggggcatggtcaggagctggccttggaatggctacattttgcggagtttag

SEQ ID NO: 22 - DNA, 1719 bp, Streptococcus salivarius
  source 1..1719: mol_type = genomic DNA
atgagttttaaaatggatcgtgaagagtatgctcaacactacggaccaactgtaggtgatagcgtacgtcttggagataccaatctttttgcagccattgaaaaagactttactgtttatggacaggaatctaagtttggtggcggtaaagttttgcgtgatggtatgggtgttagtgctacggaaacacgtgacaatccatcagttgttgacaccattattacaggtgcaaccatcattgactatacaggtattattaaagcagatatcggtattcgtgatggtaagattgttgctatcggtcgcggtggtaacccagatacaatggacaatgtggactttgttgtgggtgctagtacagaagccattgccgctgaaggtttgattgtgactgctggtggtattgaccttcacgtgcactatatttctgccgaccttcctgaatttggtttggataacgggattactaccctctttggtggtggtactggtcctgctgatggaagtaatgcgacaacttgtacaccaggtaaattccatattactcgtatgttgcaagctgttgatgatatgcctgctaactttggtttccttgccaaaggtgttggttctgagactgaagtggttgaagagcaaattaaggccggtgcagcaggaattaaaacacacgaggactggggtgcgacttacgcaggtattgataattcccttaaagttgcggataaatacgatgtttcctttgcggtccacactgactctttgaatgagggtggatttatggaaaatactttggaatcctttcaaggtcgtaccgttcataccttccacaccgaaggttcaggtggtggacatgctccagatatcatggtctttgctggtaaggaaaatattttgccatcatcaactaacccaactaacccatacactacaaatgctattggtgagttgttagatatggttatggtttgccaccacttggatccaaaaattccagaagacgtctcttttgctgaatcacgtgtacgtaaacaaactgtagctgcagaagacgttcttcacgatatgggtgcccttagtatcatgacttcagatgccatggcaatgggacgtgtcggtgaagtagccatgcgttgttggcaactagctgataagatgaaggctcagcgtggtccgcttgaaggggattcagagtttaacgataataaccgtatcaaacgttacgtggctaaatatacaattaaccctgctatcactaacggtattgcagactatatcggatctgtagaagttggtaaatttgcagatttggttatctgggaaccagcacaatttggtgcaaaacctaagttggtgcttaagggtggtatgttaacttatggtgttatgggtgacgctggttcaagtcttccaacacctcaaccacgtatcatgcgtaaattatatggtgcttacggtcaagcggttcatgaaacaaatcttacatttgtctctcaatacgcttatgatcacggtatcaaagaagaaattggtttgaataagattgttcttcctgttaagaatacgcgtaacttgactaagcgtgatatgaagcttaatgactacgctccaaaaacaatccgtatcgatccacagacctttgatgtctttattgatgatgagttggttacttgtgagccaatccatacgacatcattgtctcaacgttatttcttgttctaa

SEQ ID NO: 23 - protein, 51 aa, Streptococcus salivarius
MIAMKNSKDILNNAIEEVSEKELMEVAGGKRGTGWFATITDDCPNSVFVCC

SEQ ID NO: 24 - protein, 61 aa, Streptococcus salivarius
MRYFQMAKQQMNLVEIEAMNSLQELTLEELDNVLGAGGGVIQTISHECRMNSWQFLFTCCS

SEQ ID NO: 25 - protein, 59 aa, Streptococcus salivarius
MTTQTMNNFETLDLEALANVEGGGWVKCYAGTIGSALVGSAGGPVGYWGGALVGYATFC

SEQ ID NO: 26 - protein, 572 aa, Streptococcus salivarius
MSFKMDREEYAQHYGPTVGDSIRLGDTNLFAAIEKDFTVYGQESKFGGGKVLRDGMGVSATETRDNPSVVDTIITGATIIDYTGIIKADIGIRDGKIVAIGRGGNPDTMDNVDFVVGASTEAIAAEGLIVTAGGIDLHVHYISADLPEFGLDNGITTLFGGGTGPADGSNATTCTPGKFHITRMLQAVDDMPANFGFLAKGVGSETEVVEEQIKAGAAGIKTHEDWGATYAGIDNSLKVADKYDVSFAVHTDSLNEGGFMENTLESFQGRTVHTFHTEGSGGGHAPDIMVFAGKENILPSSTNPTNPYTTNAIGELLDMVMVCHHLDPKIPEDVSFAESRVRKQTVAAEDVLHDMGALSIMTSDAMAMGRVGEVAMRCWQLADKMKAQRGPLEGDSEFNDNNRIKRYVAKYTINPAITNGIADYIGSVEVGKFADLVIWEPAQFGAKPKLVLKGGMLTYGVMGDAGSSLPTPQPRIMRKLYGAYGQAVHETNLTFVSQYAYDHGIKEEIGLNKIVLPVKNTRNLTKRDMKLNDYAPKTIRIDPQTFDVFIDDELVTCEPIHTTSLSQRYFLF

SEQ ID NO: 27 - protein, 51 aa, Streptococcus salivarius; identical to SEQ ID NO: 23

SEQ ID NO: 28 - protein, 56 aa, Streptococcus salivarius
MKSTNNQSIAEIAAVNSLQEVSMEELDQIIGAGNGVVLTLTHECNLATWTKKLKCC

SEQ ID NO: 29 - protein, 78 aa, Streptococcus salivarius
MTTQIINNFNSLNSEDLSIIEGGGVIGCVAGTAGSAGLGFLTGTSVGTVTFPIVGTVSGGAFGAWSGAGLGMATFCGV

SEQ ID NO: 30 - protein, 572 aa, Streptococcus salivarius
MSFKMDREEYAQHYGPTVGDSVRLGDTNLFAAIEKDFTVYGQESKFGGGKVLRDGMGVSATETRDNPSVVDTIITGATIIDYTGIIKADIGIRDGKIVAIGRGGNPDTMDNVDFVVGASTEAIAAEGLIVTAGGIDLHVHYISADLPEFGLDNGITTLFGGGTGPADGSNATTCTPGKFHITRMLQAVDDMPANFGFLAKGVGSETEVVEEQIKAGAAGIKTHEDWGATYAGIDNSLKVADKYDVSFAVHTDSLNEGGFMENTLESFQGRTVHTFHTEGSGGGHAPDIMVFAGKENILPSSTNPTNPYTTNAIGELLDMVMVCHHLDPKIPEDVSFAESRVRKQTVAAEDVLHDMGALSIMTSDAMAMGRVGEVAMRCWQLADKMKAQRGPLEGDSEFNDNNRIKRYVAKYTINPAITNGIADYIGSVEVGKFADLVIWEPAQFGAKPKLVLKGGMLTYGVMGDAGSSLPTPQPRIMRKLYGAYGQAVHETNLTFVSQYAYDHGIKEEIGLNKIVLPVKNTRNLTKRDMKLNDYAPKTIRIDPQTFDVFIDDELVTCEPIHTTSLSQRYFLF